(19) United States
(12) Patent Application Publication
(10) Pub. No.: US 2024/0294484 A1
(43) Pub. Date: Sep. 5, 2024

(54) HERBICIDAL ARYLDIHYDROFURANE CARBOXYLATES

(71) Applicant: BASF SE, Ludwigshafen (DE)

(72) Inventors: Marc Heinrich, Ludwigshafen (DE); Gunther Zimmermann, Ludwigshafen (DE); Markus Kordes, Ludwigshafen (DE); Tobias Seiser, Ludwigshafen (DE); Trevor William Newton, Limburgerhof (DE); Gerd Kraemer, Limburgerhof (DE)

(21) Appl. No.: 18/571,819

(22) PCT Filed: Jun. 14, 2022
(86) PCT No.: PCT/EP2022/066067

§ 371 Date: Dec. 19, 2023

(30) Foreign Application Priority Data
Jun. 23, 2021 (EP) 21181068.4

(51) Int. Cl. C07D 307/30; A01N 43/08; A01P 13/00

(52) U.S. Cl. CPC C07D 307/30; A01N 43/08; A01P 13/00

(57) ABSTRACT

The invention relates to compounds of formula (I), and their use as herbicides. In said formula, R¹ to R¹⁰ represent groups such as hydrogen, halo-gen or linear or cyclic organic groups such as alkyl, alkenyl, alkynyl, cycloalkyl, or alkoxy. The invention further refers to a composition comprising such compound and to the use thereof for controlling unwanted vegetation.

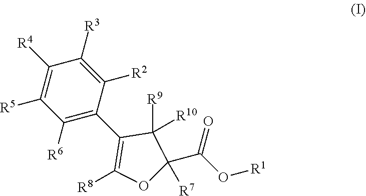

The present invention relates to aryldihydrofurane carboxylates and compositions comprising the same. The invention also relates to the use of the aryldihydrofurane carboxylates or the corresponding compositions for controlling unwanted vegetation. Furthermore, the invention relates to methods of applying the aryldihydrofurane carboxylates or the corresponding compositions. For the purpose of controlling unwanted vegetation, especially in crops, there is an ongoing need for new herbicides that have high activity and selectivity together with a substantial lack of toxicity for humans and animals.

WO12130798, WO1404882, WO14048882, WO18228985, WO18228986, WO19034602, WO19145245, WO20114932, WO20114934 and WO20182723 describe 3-phenylisoxazoline-5-carboxamides and their use as herbicides.

The synthesis of 2,3-dihydro-4-phenyl-2-furancarboxylates has been described in J. Org. Chem. 1973, 38, 2319-28 by using copper(I) carbenoid intermediates.

The compounds of the prior art often suffer from insufficient herbicidal activity, in particular at low application rates, and/or unsatisfactory selectivity resulting in a low compatibility with crop plants.

Accordingly, it is an object of the present invention to provide compounds having a strong herbicidal activity, in particular even at low application rates, a sufficiently low toxicity for humans and animals and/or a high compatibility with crop plants. The aryldihydrofurane carboxylates should also show a broad activity spectrum against a large number of different unwanted plants.

These and further objectives are achieved by the compounds of formula (I) defined below including their agriculturally acceptable salts and thioesters.

Accordingly, the present invention provides compounds of formula (I)

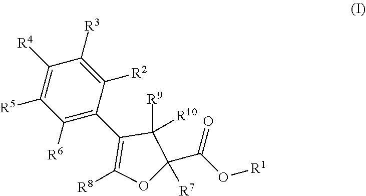

wherein the substituents have the following meanings:
R¹ hydrogen or (C₁-C₆)-alkyl, (C₃-C₆)-cycloalkyl, (C₃-C₆)-cycloalkyl-(C₁-C₃)-alkyl, (C₂-C₄)-alkenyl, (C₂-C₄)-alkynyl, each of which is substituted by m radicals selected from the group consisting of fluorine, chlorine, bromine, cyano, Z, CO₂R^{a}, CONR^{b}R^{h}, (C₁-C₂)-alkoxy, (C₁-C₂)-haloalkoxy, (C₁-C₃)-alkylthio, (C₁-C₃)-alkylsulfinyl, (C₁-C₃)-alkylsulfonyl, phenylthio, phenylsulfinyl, and phenylsulfonyl;
R² hydrogen, halogen, (C₁-C₃)-alkyl, (C₁-C₃)-haloalkyl, (C₁-C₃)-alkoxy, (C₁-C₃)-haloalkoxy;
R³ hydrogen, halogen, nitro, hydroxyl, cyano, (C₁-C₃)-alkyl, (C₁-C₃)-haloalkyl, hydroxy-(C₁-C₃)-alkyl, (C₃-C₆)-cycloalkyl, (C₃-C₆)-halocycloalkyl, hydroxy-(C₃-C₆)-cycloalkyl, (C₁-C₃)-alkoxy, (C₁-C₃)-haloalkoxy, (C₁-C₃)-alkoxycarbonyl, (C₂-C₃) alkenyl, (C₂-C₃)-haloalkenyl, (C₂-C₃) alkynyl, (C₂-C₃)-haloalkynyl, (C₁-C₃)-alkylthio, (C₁-C₃)-alkylsulfinyl, (C₁-C₃)-alkylsulfonyl;
R⁴ hydrogen, halogen, hydroxyl, cyano, (C₁-C₃)-alkyl, (C₁-C₃)-haloalkyl, (C₃-C₄)-halocycloalkyl, (C₁-C₃)-haloalkoxy, (C₂-C₃)-haloalkenyl, (C₂-C₃)-haloalkynyl;
R⁵ hydrogen, halogen, nitro, hydroxyl, cyano, (C₁-C₃)-alkyl, (C₁-C₃)-haloalkyl, hydroxy-(C₁-C₃)-alkyl, (C₃-C₆)-cycloalkyl, (C₃-C₆)-halocycloalkyl, hydroxy-(C₃-C₆)-cycloalkyl, (C₁-C₃)-alkoxy, (C₁-C₃)-haloalkoxy, (C₁-C₃)-alkoxycarbonyl, (C₂-C₃) alkenyl, (C₂-C₃)-haloalkenyl, (C₂-C₃) alkynyl, (C₂-C₃)haloalkynyl, (C₁-C₃)-alkylthio, (C₁-C₃)-alkylsulfinyl, (C₁-C₃)-alkylsulfonyl;
R⁶ hydrogen, halogen, (C₁-C₃)-alkyl, (C₁-C₃)-haloalkyl, (C₁-C₃)-alkoxy, (C₁-C₃)-haloalkoxy;
R⁷ hydrogen, halogen, cyano, or (C₁-C₆)-alkyl, (C₃-C₆)-cycloalkyl, (C₂-C₆)-alkenyl, (C₂-C₆)-alkynyl, (C₁-C₆)-alkoxy, each substituted by m radicals from the group consisting of fluorine, chlorine, bromine, iodine, hydroxyl, cyano and (C₁-C₆)-alkoxy;
R⁸ hydrogen, halogen, (C₁-C₃)-alkyl, (C₁-C₃)-haloalkyl and (C₃-C₆)-cycloalkyl;
R⁹, R¹⁰ each independently hydrogen, halogen, cyano, or (C₁-C₆)-alkyl, (C₁-C₆)-alkoxy, each substituted by m radicals from the group consisting of fluorine, chlorine, bromine, iodine, and cyano; or
R⁹ and R¹⁰ form, together with the carbon atom to which they are bound, a saturated, partially or fully unsaturated three to six-membered ring containing, in addition to this carbon atom, q carbon atoms and n oxygen atoms;
R^{a} (C₁-C₆)-alkyl, (C₃-C₆)-cycloalkyl or phenyl, each of which is substituted by m radicals selected from the group consisting of fluorine, chlorine, bromine, iodine, cyano, hydroxy, and (C₁-C₃)-alkoxy;
R^{b} hydrogen, (C₁-C₃)-alkoxy or R^{a};
R^{h} hydrogen or (C₁-C₆)-alkyl, (C₁-C₂)-alkoxy, (C₃-C₆)-cycloalkyl, (C₂-C₄)-alkenyl, (C₁-C₆)-alkoxycarbonyl-(C₁-C₆)-alkyl, or (C₂-C₄)-alkynyl, each of which is substituted by m radicals selected from the group consisting of fluorine, chlorine, bromine, cyano, CO₂R^{a} and (C₁-C₂)-alkoxy;
Z is a three-, four-, five- or six-membered saturated, partly unsaturated, fully unsaturated or aromatic ring, which is formed from r carbon atoms, n nitrogen atoms, n sulfur atoms and n oxygen atoms, and which is substituted by m radicals selected from the group consisting of fluorine, chlorine, bromine, cyano, (C₁-C₂)-alkoxy, (C₁-C₂)-haloalkoxy; each m is independently 0, 1, 2, 3, 4 or 5;
each n is independently 0, 1 or 2;
q 1, 2, 3, 4 or 5;
r 1, 2, 3, 4, 5, or 6;
including their agriculturally acceptable salts and thioesters, provided the compounds of formula (I) have a carboxyl group; except the compounds methyl 2,3-dihydro-4-phenyl-2-furancarboxylate and methyl 2,3-dihydro-5-methyl-4-phenyl-2-furancarboxylate.

The present invention also provides formulations comprising at least one compound of formula(1) and auxiliaries customary for formulating crop protection agents.

The present invention also provides combinations comprising at least one compound of formula (I) (component A) and at least one further compound selected from the herbicidal compounds B (component B) and safeners C (component C).

The present invention also provides the use of compounds of formula (I) as herbicides, i.e. for controlling undesired vegetation.

The present invention furthermore provides a method for controlling undesired vegetation where a herbicidal effective amount of at least one compound of formula (I) is allowed to act on plants, their seeds and/or their habitat.

If the compounds of formula (I), the herbicidal compounds B and/or the safeners C as described herein are capable of forming geometric isomers, for example E/Z isomers, it is possible to use both, the pure isomers and mixtures thereof, according to the invention.

If the compounds of formula (I), the herbicidal compounds B and/or the safeners C as described herein have one or more centres of chirality and, as a consequence, are present as enantiomers or diastereomers, it is possible to use both, the pure enantiomers and diastereomers and their mixtures, according to the invention.

If the compounds of formula (I), the herbicidal compounds B and/or the safeners C as described herein have ionizable functional groups, they can also be employed in the form of their agriculturally acceptable salts. Suitable are, in general, the salts of those cations and the acid addition salts of those acids whose cations and anions, respectively, have no adverse effect on the activity of the active compounds.

Preferred cations are the ions of the alkali metals, preferably of lithium, sodium and potassium, of the alkaline earth metals, preferably of calcium and magnesium, and of the transition metals, preferably of manganese, copper, zinc and iron, further ammonium and substituted ammonium in which one to four hydrogen atoms are replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, methylammonium, isopropylammonium, dimethylammonium, diethylammonium, diisoprop-ylammonium, trimethylammonium, triethylammonium, tris(isopropyl)ammonium, heptylammo-nium, dodecylammonium, tetradecylammonium, tetramethylammonium, tetraethylammonium, tetrabutylammonium, 2-hydroxyethylammonium (olamine salt), 2-(2-hydroxyeth-1-oxy)eth-1-ylammonium (diglycolamine salt), di(2-hydroxyeth-1-yl)ammonium (diolamine salt), tris(2-hy-droxyethyl)ammonium (trolamine salt), tris(2-hydroxypropyl)ammonium, benzyltrimethylammo-nium, benzyltriethylammonium, N,N,N-trimethylethanolammonium (choline salt), furthermore phosphonium ions, sulfonium ions, preferably tri(C₁-C₄-alkyl)sulfonium, such as trimethyl-sulfonium, and sulfoxonium ions, preferably tri(C₁-C₄-alkyl)sulfoxonium, and finally the salts of polybasic amines such as N,N-bis-(3-aminopropyl)methylamine and diethylenetriamine.

Anions of useful acid addition salts are primarily chloride, bromide, fluoride, iodide, hydrogensul-fate, methylsulfate, sulfate, dihydrogenphosphate, hydrogenphosphate, nitrate, bicarbonate, carbonate, hexafluorosilicate, hexafluorophosphate, benzoate and also the anions of C₁-C₄-al-kanoic acids, preferably formate, acetate, propionate and butyrate.

Compounds of formula (I), herbicidal compounds B and/or safeners C as described herein having a carboxyl group can be employed in the form of the acid, in the form of an agriculturally suitable salt as mentioned above or else in the form of an agriculturally acceptable derivative, for example as amides, such as mono- and di-C₁-C₆-alkylamides or arylamides, as esters, for example as allyl esters, propargyl esters, C₁-C₁₀-alkyl esters, alkoxyalkyl esters, tefuryl ((tetra-hydrofuran-2-yl)methyl) esters and also as thioesters, for example as C₁-C₁₀-alkylthio esters. Preferred mono- and di-C₁-C₆-alkylamides are the methyl and the dimethylamides. Preferred arylamides are, for example, the anilides and the 2-chloroanilides. Preferred alkyl esters are, for example, the methyl, ethyl, propyl, isopropyl, butyl, isobutyl, pentyl, mexyl (1-methylhexyl), meptyl (1-methylheptyl), heptyl, octyl or isooctyl (2-ethylhexyl) esters. Preferred C₁-C₄-alkoxy-C₁-C₄-alkyl esters are the straight-chain or branched C₁-C₄-alkoxy ethyl esters, for example the 2-methoxyethyl, 2-ethoxyethyl, 2-butoxyethyl (butotyl), 2-butoxypropyl or 3-butoxypropyl ester. An example of a straight-chain or branched C₁-C₁₀-alkylthio ester is the ethylthio ester.

The terms used for organic groups in the definition of the variables are, for example the expres-sion “halogen”, collective terms which represent the individual members of these groups of organic units.

The prefix Cr-Cy denotes the number of possible carbon atoms in the particular case. All hydrocarbon chains can be straight-chain or branched.
halogen: fluorine, chlorine, bromine, or iodine, especially fluorine, chlorine or bromine; alkyl and the alkyl moieties of composite groups such as, for example, alkoxy, alkylamino, alkoxycarbonyl: saturated straight-chain or branched hydrocarbon radicals having 1 to 10 carbon atoms, for example C₁-C₁₀-akyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; heptyl, octyl, 2-ethylhexyl and positional isomers thereof; nonyl, decyl and positional isomers thereof;
haloalkyl: straight-chain or branched alkyl groups having 1 to 10 carbon atoms (as mentioned above), where some or all of the hydrogen atoms in these groups are replaced by halogen atoms as mentioned above. In one embodiment, the alkyl groups are substituted at least once or completely by a particular halogen atom, preferably fluorine, chlorine or bromine. In a further embodiment, the alkyl groups are partially or fully halogenated by different halogen atoms; in the case of mixed halogen substitutions, the combination of chlorine and fluorine is preferred. Particular preference is given to (C₁-C₃)-haloalkyl, more preferably (C₁-C₂)-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromo-ethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl and also the alkenyl moieties in composite groups, such as alkenyloxy: unsaturated straight-chain or branched hydrocarbon radicals having 2 to 10 carbon atoms and one double bond in any position. According to the invention, it may be preferred to use small alkenyl groups, such as (C₂-C₄)-alkenyl; on the other hand, it may also be preferred to employ larger alkenyl groups, such as (C₅-C₃)-alkenyl. Examples of alkenyl groups are, for example, C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-di-methyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: alkenyl groups as mentioned above which are partially or fully substituted by fluorine, chlorine, bromine and/or iodine, for example 2-chloroprop-2-en-1-yl, 3-chloroprop-2-en-1-yl, 2,3-dichloroprop-2-en-1-yl, 3,3-dichloroprop-2-en-1-yl, 2,3,3-trichloro-2-en-1-yl, 2,3-dichloro-but-2-en-1-yl, 2-bromoprop-2-en-1-yl, 3-bromoprop-2-en-1-yl, 2,3-dibromoprop-2-en-1-yl, 3,3-dibromoprop-2-en-1-yl, 2,3,3-tribromo-2-en-1-yl or 2,3-dibromobut-2-en-1-yl; alkynyl and the alkynyl moieties in composite groups, such as alkynyloxy: straight-chain or branched hydrocarbon groups having 2 to 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl; haloalkynyl: alkynyl groups as mentioned above which are partially or fully substituted by fluorine, chlorine, bromine and/or iodine, for example 1,1-difluoroprop-2-yn-1-yl, 3-chloroprop-2-yn-1-yl, 3-bromoprop-2-yn-1-yl, 3-iodoprop-2-yn-1-yl, 4-fluorobut-2-yn-1-yl, 4-chlorobut-2-yn-1-yl, 1,1-difluorobut-2-yn-1-yl, 4-iodobut-3-yn-1-yl, 5-fluoropent-3-yn-1-yl, 5-iodopent-4-yn-1-yl, 6-fluorohex-4-yn-1-yl or 6-iodohex-5-yn-1-yl;
cycloalkyl and also the cycloalkyl moieties in composite groups: mono- or bicyclic saturated hydrocarbon groups having 3 to 10, in particular 3 to 6, carbon ring members, for example C₃-C₆-cycloalkyl, such as cyclopropyl, cyclobutyl, cyclopentyl, cyclohexyl, cycloheptyl or cyclooctyl. Examples of bicyclic radicals comprise bicyclo[2.2.1]heptyl, bicyclo[3.1.1]heptyl, bicyclo[2.2.2]octyl and bicyclo[3.2.1]octyl. In this connection, optionally substituted C₃-C₃-cycloalkyl means a cycloalkyl radical having from 3 to 8 carbon atoms, in which at least one hydrogen atom, for example 1, 2, 3, 4 or 5 hydrogen atoms, is/are replaced by substituents which are inert under the conditions of the reaction. Examples of inert substituents are CN, C₁-C₆-alkyl, C₁-C₄-haloalkyl, C₁-C₆-alkoxy, C₃-C₆-cycloalkyl, and C₁-C₄-alkoxy-C₁-C₆-alkyl;
halocycloalkyl and the halocycloalkyl moieties in halocycloalkoxy, halocycloalkylcarbonyl and the like: monocyclic saturated hydrocarbon groups having 3 to 10 carbon ring members (as mentioned above) in which some or all of the hydrogen atoms may be replaced by halogen atoms as mentioned above, in particular fluorine, chlorine and bromine; cycloalkoxy: cycloalkyl groups as mentioned above which are attached via an oxygen; alkoxy and also the alkoxy moieties in composite groups, such as alkoxyalkyl: an alkyl group as defined above which is attached via an oxygen, preferably having 1 to 10, more preferably 2 to 6, carbon atoms. Examples are: methoxy, ethoxy, n-propoxy, 1-methylethoxy, butoxy, 1-methylpropoxy, 2-methylpropoxy or 1,1-dimethylethoxy, and also for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy; haloalkoxy: alkoxy as defined above, where some or all of the hydrogen atoms in these groups are replaced by halogen atoms as described above under haloalkyl, in particular by fluorine, chlorine or bromine. Examples are OCH₂F, OCHF₂, OCF₃, OCH₂Cl, OCHC1₂, OCCl₃, chlorofluo-romethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F5, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F5, OCF₂—C₂F5, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy; and also 5-fluoropentoxy, 5-chloropentoxy, 5-bromopentoxy, 5-iodopentoxy, undecafluoro-pentoxy, 6-fluorohexoxy, 6-chlorohexoxy, 6-bromohexoxy, 6-iodohexoxy or dodecafluoro-hexoxy;
alkylthio: an alkyl group as defined above, which is attached via a sulfur atom to the remainder of the molecule, preferably having 1 to 6, more preferably 1 to 3, e.g. 1 or 2 carbon atoms. C₁-C₂-Alkylthio is methylthio or ethylthio. C₁-C₃-Alkylthio is additionally, for example, n-propylthio or 1-methylethylthio (isopropylthio). C₁-C₅-Alkylthio is additionally, for example, butylthio, 1-methylpropylthio (sec-butylthio), 2-methylpropylthio (isobutylthio), 1,1-dimethylethylthio (tert-butylthio), pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio.
alkylsulfinyl: an alkyl group as defined above, which is attached via S(O) group to the remainder of the molecule, preferably having 1 to 6, more preferably 1 to 3, e.g. 1 or 2 carbon atoms. C₁-C₂-alkylsulfinyl is methylsulfinyl or ethylsulfinyl. C₁-C₃-alkylsulfinyl is additionally, for example, n-propylsulfinyl or 1-methylethylsulfinyl (isopropylsulfinyl). C₁-C₅-alkylsulfinyl is additionally, for example, butylsulfinyl, 1-methylpropylsulfinyl (sec-butylsulfinyl), 2-methylpropylsulfinyl (iso-butylsulfinyl), 1,1-dimethylethylsulfinyl (tert-butylsulfinyl), pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl.
alkysulfonyl: an alkyl group as defined above, which is attached via S(O)₂ group to the remainder of the molecule, preferably having 1 to 6, more preferably 1 to 3, e.g. 1 or 2 carbon atoms. C₁-C₂-alkylsulfonyl is methylsulfonyl or ethylsulfonyl. C₁-C₃-alkylsulfonyl is additionally, for example, n-propylsulfonyl or 1-methylethylsulfonyl (isopropylsulfonyl). C₁-C₅-alkylsulfonyl is additionally, for example, butylsulfonyl, 1-methylpropylsulfonyl (sec-butylsulfonyl), 2-methylpropylsulfonyl (isobutylsulfonyl), 1,1-dimethylethylsulfonyl (tert-butylsulfonyl), pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl.
hydroxyl: OH group which is attached via an O atom;
cyano: CN group which is attached via an C atom;
nitro: NO₂ group which is attached via an N atom.

The preferred embodiments of the invention mentioned herein below have to be understood as being preferred either independently from each other or in combination with one another.

According to particular embodiments of the invention, preference is given to those compounds of formula (I) wherein the variables, either independently of one another or in combination with one another, have the following meanings:
Further preferred compounds according to the invention are compounds of formula (I), wherein R¹ is selected from the group consisting of hydrogen, (C₁-C₃)-alkyl, (C₃-C₄)-cycloalkyl, (C₁-C₃)-haloalkyl, (C₂-C₃)-alkenyl, (C₂-C₃)-alkynyl, (C₁-C₃)-alkoxy-(C₁-C₃)-alkyl.

More preferred compounds according to the invention are compounds of formula (I), wherein R¹ is selected from the group consisting of hydrogen, (C₁-C₃)-alkyl.

Also preferred compounds according to the invention are compounds of formula (I), wherein R¹ is selected from the group consisting of hydrogen, methyl, ethyl.

In particular, R¹ is hydrogen.

Further preferred compounds according to the invention are compounds of formula (I), wherein R² is selected from the group consisting of hydrogen, halogen and (C₁-C₃)-alkyl.

Also preferred compounds according to the invention are compounds of formula (I), wherein R² is selected from the group consisting of hydrogen, fluorine, chlorine and methyl.

In particular, R² is hydrogen.

Further preferred compounds according to the invention are compounds of formula (I), wherein R³ is selected from the group consisting of hydrogen, halogen, hydroxyl, cyano, (C₁-C₃)-alkyl, (C₁-C₃)-haloalkyl, (C₁-C₃)-alkoxy and (C₁-C₃)-haloalkoxy.

Also preferred compounds according to the invention are compounds of formula (I), wherein R³ is selected from the group consisting of hydrogen, halogen, methyl, ethyl, trifluoromethyl, methoxy and trifluoromethoxy.

In particular, R³ is hydrogen or halogen, very particular chlorine or fluorine.

Further preferred compounds according to the invention are compounds of formula (I), wherein R⁴ is selected from the group consisting of hydrogen and halogen.

Also preferred compounds according to the invention are compounds of formula (I), wherein R⁴ is selected from the group consisting of hydrogen, fluorine, chlorine and bromine.

In particular, R⁴ is hydrogen or hydrogen, fluorine or chlorine, very particular hydrogen.

Further preferred compounds according to the invention are compounds of formula (I), wherein R⁵ is selected from the group consisting of hydrogen, halogen, hydroxyl, cyano, (C₁-C₃)-alkyl, (C₁-C₃)-haloalkyl, (C₁-C₃)-alkoxy and (C₁-C₃)-haloalkoxy.

Also preferred compounds according to the invention are compounds of formula (I), wherein R⁵ is selected from the group consisting of hydrogen, halogen, methyl, ethyl, trifluoromethyl, methoxy and trifluoromethoxy.

In particular, R⁵ is hydrogen or halogen, very particular chlorine or fluorine.

Further preferred compounds according to the invention are compounds of formula (I), wherein R⁶ is selected from the group consisting of hydrogen, halogen and (C₁-C₃)-alkyl.

Also preferred compounds according to the invention are compounds of formula (I), wherein R⁶ is selected from the group consisting of hydrogen, fluorine, chlorine and methyl.

In particular, R⁶ is hydrogen.

Further preferred compounds according to the invention are compounds of formula (I), wherein R⁷ is selected from the group consisting of hydrogen, (C₁-C₃)-alkyl, (C₃-C₄)-cycloalkyl, (C₂-C₃)-alkenyl, and (C₁-C₃)-alkoxy, each substituted by m radicals from the group consisting of fluorine, chlorine, and (C₁-C₂)-alkoxy. In this context, m is preferably 0, 1, 2, or 3.

Also preferred compounds according to the invention are compounds of formula (I), wherein R⁷ is selected from the group consisting of hydrogen, (C₁-C₂)-alkyl, cyclopropyl, (C₁-C₂)-haloalkyl, (C₂-C₃)-alkenyl, and (C₁-C₂)-alkoxy.

In particular, R⁷ is hydrogen, methyl, ethyl, chloromethyl, trifluoromethyl, cyclopropyl, ethenyl, and methoxy, very particular hydrogen, methyl, trifluoromethyl.

Further preferred compounds according to the invention are compounds of formula (I), wherein R⁸ is selected from the group consisting of hydrogen, halogen, (C₁-C₃)-alkyl, (C₃-C₄)-cycloalkyl, (C₁-C₂)-haloalkyl.

Also preferred compounds according to the invention are compounds of formula (I), wherein R⁸ is selected from the group consisting of hydrogen, halogen, (C₁-C₂)-alkyl, cyclopropyl, trifluoromethyl.

In particular, R⁸ is hydrogen, fluorine or chlorine, very particular hydrogen.

Further preferred compounds according to the invention are compounds of formula (I), wherein R⁹ and R¹⁰ each independently are selected from the group consisting of hydrogen, halogen, (C₁-C₃)-alkyl, and (C₁-C₃)-haloalkyl.

Further preferred compounds according to the invention are compounds of formula (I), wherein R⁹ and R¹⁰ each independently are selected from the group consisting of hydrogen, fluorine, chlorine, and methyl.

In particular, R⁹ and R¹⁰ are hydrogen.

Preferred compounds of the present invention are compounds of formula (I), wherein the substituents have the following meanings:
R¹ hydrogen or (C₁-C₆)-alkyl, (C₃-C₆)-cycloalkyl, (C₂-C₄)-alkenyl, (C₃-C₆)-cycloalkyl-(C₁-C₃)-alkyl, (C₂-C₄)-alkynyl, each of which is substituted by m radicals selected from the group consisting of fluorine, chlorine, bromine, cyano, Z, CO₂R^{a}, CONR^{b}R^{h}, (C₁-C₂)-alkoxy, (C₁-C₂)-haloalkoxy, (C₁-C₃)-alkylthio, (C₁-C₃)-alkylsulfinyl, (C₁-C₃)-alkylsulfonyl, phenylthio, phenylsulfinyl, and phenylsulfonyl;
R² hydrogen;
R³ halogen, cyano, (C₁-C₃)-alkyl, (C₁-C₃)-haloalkyl, (C₁-C₃)-haloalkoxy, preferably fluorine or chlorine;
R⁴ hydrogen or halogen, preferably hydrogen;
R⁵ halogen, cyano, (C₁-C₃)-alkyl, (C₁-C₃)-haloalkyl, (C₁-C₃)-haloalkoxy, preferably fluorine or chlorine;
R⁶ hydrogen;
R⁷ hydrogen, fluorine, cyano, or (C₁-C₆)-alkyl, (C₃-C₆)-cycloalkyl, (C₂-C₆)-alkenyl, (C₂-C₆)-alkynyl, (C₁-C₆)-alkoxy, each substituted by m radicals from the group consisting of fluorine, chlorine, bromine, iodine, hydroxyl, cyano and (C₁-C₆)-alkoxy;
R⁸ hydrogen, halogen, (C₁-C₃)-alkyl, (C₁-C₃)-haloalkyl, preferably hydrogen;
R⁹, R¹⁰ each independently hydrogen, halogen, cyano, or (C₁-C₆)-alkyl, (C₁-C₆)-alkoxy, each substituted by m radicals from the group consisting of fluorine, chlorine, bromine, iodine, and cyano; or
R⁹ and R¹⁰ form, together with the carbon atom to which they are bound, a saturated, partially or fully unsaturated three to six-membered ring containing, in addition to this carbon atom, q carbon atoms and n oxygen atoms;
Z a three-, four-, five- or six-membered saturated, partly unsaturated, fully unsaturated or aromatic ring, which is formed from r carbon atoms, n nitrogen atoms, n sulfur atoms and n oxygen atoms, and which is substituted by m radicals selected from the group consisting of fluorine, chlorine, bromine, cyano, (C₁-C₂)-alkoxy, (C₁-C₂)-haloalkoxy;
R^{a} (C₁-C₆)-alkyl (C₃-C₆)-cycloalkyl or phenyl, each of which is substituted by m radicals selected from the group consisting of fluorine, chlorine, bromine, iodine, cyano and hydroxy;
R^{b} hydrogen, (C₁-C₃)-alkoxy or R^{a};
R^{h} hydrogen or (C₁-C₆)-alkyl, (C₃-C₆)-cycloalkyl, (C₂-C₄)-alkenyl, (C₁-C₆)-alkoxycarbonyl-(C₁-C₆)-alkyl, or (C₂-C₄)-alkynyl each of which is substituted by m radicals selected from the group consisting of fluorine, chlorine, bromine, cyano and (C₁-C₂)-alkoxy;
each m is independently 0, 1, 2, 3, 4 or 5;
each n is independently 0, 1 or 2;
r 2, 3, 4, 5 or 6;
q 1, 2, 3, 4 or 5.

Further preferred compounds of the present invention are compounds of formula (I), wherein the substituents have the following meanings:
R¹ hydrogen or (C₁-C₆)-alkyl, (C₃-C₆)-cycloalkyl, (C₂-C₄)-alkenyl, (C₃-C₆)-cycloalkyl-(C₁-C₃)-alkyl, (C₂-C₄)-alkynyl, each of which is substituted by m radicals selected from the group consisting of fluorine, chlorine, bromine, cyano, Z;
R² hydrogen;
R³ halogen, cyano, (C₁-C₃)-alkyl, (C₁-C₃)-haloalkyl, (C₁-C₃)-haloalkoxy, preferably fluorine or chlorine;
R⁴ hydrogen or halogen, preferably hydrogen;
R⁵ halogen, cyano, (C₁-C₃)-alkyl, (C₁-C₃)-haloalkyl, (C₁-C₃)-haloalkoxy, preferably fluorine or chlorine;
R⁶ hydrogen;
R⁷ hydrogen, (C₁-C₆)-alkyl, (C₃-C₆)-cycloalkyl, (C₂-C₆)-alkenyl, (C₂-C₆)-alkynyl, (C₁-C₆)-alkoxy, each substituted by m radicals from the group consisting of fluorine, chlorine, bromine, iodine, hydroxyl, cyano and (C₁-C₆)-alkoxy;
R⁸ hydrogen, halogen, (C₁-C₃)-alkyl, (C₁-C₃)-haloalkyl, preferably hydrogen;
R⁹, R¹⁰ hydrogen;
Z a five- or six-membered saturated, partly unsaturated, fully unsaturated or aromatic ring, which is formed from r carbon atoms, n nitrogen atoms, n sulfur atoms and n oxygen atoms, and which is substituted by m radicals selected from the group consisting of fluorine, chlorine, bromine, cyano, (C₁-C₂)-alkoxy, (C₁-C₂)-haloalkoxy
each m is independently 0, 1, 2, 3, 4 or 5;
each n is independently 0, 1 or 2;
r 3, 4, 5 or 6.

Further preferred compounds of the present invention are compounds of formula (I), wherein the substituents have the following meanings:
R¹ hydrogen or (C₁-C₆)-alkyl;
R² hydrogen;
R³ halogen, cyano, (C₁-C₃)-alkyl, (C₁-C₃)-haloalkyl, (C₁-C₃)-haloalkoxy, preferably fluorine or chlorine;
R⁴ hydrogen or halogen, preferably hydrogen;
R⁵ halogen, cyano, (C₁-C₃)-alkyl, (C₁-C₃)-haloalkyl, (C₁-C₃)-haloalkoxy, preferably fluorine or chlorine;
R⁶ hydrogen;
R⁷ (C₁-C₆)-alkyl, (C₃-C₆)-cycloalkyl, (C₂-C₆)-alkenyl, (C₂-C₆)-alkynyl, (C₁-C₆)-alkoxy, each substituted by m radicals from the group consisting of fluorine, chlorine, bromine, iodine, hydroxyl, cyano and (C₁-C₆)-alkoxy;
R⁸ hydrogen, halogen, (C₁-C₃)-alkyl, (C₁-C₃)-haloalkyl, preferably hydrogen;
R⁹, R¹⁰ each independently hydrogen, halogen, cyano, or (C₁-C₆)-alkyl, (C₁-C₆)-alkoxy, each substituted by m radicals from the group consisting of fluorine, chlorine, bromine, iodine, and cyano; or
R⁹ and R¹⁰ form, together with the carbon atom to which they are bound, a saturated, partially or fully unsaturated three to six-membered ring containing, in addition to this carbon atom, q carbon atoms and n oxygen atoms;
each m is independently 0, 1, 2, 3, 4 or 5;
each n is independently 0, 1 or 2;
q 1, 2, 3, 4 or 5.

Further preferred compounds of the present invention are compounds of formula (I), wherein the substituents have the following meanings:
R¹ hydrogen or (C₁-C₆)-alkyl;
R² hydrogen;
R³ halogen, cyano, (C₁-C₃)-alkyl, (C₁-C₃)-haloalkyl, (C₁-C₃)-haloalkoxy, preferably fluorine or chlorine;
R⁴ hydrogen or halogen, preferably hydrogen;
R⁵ halogen, cyano, (C₁-C₃)-alkyl, (C₁-C₃)-haloalkyl, (C₁-C₃)-haloalkoxy, preferably fluorine or chlorine;
R⁶ hydrogen;
R⁷ hydrogen, (C₁-C₆)-alkyl, (C₃-C₆)-cycloalkyl, (C₂-C₆)-alkenyl, (C₂-C₆)-alkynyl, (C₁-C₆)-alkoxy, each substituted by m radicals from the group consisting of fluorine, chlorine, bromine, iodine, hydroxyl, cyano and (C₁-C₆)-alkoxy;
R⁸ hydrogen, halogen, (C₁-C₃)-alkyl, (C₁-C₃)-haloalkyl, preferably hydrogen;
R⁹, R¹⁰ hydrogen;
m 0, 1, 2, 3, 4 or 5.

Further preferred compounds of the present invention are compounds of formula (I), wherein the substituents have the following meanings:
R¹ hydrogen or (C₁-C₆)-alkyl;
R² hydrogen;
R³ halogen, cyano, (C₁-C₃)-alkyl, (C₁-C₃)-haloalkyl, (C₁-C₃)-haloalkoxy, preferably fluorine or chlorine;
R⁴ hydrogen or halogen, preferably hydrogen;
R⁵ halogen, cyano, (C₁-C₃)-alkyl, (C₁-C₃)-haloalkyl, (C₁-C₃)-haloalkoxy, preferably fluorine or chlorine;
R⁶ hydrogen;
R⁷ hydrogen, (C₁-C₂)-alkyl, (C₁-C₂)-haloalkyl, (C₃-C₆)-cycloalkyl, (C₂-C₆)-alkenyl, (C₂-C₆)-alkynyl, (C₁-C₆)-alkoxy, preferably hydrogen, methyl, trifluoromethyl;
R⁸ hydrogen, halogen, (C₁-C₃)-alkyl, (C₁-C₃)-haloalkyl, preferably hydrogen;
R⁹, R¹⁰ hydrogen.

Further preferred embodiments of compounds of formula (I) are compounds I.I to I.IV, wherein
(I.I): R¹ is hydrogen:

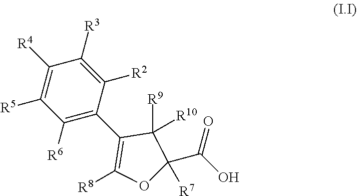

(I.II): R¹ is methyl:

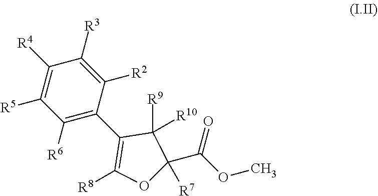

(I.III): R¹ is ethyl:

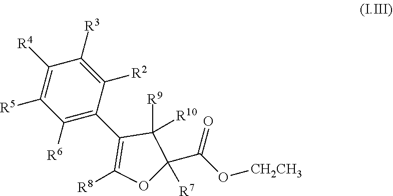

(I.IV): R¹ is CH₂Z:

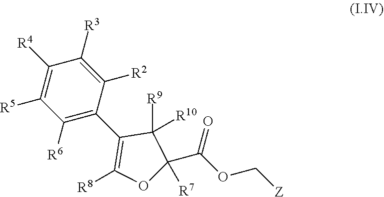

Compounds of formula (I.I.a.) wherein R¹, R², R⁶, R⁸, R⁹ and R¹⁰ are hydrogen are also particularly preferred:

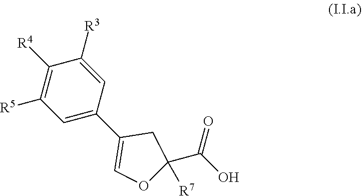

Compounds of formula (I.I.b.) wherein R¹, R², R⁴, R⁶, R⁸, R⁹ and R¹⁰ are hydrogen are also particularly preferred:

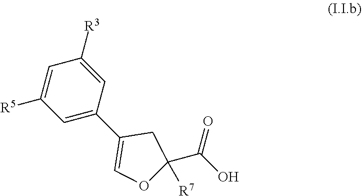

Compounds of formula (I.II.a.) wherein R², R⁶, R⁸, R⁹ and R¹⁰ are hydrogen, R¹ is methyl are particularly preferred:

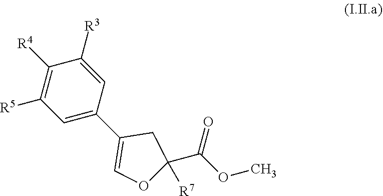

Compounds of formula (I.II.b.) wherein R², R⁴, R⁶, R⁸, R⁹ and R¹⁰ are hydrogen, R¹ is methyl are particularly preferred:

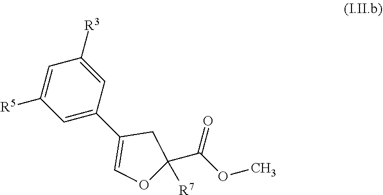

Compounds of formula (I.III.a.) wherein R², R⁶, R⁸, R⁹ and R¹⁰ are hydrogen, R¹ is ethyl are particularly preferred:

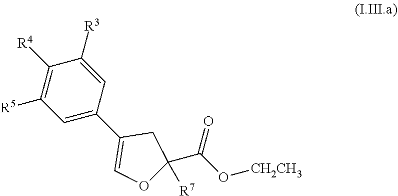

Compounds of formula (I.III.a.) wherein R², R⁴, R⁶, R⁸, R⁹ and R¹⁰ are hydrogen, R¹ is ethyl are particularly preferred are particularly preferred:

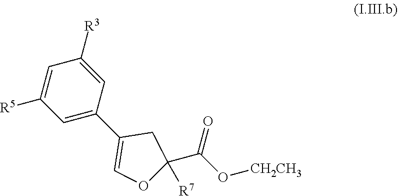

Compounds of formula (I.IV.a.) wherein R², R⁶, R⁸, R⁹ and R¹⁰ are hydrogen, R¹ is CH₂Z are particularly preferred:

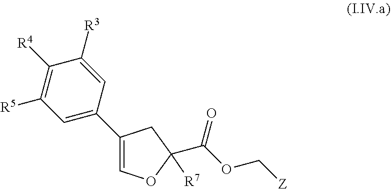

Compounds of formula (I.IV.b.) wherein R², R⁴, R⁶, R⁸, R⁹ and R¹⁰ are hydrogen, R¹ is CH₂Z are particularly preferred are particularly preferred:

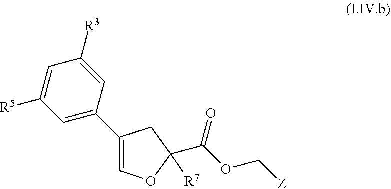

In the context of the present invention, compounds wherein R¹, R², R⁶, R⁸, R⁹ and R¹⁰ are hydrogen (compounds I.I.a) and R³, R⁴, R⁵ and R⁷ have the meanings as defined lines in 1 to 1155 of Table 1 below, are particularly preferred.

TABLE 1
In Table 1,

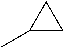

means cyclopropyl.

| Cpd. | R³ | R⁴ | R⁵ | R⁷ |
|---|---|---|---|---|
| 1. | H | H | H | CH₃ |
| 2. | F | H | H | CH₃ |
| 3. | Cl | H | H | CH₃ |
| 4. | Br | H | H | CH₃ |
| 5. | I | H | H | CH₃ |
| 6. | CN | H | H | CH₃ |
| 7. | CH₃ | H | H | CH₃ |
| 8. | CF₃ | H | H | CH₃ |
| 9. | OCH₃ | H | H | CH₃ |
| 10. | OCF₃ | H | H | CH₃ |
| 11. | CH₂CH₃ | H | H | CH₃ |
| 12. | H | F | H | CH₃ |
| 13. | F | F | H | CH₃ |
| 14. | Cl | F | H | CH₃ |
| 15. | Br | F | H | CH₃ |
| 16. | I | F | H | CH₃ |
| 17. | CN | F | H | CH₃ |
| 18. | CH₃ | F | H | CH₃ |
| 19. | CF₃ | F | H | CH₃ |
| 20. | OCH₃ | F | H | CH₃ |
| 21. | OCF₃ | F | H | CH₃ |
| 22. | CH₂CH₃ | F | H | CH₃ |
| 23. | H | Cl | H | CH₃ |
| 24. | F | Cl | H | CH₃ |
| 25. | Cl | Cl | H | CH₃ |
| 26. | Br | Cl | H | CH₃ |
| 27. | I | Cl | H | CH₃ |
| 28. | CN | Cl | H | CH₃ |
| 29. | CH₃ | Cl | H | CH₃ |
| 30. | CF₃ | Cl | H | CH₃ |
| 31. | OCH₃ | Cl | H | CH₃ |
| 32. | OCF₃ | Cl | H | CH₃ |
| 33. | CH₂CH₃ | Cl | H | CH₃ |
| 34. | H | H | F | CH₃ |
| 35. | F | H | F | CH₃ |
| 36. | Cl | H | F | CH₃ |
| 37. | Br | H | F | CH₃ |
| 38. | I | H | F | CH₃ |
| 39. | CN | H | F | CH₃ |
| 40. | CH₃ | H | F | CH₃ |
| 41. | CF₃ | H | F | CH₃ |
| 42. | OCH₃ | H | F | CH₃ |
| 43. | OCF₃ | H | F | CH₃ |
| 44. | CH₂CH₃ | H | F | CH₃ |
| 45. | H | F | F | CH₃ |
| 46. | F | F | F | CH₃ |
| 47. | Cl | F | F | CH₃ |
| 48. | Br | F | F | CH₃ |
| 49. | I | F | F | CH₃ |
| 50. | CN | F | F | CH₃ |
| 51. | CH₃ | F | F | CH₃ |
| 52. | CF₃ | F | F | CH₃ |
| 53. | OCH₃ | F | F | CH₃ |
| 54. | OCF₃ | F | F | CH₃ |
| 55. | CH₂CH₃ | F | F | CH₃ |
| 56. | H | Cl | F | CH₃ |
| 57. | F | Cl | F | CH₃ |
| 58. | Cl | Cl | F | CH₃ |
| 59. | Br | Cl | F | CH₃ |
| 60. | I | Cl | F | CH₃ |
| 61. | CN | Cl | F | CH₃ |
| 62. | CH₃ | Cl | F | CH₃ |
| 63. | CF₃ | Cl | F | CH₃ |
| 64. | OCH₃ | Cl | F | CH₃ |
| 65. | OCF₃ | Cl | F | CH₃ |
| 66. | CH₂CH₃ | Cl | F | CH₃ |
| 67. | H | H | Cl | CH₃ |
| 68. | F | H | Cl | CH₃ |
| 69. | Cl | H | Cl | CH₃ |
| 70. | Br | H | Cl | CH₃ |
| 71. | I | H | Cl | CH₃ |
| 72. | CN | H | Cl | CH₃ |
| 73. | CH₃ | H | Cl | CH₃ |
| 74. | CF₃ | H | Cl | CH₃ |
| 75. | OCH₃ | H | Cl | CH₃ |
| 76. | OCF₃ | H | Cl | CH₃ |
| 77. | CH₂CH₃ | H | Cl | CH₃ |
| 78. | H | F | Cl | CH₃ |
| 79. | F | F | Cl | CH₃ |
| 80. | Cl | F | Cl | CH₃ |
| 81. | Br | F | Cl | CH₃ |
| 82. | I | F | Cl | CH₃ |
| 83. | CN | F | Cl | CH₃ |
| 84. | CH₃ | F | Cl | CH₃ |
| 85. | CF₃ | F | Cl | CH₃ |
| 86. | OCH₃ | F | Cl | CH₃ |
| 87. | OCF₃ | F | Cl | CH₃ |
| 88. | CH₂CH₃ | F | Cl | CH₃ |
| 89. | H | Cl | Cl | CH₃ |
| 90. | F | Cl | Cl | CH₃ |
| 91. | Cl | Cl | Cl | CH₃ |
| 92. | Br | Cl | Cl | CH₃ |
| 93. | I | Cl | Cl | CH₃ |
| 94. | CN | Cl | Cl | CH₃ |
| 95. | CH₃ | Cl | Cl | CH₃ |
| 96. | CF₃ | Cl | Cl | CH₃ |
| 97. | OCH₃ | Cl | Cl | CH₃ |
| 98. | OCF₃ | Cl | Cl | CH₃ |
| 99. | CH₂CH₃ | Cl | Cl | CH₃ |
| 100. | H | H | Br | CH₃ |
| 101. | F | H | Br | CH₃ |
| 102. | Cl | H | Br | CH₃ |
| 103. | Br | H | Br | CH₃ |
| 104. | I | H | Br | CH₃ |
| 105. | CN | H | Br | CH₃ |
| 106. | CH₃ | H | Br | CH₃ |
| 107. | CF₃ | H | Br | CH₃ |
| 108. | OCH₃ | H | Br | CH₃ |
| 109. | OCF₃ | H | Br | CH₃ |
| 110. | CH₂CH₃ | H | Br | CH₃ |
| 111. | H | F | Br | CH₃ |
| 112. | F | F | Br | CH₃ |
| 113. | Cl | F | Br | CH₃ |
| 114. | Br | F | Br | CH₃ |
| 115. | I | F | Br | CH₃ |
| 116. | CN | F | Br | CH₃ |
| 117. | CH₃ | F | Br | CH₃ |
| 118. | CF₃ | F | Br | CH₃ |
| 119. | OCH₃ | F | Br | CH₃ |
| 120. | OCF₃ | F | Br | CH₃ |
| 121. | CH₂CH₃ | F | Br | CH₃ |
| 122. | H | Cl | Br | CH₃ |
| 123. | F | Cl | Br | CH₃ |
| 124. | Cl | Cl | Br | CH₃ |
| 125. | Br | Cl | Br | CH₃ |
| 126. | I | Cl | Br | CH₃ |
| 127. | CN | Cl | Br | CH₃ |
| 128. | CH₃ | Cl | Br | CH₃ |
| 129. | CF₃ | Cl | Br | CH₃ |
| 130. | OCH₃ | Cl | Br | CH₃ |
| 131. | OCF₃ | Cl | Br | CH₃ |
| 132. | CH₂CH₃ | Cl | Br | CH₃ |
| 133. | H | H | I | CH₃ |
| 134. | F | H | I | CH₃ |
| 135. | Cl | H | I | CH₃ |
| 136. | Br | H | I | CH₃ |
| 137. | I | H | I | CH₃ |
| 138. | CN | H | I | CH₃ |
| 139. | CH₃ | H | I | CH₃ |
| 140. | CF₃ | H | I | CH₃ |
| 141. | OCH₃ | H | I | CH₃ |
| 142. | OCF₃ | H | I | CH₃ |
| 143. | CH₂CH₃ | H | I | CH₃ |
| 144. | H | F | I | CH₃ |
| 145. | F | F | I | CH₃ |
| 146. | Cl | F | I | CH₃ |
| 147. | Br | F | I | CH₃ |
| 148. | I | F | I | CH₃ |
| 149. | CN | F | I | CH₃ |
| 150. | CH₃ | F | I | CH₃ |
| 151. | CF₃ | F | I | CH₃ |
| 152. | OCH₃ | F | I | CH₃ |
| 153. | OCF₃ | F | I | CH₃ |
| 154. | CH₂CH₃ | F | I | CH₃ |
| 155. | H | Cl | I | CH₃ |
| 156. | F | Cl | I | CH₃ |
| 157. | Cl | Cl | I | CH₃ |
| 158. | Br | Cl | I | CH₃ |
| 159. | I | Cl | I | CH₃ |
| 160. | CN | Cl | I | CH₃ |
| 161. | CH₃ | Cl | I | CH₃ |
| 162. | CF₃ | Cl | I | CH₃ |
| 163. | OCH₃ | Cl | I | CH₃ |
| 164. | OCF₃ | Cl | I | CH₃ |
| 165. | CH₂CH₃ | Cl | I | CH₃ |
| 166. | H | H | CN | CH₃ |
| 167. | F | H | CN | CH₃ |
| 168. | Cl | H | CN | CH₃ |
| 169. | Br | H | CN | CH₃ |
| 170. | I | H | CN | CH₃ |
| 171. | CN | H | CN | CH₃ |
| 172. | CH₃ | H | CN | CH₃ |
| 173. | CF₃ | H | CN | CH₃ |
| 174. | OCH₃ | H | CN | CH₃ |
| 175. | OCF₃ | H | CN | CH₃ |
| 176. | CH₂CH₃ | H | CN | CH₃ |
| 177. | H | F | CN | CH₃ |
| 178. | F | F | CN | CH₃ |
| 179. | Cl | F | CN | CH₃ |
| 180. | Br | F | CN | CH₃ |
| 181. | I | F | CN | CH₃ |
| 182. | CN | F | CN | CH₃ |
| 183. | CH₃ | F | CN | CH₃ |
| 184. | CF₃ | F | CN | CH₃ |
| 185. | OCH₃ | F | CN | CH₃ |
| 186. | OCF₃ | F | CN | CH₃ |
| 187. | CH₂CH₃ | F | CN | CH₃ |
| 188. | H | Cl | CN | CH₃ |
| 189. | F | Cl | CN | CH₃ |
| 190. | Cl | Cl | CN | CH₃ |
| 191. | Br | Cl | CN | CH₃ |
| 192. | I | Cl | CN | CH₃ |
| 193. | CN | Cl | CN | CH₃ |
| 194. | CH₃ | Cl | CN | CH₃ |
| 195. | CF₃ | Cl | CN | CH₃ |
| 196. | OCH₃ | Cl | CN | CH₃ |
| 197. | OCF₃ | Cl | CN | CH₃ |
| 198. | CH₂CH₃ | Cl | CN | CH₃ |
| 199. | H | H | CH₃ | CH₃ |
| 200. | F | H | CH₃ | CH₃ |
| 201. | Cl | H | CH₃ | CH₃ |
| 202. | Br | H | CH₃ | CH₃ |
| 203. | I | H | CH₃ | CH₃ |
| 204. | CN | H | CH₃ | CH₃ |
| 205. | CH₃ | H | CH₃ | CH₃ |
| 206. | CF₃ | H | CH₃ | CH₃ |
| 207. | OCH₃ | H | CH₃ | CH₃ |
| 208. | OCF₃ | H | CH₃ | CH₃ |
| 209. | CH₂CH₃ | H | CH₃ | CH₃ |
| 210. | H | F | CH₃ | CH₃ |
| 211. | F | F | CH₃ | CH₃ |
| 212. | Cl | F | CH₃ | CH₃ |
| 213. | Br | F | CH₃ | CH₃ |
| 214. | I | F | CH₃ | CH₃ |
| 215. | CN | F | CH₃ | CH₃ |
| 216. | CH₃ | F | CH₃ | CH₃ |
| 217. | CF₃ | F | CH₃ | CH₃ |
| 218. | OCH₃ | F | CH₃ | CH₃ |
| 219. | OCF₃ | F | CH₃ | CH₃ |
| 220. | CH₂CH₃ | F | CH₃ | CH₃ |
| 221. | H | Cl | CH₃ | CH₃ |
| 222. | F | Cl | CH₃ | CH₃ |
| 223. | Cl | Cl | CH₃ | CH₃ |
| 224. | Br | Cl | CH₃ | CH₃ |
| 225. | I | Cl | CH₃ | CH₃ |
| 226. | CN | Cl | CH₃ | CH₃ |
| 227. | CH₃ | Cl | CH₃ | CH₃ |
| 228. | CF₃ | Cl | CH₃ | CH₃ |
| 229. | OCH₃ | Cl | CH₃ | CH₃ |
| 230. | OCF₃ | Cl | CH₃ | CH₃ |
| 231. | CH₂CH₃ | Cl | CH₃ | CH₃ |
| 232. | H | H | H | CHCH₂ |
| 233. | F | H | H | CHCH₂ |
| 234. | Cl | H | H | CHCH₂ |
| 235. | Br | H | H | CHCH₂ |
| 236. | I | H | H | CHCH₂ |
| 237. | CN | H | H | CHCH₂ |
| 238. | CH₃ | H | H | CHCH₂ |
| 239. | CF₃ | H | H | CHCH₂ |
| 240. | OCH₃ | H | H | CHCH₂ |
| 241. | OCF₃ | H | H | CHCH₂ |
| 242. | CH₂CH₃ | H | H | CHCH₂ |
| 243. | H | F | H | CHCH₂ |
| 244. | F | F | H | CHCH₂ |
| 245. | Cl | F | H | CHCH₂ |
| 246. | Br | F | H | CHCH₂ |
| 247. | I | F | H | CHCH₂ |
| 248. | CN | F | H | CHCH₂ |
| 249. | CH₃ | F | H | CHCH₂ |
| 250. | CF₃ | F | H | CHCH₂ |
| 251. | OCH₃ | F | H | CHCH₂ |
| 252. | OCF₃ | F | H | CHCH₂ |
| 253. | CH₂CH₃ | F | H | CHCH₂ |
| 254. | H | Cl | H | CHCH₂ |
| 255. | F | Cl | H | CHCH₂ |
| 256. | Cl | Cl | H | CHCH₂ |
| 257. | Br | Cl | H | CHCH₂ |
| 258. | I | Cl | H | CHCH₂ |
| 259. | CN | Cl | H | CHCH₂ |
| 260. | CH₃ | Cl | H | CHCH₂ |
| 261. | CF₃ | Cl | H | CHCH₂ |
| 262. | OCH₃ | Cl | H | CHCH₂ |
| 263. | OCF₃ | Cl | H | CHCH₂ |
| 264. | CH₂CH₃ | Cl | H | CHCH₂ |
| 265. | H | H | F | CHCH₂ |
| 266. | F | H | F | CHCH₂ |
| 267. | Cl | H | F | CHCH₂ |
| 268. | Br | H | F | CHCH₂ |
| 269. | I | H | F | CHCH₂ |
| 270. | CN | H | F | CHCH₂ |
| 271. | CH₃ | H | F | CHCH₂ |
| 272. | CF₃ | H | F | CHCH₂ |
| 273. | OCH₃ | H | F | CHCH₂ |
| 274. | OCF₃ | H | F | CHCH₂ |
| 275. | CH₂CH₃ | H | F | CHCH₂ |
| 276. | H | F | F | CHCH₂ |
| 277. | F | F | F | CHCH₂ |
| 278. | Cl | F | F | CHCH₂ |
| 279. | Br | F | F | CHCH₂ |
| 280. | I | F | F | CHCH₂ |
| 281. | CN | F | F | CHCH₂ |
| 282. | CH₃ | F | F | CHCH₂ |
| 283. | CF₃ | F | F | CHCH₂ |
| 284. | OCH₃ | F | F | CHCH₂ |
| 285. | OCF₃ | F | F | CHCH₂ |
| 286. | CH₂CH₃ | F | F | CHCH₂ |
| 287. | H | Cl | F | CHCH₂ |
| 288. | F | Cl | F | CHCH₂ |
| 289. | Cl | Cl | F | CHCH₂ |
| 290. | Br | Cl | F | CHCH₂ |
| 291. | I | Cl | F | CHCH₂ |
| 292. | CN | Cl | F | CHCH₂ |
| 293. | CH₃ | Cl | F | CHCH₂ |
| 294. | CF₃ | Cl | F | CHCH₂ |
| 295. | OCH₃ | Cl | F | CHCH₂ |
| 296. | OCF₃ | Cl | F | CHCH₂ |
| 297. | CH₂CH₃ | Cl | F | CHCH₂ |
| 298. | H | H | Cl | CHCH₂ |
| 299. | F | H | Cl | CHCH₂ |
| 300. | Cl | H | Cl | CHCH₂ |
| 301. | Br | H | Cl | CHCH₂ |
| 302. | I | H | Cl | CHCH₂ |
| 303. | CN | H | Cl | CHCH₂ |
| 304. | CH₃ | H | Cl | CHCH₂ |
| 305. | CF₃ | H | Cl | CHCH₂ |
| 306. | OCH₃ | H | Cl | CHCH₂ |
| 307. | OCF₃ | H | Cl | CHCH₂ |
| 308. | CH₂CH₃ | H | Cl | CHCH₂ |
| 309. | H | F | Cl | CHCH₂ |
| 310. | F | F | Cl | CHCH₂ |
| 311. | Cl | F | Cl | CHCH₂ |
| 312. | Br | F | Cl | CHCH₂ |
| 313. | I | F | Cl | CHCH₂ |
| 314. | CN | F | Cl | CHCH₂ |
| 315. | CH₃ | F | Cl | CHCH₂ |
| 316. | CF₃ | F | Cl | CHCH₂ |
| 317. | OCH₃ | F | Cl | CHCH₂ |
| 318. | OCF₃ | F | Cl | CHCH₂ |
| 319. | CH₂CH₃ | F | Cl | CHCH₂ |
| 320. | H | Cl | Cl | CHCH₂ |
| 321. | F | Cl | Cl | CHCH₂ |
| 322. | Cl | Cl | Cl | CHCH₂ |
| 323. | Br | Cl | Cl | CHCH₂ |
| 324. | I | Cl | Cl | CHCH₂ |
| 325. | CN | Cl | Cl | CHCH₂ |
| 326. | CH₃ | Cl | Cl | CHCH₂ |
| 327. | CF₃ | Cl | Cl | CHCH₂ |
| 328. | OCH₃ | Cl | Cl | CHCH₂ |
| 329. | OCF₃ | Cl | Cl | CHCH₂ |
| 330. | CH₂CH₃ | Cl | Cl | CHCH₂ |
| 331. | H | H | Br | CHCH₂ |
| 332. | F | H | Br | CHCH₂ |
| 333. | Cl | H | Br | CHCH₂ |
| 334. | Br | H | Br | CHCH₂ |
| 335. | I | H | Br | CHCH₂ |
| 336. | CN | H | Br | CHCH₂ |
| 337. | CH₃ | H | Br | CHCH₂ |
| 338. | CF₃ | H | Br | CHCH₂ |
| 339. | OCH₃ | H | Br | CHCH₂ |
| 340. | OCF₃ | H | Br | CHCH₂ |
| 341. | CH₂CH₃ | H | Br | CHCH₂ |
| 342. | H | F | Br | CHCH₂ |
| 343. | F | F | Br | CHCH₂ |
| 344. | Cl | F | Br | CHCH₂ |
| 345. | Br | F | Br | CHCH₂ |
| 346. | I | F | Br | CHCH₂ |
| 347. | CN | F | Br | CHCH₂ |
| 348. | CH₃ | F | Br | CHCH₂ |
| 349. | CF₃ | F | Br | CHCH₂ |
| 350. | OCH₃ | F | Br | CHCH₂ |
| 351. | OCF₃ | F | Br | CHCH₂ |
| 352. | CH₂CH₃ | F | Br | CHCH₂ |
| 353. | H | Cl | Br | CHCH₂ |
| 354. | F | Cl | Br | CHCH₂ |
| 355. | Cl | Cl | Br | CHCH₂ |
| 356. | Br | Cl | Br | CHCH₂ |
| 357. | I | Cl | Br | CHCH₂ |
| 358. | CN | Cl | Br | CHCH₂ |
| 359. | CH₃ | Cl | Br | CHCH₂ |
| 360. | CF₃ | Cl | Br | CHCH₂ |
| 361. | OCH₃ | Cl | Br | CHCH₂ |
| 362. | OCF₃ | Cl | Br | CHCH₂ |
| 363. | CH₂CH₃ | Cl | Br | CHCH₂ |
| 364. | H | H | I | CHCH₂ |
| 365. | F | H | I | CHCH₂ |
| 366. | Cl | H | I | CHCH₂ |
| 367. | Br | H | I | CHCH₂ |
| 368. | I | H | I | CHCH₂ |
| 369. | CN | H | I | CHCH₂ |
| 370. | CH₃ | H | I | CHCH₂ |
| 371. | CF₃ | H | I | CHCH₂ |
| 372. | OCH₃ | H | I | CHCH₂ |
| 373. | OCF₃ | H | I | CHCH₂ |
| 374. | CH₂CH₃ | H | I | CHCH₂ |
| 375. | H | F | I | CHCH₂ |
| 376. | F | F | I | CHCH₂ |
| 377. | Cl | F | I | CHCH₂ |
| 378. | Br | F | I | CHCH₂ |
| 379. | I | F | I | CHCH₂ |
| 380. | CN | F | I | CHCH₂ |
| 381. | CH₃ | F | I | CHCH₂ |
| 382. | CF₃ | F | I | CHCH₂ |
| 383. | OCH₃ | F | I | CHCH₂ |
| 384. | OCF₃ | F | I | CHCH₂ |
| 385. | CH₂CH₃ | F | I | CHCH₂ |
| 386. | H | Cl | I | CHCH₂ |
| 387. | F | Cl | I | CHCH₂ |
| 388. | Cl | Cl | I | CHCH₂ |
| 389. | Br | Cl | I | CHCH₂ |
| 390. | I | Cl | I | CHCH₂ |
| 391. | CN | Cl | I | CHCH₂ |
| 392. | CH₃ | Cl | I | CHCH₂ |
| 393. | CF₃ | Cl | I | CHCH₂ |
| 394. | OCH₃ | Cl | I | CHCH₂ |
| 395. | OCF₃ | Cl | I | CHCH₂ |
| 396. | CH₂CH₃ | Cl | I | CHCH₂ |
| 397. | H | H | CN | CHCH₂ |
| 398. | F | H | CN | CHCH₂ |
| 399. | Cl | H | CN | CHCH₂ |
| 400. | Br | H | CN | CHCH₂ |
| 401. | I | H | CN | CHCH₂ |
| 402. | CN | H | CN | CHCH₂ |
| 403. | CH₃ | H | CN | CHCH₂ |
| 404. | CF₃ | H | CN | CHCH₂ |
| 405. | OCH₃ | H | CN | CHCH₂ |
| 406. | OCF₃ | H | CN | CHCH₂ |
| 407. | CH₂CH₃ | H | CN | CHCH₂ |
| 408. | H | F | CN | CHCH₂ |
| 409. | F | F | CN | CHCH₂ |
| 410. | Cl | F | CN | CHCH₂ |
| 411. | Br | F | CN | CHCH₂ |
| 412. | I | F | CN | CHCH₂ |
| 413. | CN | F | CN | CHCH₂ |
| 414. | CH₃ | F | CN | CHCH₂ |
| 415. | CF₃ | F | CN | CHCH₂ |
| 416. | OCH₃ | F | CN | CHCH₂ |
| 417. | OCF₃ | F | CN | CHCH₂ |
| 418. | CH₂CH₃ | F | CN | CHCH₂ |
| 419. | H | Cl | CN | CHCH₂ |
| 420. | F | Cl | CN | CHCH₂ |
| 421. | Cl | Cl | CN | CHCH₂ |
| 422. | Br | Cl | CN | CHCH₂ |
| 423. | I | Cl | CN | CHCH₂ |
| 424. | CN | Cl | CN | CHCH₂ |
| 425. | CH₃ | Cl | CN | CHCH₂ |
| 426. | CF₃ | Cl | CN | CHCH₂ |
| 427. | OCH₃ | Cl | CN | CHCH₂ |
| 428. | OCF₃ | Cl | CN | CHCH₂ |
| 429. | CH₂CH₃ | Cl | CN | CHCH₂ |
| 430. | H | H | CH₃ | CHCH₂ |
| 431. | F | H | CH₃ | CHCH₂ |
| 432. | Cl | H | CH₃ | CHCH₂ |
| 433. | Br | H | CH₃ | CHCH₂ |
| 434. | I | H | CH₃ | CHCH₂ |
| 435. | CN | H | CH₃ | CHCH₂ |
| 436. | CH₃ | H | CH₃ | CHCH₂ |
| 437. | CF₃ | H | CH₃ | CHCH₂ |
| 438. | OCH₃ | H | CH₃ | CHCH₂ |
| 439. | OCF₃ | H | CH₃ | CHCH₂ |
| 440. | CH₂CH₃ | H | CH₃ | CHCH₂ |
| 441. | H | F | CH₃ | CHCH₂ |
| 442. | F | F | CH₃ | CHCH₂ |
| 443. | Cl | F | CH₃ | CHCH₂ |
| 444. | Br | F | CH₃ | CHCH₂ |
| 445. | I | F | CH₃ | CHCH₂ |
| 446. | CN | F | CH₃ | CHCH₂ |
| 447. | CH₃ | F | CH₃ | CHCH₂ |
| 448. | CF₃ | F | CH₃ | CHCH₂ |
| 449. | OCH₃ | F | CH₃ | CHCH₂ |
| 450. | OCF₃ | F | CH₃ | CHCH₂ |
| 451. | CH₂CH₃ | F | CH₃ | CHCH₂ |
| 452. | H | Cl | CH₃ | CHCH₂ |
| 453. | F | Cl | CH₃ | CHCH₂ |
| 454. | Cl | Cl | CH₃ | CHCH₂ |
| 455. | Br | Cl | CH₃ | CHCH₂ |
| 456. | I | Cl | CH₃ | CHCH₂ |
| 457. | CN | Cl | CH₃ | CHCH₂ |
| 458. | CH₃ | Cl | CH₃ | CHCH₂ |
| 459. | CF₃ | Cl | CH₃ | CHCH₂ |
| 460. | OCH₃ | Cl | CH₃ | CHCH₂ |
| 461. | OCF₃ | Cl | CH₃ | CHCH₂ |
| 462. | CH₂CH₃ | Cl | CH₃ | CHCH₂ |
| 463. | H | H | H | CF₃ |
| 464. | F | H | H | CF₃ |
| 465. | Cl | H | H | CF₃ |
| 466. | Br | H | H | CF₃ |
| 467. | I | H | H | CF₃ |
| 468. | CN | H | H | CF₃ |
| 469. | CH₃ | H | H | CF₃ |
| 470. | CF₃ | H | H | CF₃ |
| 471. | OCH₃ | H | H | CF₃ |
| 472. | OCF₃ | H | H | CF₃ |
| 473. | CH₂CH₃ | H | H | CF₃ |
| 474. | H | F | H | CF₃ |
| 475. | F | F | H | CF₃ |
| 476. | Cl | F | H | CF₃ |
| 477. | Br | F | H | CF₃ |
| 478. | I | F | H | CF₃ |
| 479. | CN | F | H | CF₃ |
| 480. | CH₃ | F | H | CF₃ |
| 481. | CF₃ | F | H | CF₃ |
| 482. | OCH₃ | F | H | CF₃ |
| 483. | OCF₃ | F | H | CF₃ |
| 484. | CH₂CH₃ | F | H | CF₃ |
| 485. | H | Cl | H | CF₃ |
| 486. | F | Cl | H | CF₃ |
| 487. | Cl | Cl | H | CF₃ |
| 488. | Br | Cl | H | CF₃ |
| 489. | I | Cl | H | CF₃ |
| 490. | CN | Cl | H | CF₃ |
| 491. | CH₃ | Cl | H | CF₃ |
| 492. | CF₃ | Cl | H | CF₃ |
| 493. | OCH₃ | Cl | H | CF₃ |
| 494. | OCF₃ | Cl | H | CF₃ |
| 495. | CH₂CH₃ | Cl | H | CF₃ |
| 496. | H | H | F | CF₃ |
| 497. | F | H | F | CF₃ |
| 498. | Cl | H | F | CF₃ |
| 499. | Br | H | F | CF₃ |
| 500. | I | H | F | CF₃ |
| 501. | CN | H | F | CF₃ |
| 502. | CH₃ | H | F | CF₃ |
| 503. | CF₃ | H | F | CF₃ |
| 504. | OCH₃ | H | F | CF₃ |
| 505. | OCF₃ | H | F | CF₃ |
| 506. | CH₂CH₃ | H | F | CF₃ |
| 507. | H | F | F | CF₃ |
| 508. | F | F | F | CF₃ |
| 509. | Cl | F | F | CF₃ |
| 510. | Br | F | F | CF₃ |
| 511. | I | F | F | CF₃ |
| 512. | CN | F | F | CF₃ |
| 513. | CH₃ | F | F | CF₃ |
| 514. | CF₃ | F | F | CF₃ |
| 515. | OCH₃ | F | F | CF₃ |
| 516. | OCF₃ | F | F | CF₃ |
| 517. | CH₂CH₃ | F | F | CF₃ |
| 518. | H | Cl | F | CF₃ |
| 519. | F | Cl | F | CF₃ |
| 520. | Cl | Cl | F | CF₃ |
| 521. | Br | Cl | F | CF₃ |
| 522. | I | Cl | F | CF₃ |
| 523. | CN | Cl | F | CF₃ |
| 524. | CH₃ | Cl | F | CF₃ |
| 525. | CF₃ | Cl | F | CF₃ |
| 526. | OCH₃ | Cl | F | CF₃ |
| 527. | OCF₃ | Cl | F | CF₃ |
| 528. | CH₂CH₃ | Cl | F | CF₃ |
| 529. | H | H | Cl | CF₃ |
| 530. | F | H | Cl | CF₃ |
| 531. | Cl | H | Cl | CF₃ |
| 532. | Br | H | Cl | CF₃ |
| 533. | I | H | Cl | CF₃ |
| 534. | CN | H | Cl | CF₃ |
| 535. | CH₃ | H | Cl | CF₃ |
| 536. | CF₃ | H | Cl | CF₃ |
| 537. | OCH₃ | H | Cl | CF₃ |
| 538. | OCF₃ | H | Cl | CF₃ |
| 539. | CH₂CH₃ | H | Cl | CF₃ |
| 540. | H | F | Cl | CF₃ |
| 541. | F | F | Cl | CF₃ |
| 542. | Cl | F | Cl | CF₃ |
| 543. | Br | F | Cl | CF₃ |
| 544. | I | F | Cl | CF₃ |
| 545. | CN | F | Cl | CF₃ |
| 546. | CH₃ | F | Cl | CF₃ |
| 547. | CF₃ | F | Cl | CF₃ |
| 548. | OCH₃ | F | Cl | CF₃ |
| 549. | OCF₃ | F | Cl | CF₃ |
| 550. | CH₂CH₃ | F | Cl | CF₃ |
| 551. | H | Cl | Cl | CF₃ |
| 552. | F | Cl | Cl | CF₃ |
| 553. | Cl | Cl | Cl | CF₃ |
| 554. | Br | Cl | Cl | CF₃ |
| 555. | I | Cl | Cl | CF₃ |
| 556. | CN | Cl | Cl | CF₃ |
| 557. | CH₃ | Cl | Cl | CF₃ |
| 558. | CF₃ | Cl | Cl | CF₃ |
| 559. | OCH₃ | Cl | Cl | CF₃ |
| 560. | OCF₃ | Cl | Cl | CF₃ |
| 561. | CH₂CH₃ | Cl | Cl | CF₃ |
| 562. | H | H | Br | CF₃ |
| 563. | F | H | Br | CF₃ |
| 564. | Cl | H | Br | CF₃ |
| 565. | Br | H | Br | CF₃ |
| 566. | I | H | Br | CF₃ |
| 567. | CN | H | Br | CF₃ |
| 568. | CH₃ | H | Br | CF₃ |
| 569. | CF₃ | H | Br | CF₃ |
| 570. | OCH₃ | H | Br | CF₃ |
| 571. | OCF₃ | H | Br | CF₃ |
| 572. | CH₂CH₃ | H | Br | CF₃ |
| 573. | H | F | Br | CF₃ |
| 574. | F | F | Br | CF₃ |
| 575. | Cl | F | Br | CF₃ |
| 576. | Br | F | Br | CF₃ |
| 577. | I | F | Br | CF₃ |
| 578. | CN | F | Br | CF₃ |
| 579. | CH₃ | F | Br | CF₃ |
| 580. | CF₃ | F | Br | CF₃ |
| 581. | OCH₃ | F | Br | CF₃ |
| 582. | OCF₃ | F | Br | CF₃ |
| 583. | CH₂CH₃ | F | Br | CF₃ |
| 584. | H | Cl | Br | CF₃ |
| 585. | F | Cl | Br | CF₃ |
| 586. | Cl | Cl | Br | CF₃ |
| 587. | Br | Cl | Br | CF₃ |
| 588. | I | Cl | Br | CF₃ |
| 589. | CN | Cl | Br | CF₃ |
| 590. | CH₃ | Cl | Br | CF₃ |
| 591. | CF₃ | Cl | Br | CF₃ |
| 592. | OCH₃ | Cl | Br | CF₃ |
| 593. | OCF₃ | Cl | Br | CF₃ |
| 594. | CH₂CH₃ | Cl | Br | CF₃ |
| 595. | H | H | I | CF₃ |
| 596. | F | H | I | CF₃ |
| 597. | Cl | H | I | CF₃ |
| 598. | Br | H | I | CF₃ |
| 599. | I | H | I | CF₃ |
| 600. | CN | H | I | CF₃ |
| 601. | CH₃ | H | I | CF₃ |
| 602. | CF₃ | H | I | CF₃ |
| 603. | OCH₃ | H | I | CF₃ |
| 604. | OCF₃ | H | I | CF₃ |
| 605. | CH₂CH₃ | H | I | CF₃ |
| 606. | H | F | I | CF₃ |
| 607. | F | F | I | CF₃ |
| 608. | Cl | F | I | CF₃ |
| 609. | Br | F | I | CF₃ |
| 610. | I | F | I | CF₃ |
| 611. | CN | F | I | CF₃ |
| 612. | CH₃ | F | I | CF₃ |
| 613. | CF₃ | F | I | CF₃ |
| 614. | OCH₃ | F | I | CF₃ |
| 615. | OCF₃ | F | I | CF₃ |
| 616. | CH₂CH₃ | F | I | CF₃ |
| 617. | H | Cl | I | CF₃ |
| 618. | F | Cl | I | CF₃ |
| 619. | Cl | Cl | I | CF₃ |
| 620. | Br | Cl | I | CF₃ |
| 621. | I | Cl | I | CF₃ |
| 622. | CN | Cl | I | CF₃ |
| 623. | CH₃ | Cl | I | CF₃ |
| 624. | CF₃ | Cl | I | CF₃ |
| 625. | OCH₃ | Cl | I | CF₃ |
| 626. | OCF₃ | Cl | I | CF₃ |
| 627. | CH₂CH₃ | Cl | I | CF₃ |
| 628. | H | H | CN | CF₃ |
| 629. | F | H | CN | CF₃ |
| 630. | Cl | H | CN | CF₃ |
| 631. | Br | H | CN | CF₃ |
| 632. | I | H | CN | CF₃ |
| 633. | CN | H | CN | CF₃ |
| 634. | CH₃ | H | CN | CF₃ |
| 635. | CF₃ | H | CN | CF₃ |
| 636. | OCH₃ | H | CN | CF₃ |
| 637. | OCF₃ | H | CN | CF₃ |
| 638. | CH₂CH₃ | H | CN | CF₃ |
| 639. | H | F | CN | CF₃ |
| 640. | F | F | CN | CF₃ |
| 641. | Cl | F | CN | CF₃ |
| 642. | Br | F | CN | CF₃ |
| 643. | I | F | CN | CF₃ |
| 644. | CN | F | CN | CF₃ |
| 645. | CH₃ | F | CN | CF₃ |
| 646. | CF₃ | F | CN | CF₃ |
| 647. | OCH₃ | F | CN | CF₃ |
| 648. | OCF₃ | F | CN | CF₃ |
| 649. | CH₂CH₃ | F | CN | CF₃ |
| 650. | H | Cl | CN | CF₃ |
| 651. | F | Cl | CN | CF₃ |
| 652. | Cl | Cl | CN | CF₃ |
| 653. | Br | Cl | CN | CF₃ |
| 654. | I | Cl | CN | CF₃ |
| 655. | CN | Cl | CN | CF₃ |
| 656. | CH₃ | Cl | CN | CF₃ |
| 657. | CF₃ | Cl | CN | CF₃ |
| 658. | OCH₃ | Cl | CN | CF₃ |
| 659. | OCF₃ | Cl | CN | CF₃ |
| 660. | CH₂CH₃ | Cl | CN | CF₃ |
| 661. | H | H | CH₃ | CF₃ |
| 662. | F | H | CH₃ | CF₃ |
| 663. | Cl | H | CH₃ | CF₃ |
| 664. | Br | H | CH₃ | CF₃ |
| 665. | I | H | CH₃ | CF₃ |
| 666. | CN | H | CH₃ | CF₃ |
| 667. | CH₃ | H | CH₃ | CF₃ |
| 668. | CF₃ | H | CH₃ | CF₃ |
| 669. | OCH₃ | H | CH₃ | CF₃ |
| 670. | OCF₃ | H | CH₃ | CF₃ |
| 671. | CH₂CH₃ | H | CH₃ | CF₃ |
| 672. | H | F | CH₃ | CF₃ |
| 673. | F | F | CH₃ | CF₃ |
| 674. | Cl | F | CH₃ | CF₃ |
| 675. | Br | F | CH₃ | CF₃ |
| 676. | I | F | CH₃ | CF₃ |
| 677. | CN | F | CH₃ | CF₃ |
| 678. | CH₃ | F | CH₃ | CF₃ |
| 679. | CF₃ | F | CH₃ | CF₃ |
| 680. | OCH₃ | F | CH₃ | CF₃ |
| 681. | OCF₃ | F | CH₃ | CF₃ |
| 682. | CH₂CH₃ | F | CH₃ | CF₃ |
| 683. | H | Cl | CH₃ | CF₃ |
| 684. | F | Cl | CH₃ | CF₃ |
| 685. | Cl | Cl | CH₃ | CF₃ |
| 686. | Br | Cl | CH₃ | CF₃ |
| 687. | I | Cl | CH₃ | CF₃ |
| 688. | CN | Cl | CH₃ | CF₃ |
| 689. | CH₃ | Cl | CH₃ | CF₃ |
| 690. | CF₃ | Cl | CH₃ | CF₃ |
| 691. | OCH₃ | Cl | CH₃ | CF₃ |
| 692. | OCF₃ | Cl | CH₃ | CF₃ |
| 693. | CH₂CH₃ | Cl | CH₃ | CF₃ |
| 694. | H | H | H | 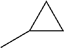 |
| 695. | F | H | H | 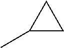 |
| 696. | Cl | H | H | 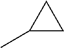 |
| 697. | Br | H | H | 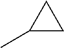 |
| 698. | I | H | H | 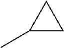 |
| 699. | CN | H | H | 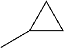 |
| 700. | CH₃ | H | H | 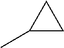 |
| 701. | CF₃ | H | H | 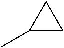 |
| 702. | OCH₃ | H | H | 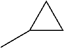 |
| 703. | OCF₃ | H | H | 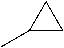 |
| 704. | CH₂CH₃ | H | H | 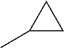 |
| 705. | H | F | H | 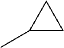 |
| 706. | F | F | H | 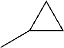 |
| 707. | Cl | F | H | 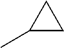 |
| 708. | Br | F | H | 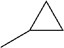 |
| 709. | I | F | H | 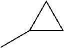 |
| 710. | CN | F | H | 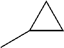 |
| 711. | CH₃ | F | H | 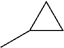 |
| 712. | CF₃ | F | H | 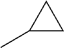 |
| 713. | OCH₃ | F | H | 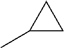 |
| 714. | OCF₃ | F | H | 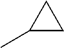 |
| 715. | CH₂CH₃ | F | H | 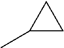 |
| 716. | H | Cl | H | 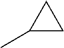 |
| 717. | F | Cl | H | 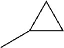 |
| 718. | Cl | Cl | H | 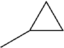 |
| 719. | Br | Cl | H | 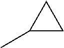 |
| 720. | I | Cl | H | 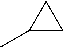 |
| 721. | CN | Cl | H | 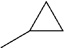 |
| 722. | CH₃ | Cl | H | 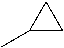 |
| 723. | CF₃ | Cl | H | 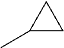 |
| 724. | OCH₃ | Cl | H | 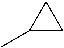 |
| 725. | OCF₃ | Cl | H | 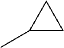 |
| 726. | CH₂CH₃ | Cl | H | 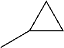 |
| 727. | H | H | F | 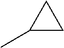 |
| 728. | F | H | F | 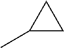 |
| 729. | Cl | H | F | 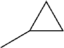 |
| 730. | Br | H | F | 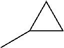 |
| 731. | I | H | F | 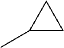 |
| 732. | CN | H | F | 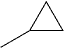 |
| 733. | CH₃ | H | F | 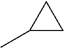 |
| 734. | CF₃ | H | F | 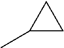 |
| 735. | OCH₃ | H | F | 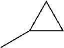 |
| 736. | OCF₃ | H | F | 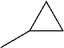 |
| 737. | CH₂CH₃ | H | F | 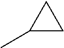 |
| 738. | H | F | F | 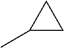 |
| 739. | F | F | F | 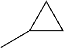 |
| 740. | Cl | F | F | 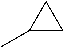 |
| 741. | Br | F | F | 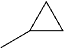 |
| 742. | I | F | F | 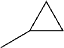 |
| 743. | CN | F | F | 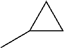 |
| 744. | CH₃ | F | F | 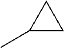 |
| 745. | CF₃ | F | F | 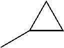 |
| 746. | OCH₃ | F | F | 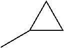 |
| 747. | OCF₃ | F | F | 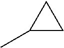 |
| 748. | CH₂CH₃ | F | F | 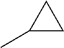 |
| 749. | H | Cl | F | 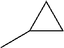 |
| 750. | F | Cl | F | 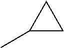 |
| 751. | Cl | Cl | F | 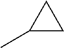 |
| 752. | Br | Cl | F | 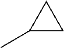 |
| 753. | I | Cl | F | 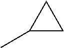 |
| 754. | CN | Cl | F | 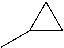 |
| 755. | CH₃ | Cl | F | 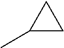 |
| 756. | CF₃ | Cl | F | 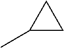 |
| 757. | OCH₃ | Cl | F | 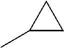 |
| 758. | OCF₃ | Cl | F | 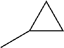 |
| 759. | CH₂CH₃ | Cl | F | 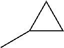 |
| 760. | H | H | Cl | 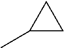 |
| 761. | F | H | Cl | 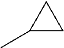 |
| 762. | Cl | H | Cl | 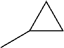 |
| 763. | Br | H | Cl | 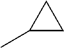 |
| 764. | I | H | Cl | 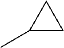 |
| 765. | CN | H | Cl | 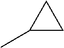 |
| 766. | CH₃ | H | Cl | 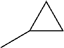 |
| 767. | CF₃ | H | Cl | 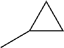 |
| 768. | OCH₃ | H | Cl | 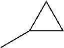 |
| 769. | OCF₃ | H | Cl | 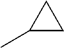 |
| 770. | CH₂CH₃ | H | Cl | 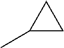 |
| 771. | H | F | Cl | 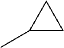 |
| 772. | F | F | Cl | 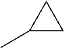 |
| 773. | Cl | F | Cl | 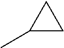 |
| 774. | Br | F | Cl | 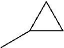 |
| 775. | I | F | Cl | 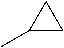 |
| 776. | CN | F | Cl | 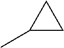 |
| 777. | CH₃ | F | Cl | 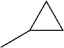 |
| 778. | CF₃ | F | Cl | 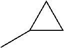 |
| 779. | OCH₃ | F | Cl | 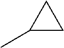 |
| 780. | OCF₃ | F | Cl | 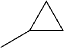 |
| 781. | CH₂CH₃ | F | Cl | 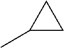 |
| 782. | H | Cl | Cl | 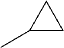 |
| 783. | F | Cl | Cl | 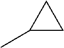 |
| 784. | Cl | Cl | Cl | 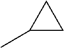 |
| 785. | Br | Cl | Cl | 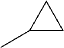 |
| 786. | I | Cl | Cl | 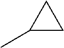 |
| 787. | CN | Cl | Cl | 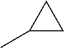 |
| 788. | CH₃ | Cl | Cl | 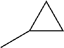 |
| 789. | CF₃ | Cl | Cl | 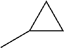 |
| 790. | OCH₃ | Cl | Cl | 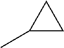 |
| 791. | OCF₃ | Cl | Cl | 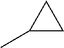 |
| 792. | CH₂CH₃ | Cl | Cl | 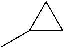 |
| 793. | H | H | Br | 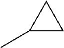 |
| 794. | F | H | Br | 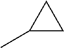 |
| 795. | Cl | H | Br | 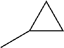 |
| 796. | Br | H | Br | 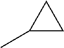 |
| 797. | I | H | Br | 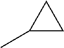 |
| 798. | CN | H | Br | 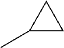 |
| 799. | CH₃ | H | Br | 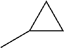 |
| 800. | CF₃ | H | Br | 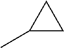 |
| 801. | OCH₃ | H | Br | 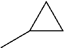 |
| 802. | OCF₃ | H | Br | 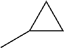 |
| 803. | CH₂CH₃ | H | Br | 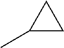 |
| 804. | H | F | Br | 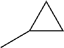 |
| 805. | F | F | Br | 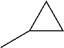 |
| 806. | Cl | F | Br | 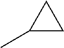 |
| 807. | Br | F | Br | 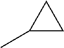 |
| 808. | I | F | Br | 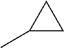 |
| 809. | CN | F | Br | 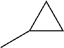 |
| 810. | CH₃ | F | Br | 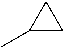 |
| 811. | CF₃ | F | Br | 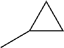 |
| 812. | OCH₃ | F | Br | 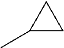 |
| 813. | OCF₃ | F | Br | 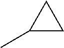 |
| 814. | CH₂CH₃ | F | Br | 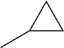 |
| 815. | H | Cl | Br | 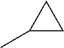 |
| 816. | F | Cl | Br | 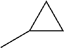 |
| 817. | Cl | Cl | Br | 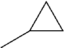 |
| 818. | Br | Cl | Br | 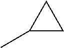 |
| 819. | I | Cl | Br | 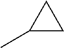 |
| 820. | CN | Cl | Br | 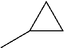 |
| 821. | CH₃ | Cl | Br | 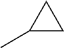 |
| 822. | CF₃ | Cl | Br | 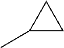 |
| 823. | OCH₃ | Cl | Br | 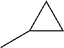 |
| 824. | OCF₃ | Cl | Br | 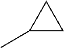 |
| 825. | CH₂CH₃ | Cl | Br | 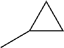 |
| 826. | H | H | I | 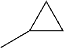 |
| 827. | F | H | I | 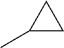 |
| 828. | Cl | H | I | 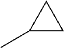 |
| 829. | Br | H | I | 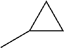 |
| 830. | I | H | I | 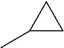 |
| 831. | CN | H | I | 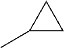 |
| 832. | CH₃ | H | I | 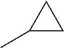 |
| 833. | CF₃ | H | I | 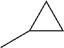 |
| 834. | OCH₃ | H | I | 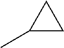 |
| 835. | OCF₃ | H | I | 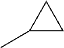 |
| 836. | CH₂CH₃ | H | I | 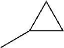 |
| 837. | H | F | I | 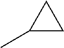 |
| 838. | F | F | I | 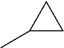 |
| 839. | Cl | F | I | 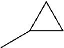 |
| 840. | Br | F | I | 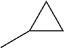 |
| 841. | I | F | I | 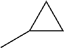 |
| 842. | CN | F | I | 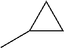 |
| 843. | CH₃ | F | I | 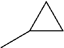 |
| 844. | CF₃ | F | I | 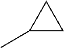 |
| 845. | OCH₃ | F | I | 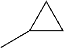 |
| 846. | OCF₃ | F | I | 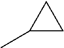 |
| 847. | CH₂CH₃ | F | I | 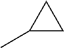 |
| 848. | H | Cl | I | 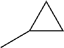 |
| 849. | F | Cl | I | 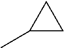 |
| 850. | Cl | Cl | I | 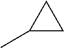 |
| 851. | Br | Cl | I | 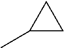 |
| 852. | I | Cl | I | 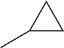 |
| 853. | CN | Cl | I | 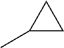 |
| 854. | CH₃ | Cl | I | 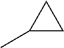 |
| 855. | CF₃ | Cl | I | 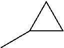 |
| 856. | OCH₃ | Cl | I | 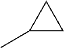 |
| 857. | OCF₃ | Cl | I | 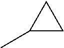 |
| 858. | CH₂CH₃ | Cl | I | 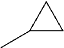 |
| 859. | H | H | CN | 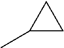 |
| 860. | F | H | CN | 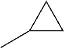 |
| 861. | Cl | H | CN | 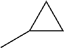 |
| 862. | Br | H | CN | 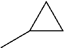 |
| 863. | I | H | CN | 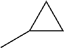 |
| 864. | CN | H | CN | 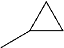 |
| 865. | CH₃ | H | CN | 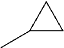 |
| 866. | CF₃ | H | CN | 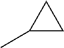 |
| 867. | OCH₃ | H | CN | 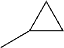 |
| 868. | OCF₃ | H | CN | 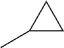 |
| 869. | CH₂CH₃ | H | CN | 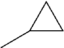 |
| 870. | H | F | CN | 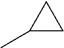 |
| 871. | F | F | CN | 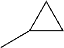 |
| 872. | Cl | F | CN | 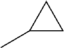 |
| 873. | Br | F | CN | 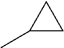 |
| 874. | I | F | CN | 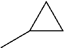 |
| 875. | CN | F | CN | 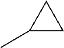 |
| 876. | CH₃ | F | CN | 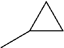 |
| 877. | CF₃ | F | CN | 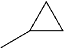 |
| 878. | OCH₃ | F | CN | 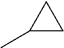 |
| 879. | OCF₃ | F | CN | 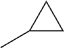 |
| 880. | CH₂CH₃ | F | CN | 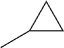 |
| 881. | H | Cl | CN | 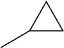 |
| 882. | F | Cl | CN | 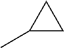 |
| 883. | Cl | Cl | CN | 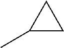 |
| 884. | Br | Cl | CN | 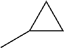 |
| 885. | I | Cl | CN | 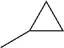 |
| 886. | CN | Cl | CN | 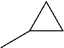 |
| 887. | CH₃ | Cl | CN | 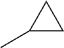 |
| 888. | CF₃ | Cl | CN | 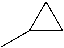 |
| 889. | OCH₃ | Cl | CN | 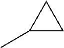 |
| 890. | OCF₃ | Cl | CN | 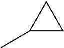 |
| 891. | CH₂CH₃ | Cl | CN | 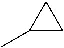 |
| 892. | H | H | CH₃ | 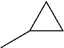 |
| 893. | F | H | CH₃ | 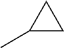 |
| 894. | Cl | H | CH₃ | 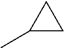 |
| 895. | Br | H | CH₃ | 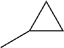 |
| 896. | I | H | CH₃ | 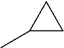 |
| 897. | CN | H | CH₃ | 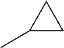 |
| 898. | CH₃ | H | CH₃ | 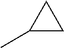 |
| 899. | CF₃ | H | CH₃ | 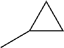 |
| 900. | OCH₃ | H | CH₃ | 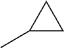 |
| 901. | OCF₃ | H | CH₃ | 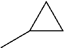 |
| 902. | CH₂CH₃ | H | CH₃ | 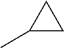 |
| 903. | H | F | CH₃ | 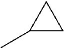 |
| 904. | F | F | CH₃ | 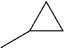 |
| 905. | Cl | F | CH₃ | 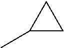 |
| 906. | Br | F | CH₃ | 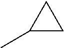 |
| 907. | I | F | CH₃ | 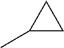 |
| 908. | CN | F | CH₃ | 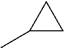 |
| 909. | CH₃ | F | CH₃ | 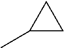 |
| 910. | CF₃ | F | CH₃ | 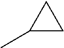 |
| 911. | OCH₃ | F | CH₃ | 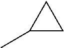 |
| 912. | OCF₃ | F | CH₃ | 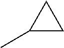 |
| 913. | CH₂CH₃ | F | CH₃ | 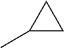 |
| 914. | H | Cl | CH₃ | 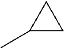 |
| 915. | F | Cl | CH₃ | 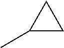 |
| 916. | Cl | Cl | CH₃ | 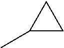 |
| 917. | Br | Cl | CH₃ | 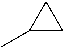 |
| 918. | I | Cl | CH₃ | 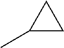 |
| 919. | CN | Cl | CH₃ | 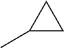 |
| 920. | CH₃ | Cl | CH₃ | 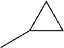 |
| 921. | CF₃ | Cl | CH₃ | 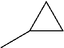 |
| 922. | OCH₃ | Cl | CH₃ | 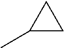 |
| 923. | OCF₃ | Cl | CH₃ | 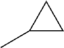 |
| 924. | CH₂CH₃ | Cl | CH₃ | 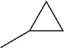 |
| 925. | H | H | H | CH₂CH₃ |
| 926. | F | H | H | CH₂CH₃ |
| 927. | Cl | H | H | CH₂CH₃ |
| 928. | Br | H | H | CH₂CH₃ |
| 929. | I | H | H | CH₂CH₃ |
| 930. | CN | H | H | CH₂CH₃ |
| 931. | CH₃ | H | H | CH₂CH₃ |
| 932. | CF₃ | H | H | CH₂CH₃ |
| 933. | OCH₃ | H | H | CH₂CH₃ |
| 934. | OCF₃ | H | H | CH₂CH₃ |
| 935. | CH₂CH₃ | H | H | CH₂CH₃ |
| 936. | H | F | H | CH₂CH₃ |
| 937. | F | F | H | CH₂CH₃ |
| 938. | Cl | F | H | CH₂CH₃ |
| 939. | Br | F | H | CH₂CH₃ |
| 940. | I | F | H | CH₂CH₃ |
| 941. | CN | F | H | CH₂CH₃ |
| 942. | CH₃ | F | H | CH₂CH₃ |
| 943. | CF₃ | F | H | CH₂CH₃ |
| 944. | OCH₃ | F | H | CH₂CH₃ |
| 945. | OCF₃ | F | H | CH₂CH₃ |
| 946. | CH₂CH₃ | F | H | CH₂CH₃ |
| 947. | H | Cl | H | CH₂CH₃ |
| 948. | F | Cl | H | CH₂CH₃ |
| 949. | Cl | Cl | H | CH₂CH₃ |
| 950. | Br | Cl | H | CH₂CH₃ |
| 951. | I | Cl | H | CH₂CH₃ |
| 952. | CN | Cl | H | CH₂CH₃ |
| 953. | CH₃ | Cl | H | CH₂CH₃ |
| 954. | CF₃ | Cl | H | CH₂CH₃ |
| 955. | OCH₃ | Cl | H | CH₂CH₃ |
| 956. | OCF₃ | Cl | H | CH₂CH₃ |
| 957. | CH₂CH₃ | Cl | H | CH₂CH₃ |
| 958. | H | H | F | CH₂CH₃ |
| 959. | F | H | F | CH₂CH₃ |
| 960. | Cl | H | F | CH₂CH₃ |
| 961. | Br | H | F | CH₂CH₃ |
| 962. | I | H | F | CH₂CH₃ |
| 963. | CN | H | F | CH₂CH₃ |
| 964. | CH₃ | H | F | CH₂CH₃ |
| 965. | CF₃ | H | F | CH₂CH₃ |
| 966. | OCH₃ | H | F | CH₂CH₃ |
| 967. | OCF₃ | H | F | CH₂CH₃ |
| 968. | CH₂CH₃ | H | F | CH₂CH₃ |
| 969. | H | F | F | CH₂CH₃ |
| 970. | F | F | F | CH₂CH₃ |
| 971. | Cl | F | F | CH₂CH₃ |
| 972. | Br | F | F | CH₂CH₃ |
| 973. | I | F | F | CH₂CH₃ |
| 974. | CN | F | F | CH₂CH₃ |
| 975. | CH₃ | F | F | CH₂CH₃ |
| 976. | CF₃ | F | F | CH₂CH₃ |
| 977. | OCH₃ | F | F | CH₂CH₃ |
| 978. | OCF₃ | F | F | CH₂CH₃ |
| 979. | CH₂CH₃ | F | F | CH₂CH₃ |
| 980. | H | Cl | F | CH₂CH₃ |
| 981. | F | Cl | F | CH₂CH₃ |
| 982. | Cl | Cl | F | CH₂CH₃ |
| 983. | Br | Cl | F | CH₂CH₃ |
| 984. | I | Cl | F | CH₂CH₃ |
| 985. | CN | Cl | F | CH₂CH₃ |
| 986. | CH₃ | Cl | F | CH₂CH₃ |
| 987. | CF₃ | Cl | F | CH₂CH₃ |
| 988. | OCH₃ | Cl | F | CH₂CH₃ |
| 989. | OCF₃ | Cl | F | CH₂CH₃ |
| 990. | CH₂CH₃ | Cl | F | CH₂CH₃ |
| 991. | H | H | Cl | CH₂CH₃ |
| 992. | F | H | Cl | CH₂CH₃ |
| 993. | Cl | H | Cl | CH₂CH₃ |
| 994. | Br | H | Cl | CH₂CH₃ |
| 995. | I | H | Cl | CH₂CH₃ |
| 996. | CN | H | Cl | CH₂CH₃ |
| 997. | CH₃ | H | Cl | CH₂CH₃ |
| 998. | CF₃ | H | Cl | CH₂CH₃ |
| 999. | OCH₃ | H | Cl | CH₂CH₃ |
| 1000. | OCF₃ | H | Cl | CH₂CH₃ |
| 1001. | CH₂CH₃ | H | Cl | CH₂CH₃ |
| 1002. | H | F | Cl | CH₂CH₃ |
| 1003. | F | F | Cl | CH₂CH₃ |
| 1004. | Cl | F | Cl | CH₂CH₃ |
| 1005. | Br | F | Cl | CH₂CH₃ |
| 1006. | I | F | Cl | CH₂CH₃ |
| 1007. | CN | F | Cl | CH₂CH₃ |
| 1008. | CH₃ | F | Cl | CH₂CH₃ |
| 1009. | CF₃ | F | Cl | CH₂CH₃ |
| 1010. | OCH₃ | F | Cl | CH₂CH₃ |
| 1011. | OCF₃ | F | Cl | CH₂CH₃ |
| 1012. | CH₂CH₃ | F | Cl | CH₂CH₃ |
| 1013. | H | Cl | Cl | CH₂CH₃ |
| 1014. | F | Cl | Cl | CH₂CH₃ |
| 1015. | Cl | Cl | Cl | CH₂CH₃ |
| 1016. | Br | Cl | Cl | CH₂CH₃ |
| 1017. | I | Cl | Cl | CH₂CH₃ |
| 1018. | CN | Cl | Cl | CH₂CH₃ |
| 1019. | CH₃ | Cl | Cl | CH₂CH₃ |
| 1020. | CF₃ | Cl | Cl | CH₂CH₃ |
| 1021. | OCH₃ | Cl | Cl | CH₂CH₃ |
| 1022. | OCF₃ | Cl | Cl | CH₂CH₃ |
| 1023. | CH₂CH₃ | Cl | Cl | CH₂CH₃ |
| 1024. | H | H | Br | CH₂CH₃ |
| 1025. | F | H | Br | CH₂CH₃ |
| 1026. | Cl | H | Br | CH₂CH₃ |
| 1027. | Br | H | Br | CH₂CH₃ |
| 1028. | I | H | Br | CH₂CH₃ |
| 1029. | CN | H | Br | CH₂CH₃ |
| 1030. | CH₃ | H | Br | CH₂CH₃ |
| 1031. | CF₃ | H | Br | CH₂CH₃ |
| 1032. | OCH₃ | H | Br | CH₂CH₃ |
| 1033. | OCF₃ | H | Br | CH₂CH₃ |
| 1034. | CH₂CH₃ | H | Br | CH₂CH₃ |
| 1035. | H | F | Br | CH₂CH₃ |
| 1036. | F | F | Br | CH₂CH₃ |
| 1037. | Cl | F | Br | CH₂CH₃ |
| 1038. | Br | F | Br | CH₂CH₃ |
| 1039. | I | F | Br | CH₂CH₃ |
| 1040. | CN | F | Br | CH₂CH₃ |
| 1041. | CH₃ | F | Br | CH₂CH₃ |
| 1042. | CF₃ | F | Br | CH₂CH₃ |
| 1043. | OCH₃ | F | Br | CH₂CH₃ |
| 1044. | OCF₃ | F | Br | CH₂CH₃ |
| 1045. | CH₂CH₃ | F | Br | CH₂CH₃ |
| 1046. | H | Cl | Br | CH₂CH₃ |
| 1047. | F | Cl | Br | CH₂CH₃ |
| 1048. | Cl | Cl | Br | CH₂CH₃ |
| 1049. | Br | Cl | Br | CH₂CH₃ |
| 1050. | I | Cl | Br | CH₂CH₃ |
| 1051. | CN | Cl | Br | CH₂CH₃ |
| 1052. | CH₃ | Cl | Br | CH₂CH₃ |
| 1053. | CF₃ | Cl | Br | CH₂CH₃ |
| 1054. | OCH₃ | Cl | Br | CH₂CH₃ |
| 1055. | OCF₃ | Cl | Br | CH₂CH₃ |
| 1056. | CH₂CH₃ | Cl | Br | CH₂CH₃ |
| 1057. | H | H | I | CH₂CH₃ |
| 1058. | F | H | I | CH₂CH₃ |
| 1059. | Cl | H | I | CH₂CH₃ |
| 1060. | Br | H | I | CH₂CH₃ |
| 1061. | I | H | I | CH₂CH₃ |
| 1062. | CN | H | I | CH₂CH₃ |
| 1063. | CH₃ | H | I | CH₂CH₃ |
| 1064. | CF₃ | H | I | CH₂CH₃ |
| 1065. | OCH₃ | H | I | CH₂CH₃ |
| 1066. | OCF₃ | H | I | CH₂CH₃ |
| 1067. | CH₂CH₃ | H | I | CH₂CH₃ |
| 1068. | H | F | I | CH₂CH₃ |
| 1069. | F | F | I | CH₂CH₃ |
| 1070. | Cl | F | I | CH₂CH₃ |
| 1071. | Br | F | I | CH₂CH₃ |
| 1072. | I | F | I | CH₂CH₃ |
| 1073. | CN | F | I | CH₂CH₃ |
| 1074. | CH₃ | F | I | CH₂CH₃ |
| 1075. | CF₃ | F | I | CH₂CH₃ |
| 1076. | OCH₃ | F | I | CH₂CH₃ |
| 1077. | OCF₃ | F | I | CH₂CH₃ |
| 1078. | CH₂CH₃ | F | I | CH₂CH₃ |
| 1079. | H | Cl | I | CH₂CH₃ |
| 1080. | F | Cl | I | CH₂CH₃ |
| 1081. | Cl | Cl | I | CH₂CH₃ |
| 1082. | Br | Cl | I | CH₂CH₃ |
| 1083. | I | Cl | I | CH₂CH₃ |
| 1084. | CN | Cl | I | CH₂CH₃ |
| 1085. | CH₃ | Cl | I | CH₂CH₃ |
| 1086. | CF₃ | Cl | I | CH₂CH₃ |
| 1087. | OCH₃ | Cl | I | CH₂CH₃ |
| 1088. | OCF₃ | Cl | I | CH₂CH₃ |
| 1089. | CH₂CH₃ | Cl | I | CH₂CH₃ |
| 1090. | H | H | CN | CH₂CH₃ |
| 1091. | F | H | CN | CH₂CH₃ |
| 1092. | Cl | H | CN | CH₂CH₃ |
| 1093. | Br | H | CN | CH₂CH₃ |
| 1094. | I | H | CN | CH₂CH₃ |
| 1095. | CN | H | CN | CH₂CH₃ |
| 1096. | CH₃ | H | CN | CH₂CH₃ |
| 1097. | CF₃ | H | CN | CH₂CH₃ |
| 1098. | OCH₃ | H | CN | CH₂CH₃ |
| 1099. | OCF₃ | H | CN | CH₂CH₃ |
| 1100. | CH₂CH₃ | H | CN | CH₂CH₃ |
| 1101. | H | F | CN | CH₂CH₃ |
| 1102. | F | F | CN | CH₂CH₃ |
| 1103. | Cl | F | CN | CH₂CH₃ |
| 1104. | Br | F | CN | CH₂CH₃ |
| 1105. | I | F | CN | CH₂CH₃ |
| 1106. | CN | F | CN | CH₂CH₃ |
| 1107. | CH₃ | F | CN | CH₂CH₃ |
| 1108. | CF₃ | F | CN | CH₂CH₃ |
| 1109. | OCH₃ | F | CN | CH₂CH₃ |
| 1110. | OCF₃ | F | CN | CH₂CH₃ |
| 1111. | CH₂CH₃ | F | CN | CH₂CH₃ |
| 1112. | H | Cl | CN | CH₂CH₃ |
| 1113. | F | Cl | CN | CH₂CH₃ |
| 1114. | Cl | Cl | CN | CH₂CH₃ |
| 1115. | Br | Cl | CN | CH₂CH₃ |
| 1116. | I | Cl | CN | CH₂CH₃ |
| 1117. | CN | Cl | CN | CH₂CH₃ |
| 1118. | CH₃ | Cl | CN | CH₂CH₃ |
| 1119. | CF₃ | Cl | CN | CH₂CH₃ |
| 1120. | OCH₃ | Cl | CN | CH₂CH₃ |
| 1121. | OCF₃ | Cl | CN | CH₂CH₃ |
| 1122. | CH₂CH₃ | Cl | CN | CH₂CH₃ |
| 1123. | H | H | CH₃ | CH₂CH₃ |
| 1124. | F | H | CH₃ | CH₂CH₃ |
| 1125. | Cl | H | CH₃ | CH₂CH₃ |
| 1126. | Br | H | CH₃ | CH₂CH₃ |
| 1127. | I | H | CH₃ | CH₂CH₃ |
| 1128. | CN | H | CH₃ | CH₂CH₃ |
| 1129. | CH₃ | H | CH₃ | CH₂CH₃ |
| 1130. | CF₃ | H | CH₃ | CH₂CH₃ |
| 1131. | OCH₃ | H | CH₃ | CH₂CH₃ |
| 1132. | OCF₃ | H | CH₃ | CH₂CH₃ |
| 1133. | CH₂CH₃ | H | CH₃ | CH₂CH₃ |
| 1134. | H | F | CH₃ | CH₂CH₃ |
| 1135. | F | F | CH₃ | CH₂CH₃ |
| 1136. | Cl | F | CH₃ | CH₂CH₃ |
| 1137. | Br | F | CH₃ | CH₂CH₃ |
| 1138. | I | F | CH₃ | CH₂CH₃ |
| 1139. | CN | F | CH₃ | CH₂CH₃ |
| 1140. | CH₃ | F | CH₃ | CH₂CH₃ |
| 1141. | CF₃ | F | CH₃ | CH₂CH₃ |
| 1142. | OCH₃ | F | CH₃ | CH₂CH₃ |
| 1143. | OCF₃ | F | CH₃ | CH₂CH₃ |
| 1144. | CH₂CH₃ | F | CH₃ | CH₂CH₃ |
| 1145. | H | Cl | CH₃ | CH₂CH₃ |
| 1146. | F | Cl | CH₃ | CH₂CH₃ |
| 1147. | Cl | Cl | CH₃ | CH₂CH₃ |
| 1148. | Br | Cl | CH₃ | CH₂CH₃ |
| 1149. | I | Cl | CH₃ | CH₂CH₃ |
| 1150. | CN | Cl | CH₃ | CH₂CH₃ |
| 1151. | CH₃ | Cl | CH₃ | CH₂CH₃ |
| 1152. | CF₃ | Cl | CH₃ | CH₂CH₃ |
| 1153. | OCH₃ | Cl | CH₃ | CH₂CH₃ |
| 1154. | OCF₃ | Cl | CH₃ | CH₂CH₃ |
| 1155. | CH₂CH₃ | Cl | CH₃ | CH₂CH₃ |

Compounds of formula III.1., wherein R², R⁶, R⁸, R⁹ and R¹⁰ are hydrogen, and R³, R⁴, R⁵ and R⁷ have the meanings as defined lines in 1 to 1155 of Table 1 above, i.e. individual compounds III.1.1-III.1.1155, are particularly preferred:

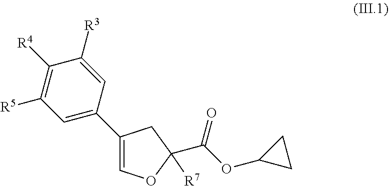

Compounds of formula III.2., wherein R², R⁶, R⁸, R⁹ and R¹⁰ are hydrogen, and R³, R⁴, R⁵ and R⁷ have the meanings as defined lines in 1 to 1155 of Table 1 above, i.e. individual compounds III.2.1-III.2. 1155, are particularly preferred:

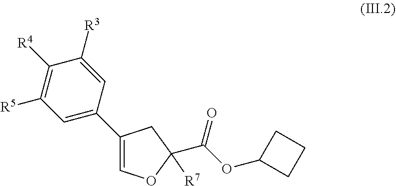

Compounds of formula III.3., wherein R², R⁶, R⁸, R⁹ and R¹⁰ are hydrogen, and R³, R⁴, R⁵ and R⁷ have the meanings as defined lines in 1 to 1155 of Table 1 above, i.e. individual compounds III.3.1-III.3.1155, are particularly preferred:

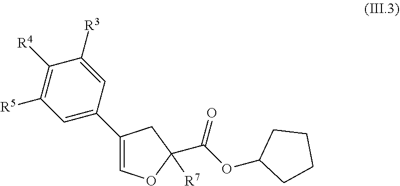

Compounds of formula III.4., wherein R², R⁶, R⁸, R⁹ and R¹⁰ are hydrogen, and R³, R⁴, R⁵ and R⁷ have the meanings as defined lines in 1 to 1155 of Table 1 above, i.e. individual compounds III.4.1-III.4.1155, are particularly preferred:

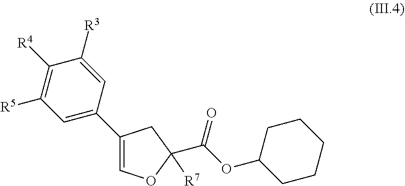

Compounds of formula III.5., wherein R², R⁶, R⁸, R⁹ and R¹⁰ are hydrogen, and R³, R⁴, R⁵ and R⁷ have the meanings as defined lines in 1 to 1155 of Table 1 above, i.e. individual compounds III.5.1-III.5.1155, are particularly preferred:

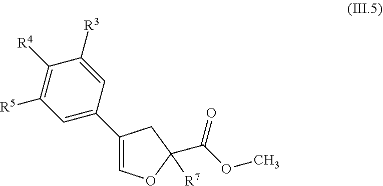

Compounds of formula III.6., wherein R², R⁶, R⁸, R⁹ and R¹⁰ are hydrogen, and R³, R⁴, R⁵ and R⁷ have the meanings as defined lines in 1 to 1155 of Table 1 above, i.e. individual compounds III.6.1-III.6.1155, are particularly preferred:

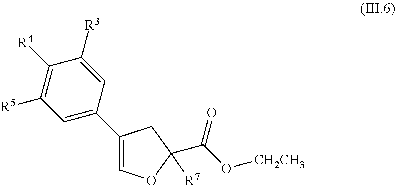

Compounds of formula IV.1., wherein R², R⁶, R⁸, R⁹ and R¹⁰ are hydrogen, and R³, R⁴, R⁵ and R⁷ have the meanings as defined lines in 1 to 1155 of Table 1 above, i.e. individual compounds IV.1.1-IV.1.1155, are particularly preferred:

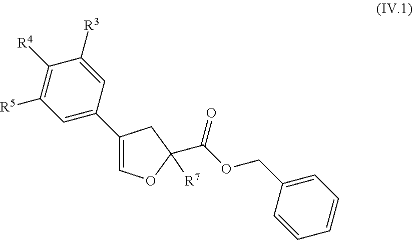

Compounds of formula IV.2., wherein R², R⁶, R⁸, R⁹ and R¹⁰ are hydrogen, and R³, R⁴, R⁵ and R⁷ have the meanings as defined lines in 1 to 1155 of Table 1 above, i.e. individual compounds IV.2.1-IV.2.1155, are particularly preferred:

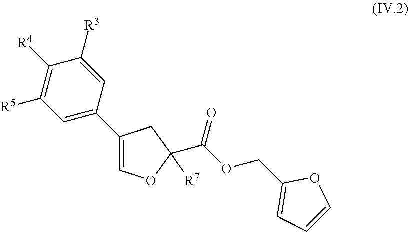

Compounds of formula IV.3., wherein R², R⁶, R⁸, R⁹ and R¹⁰ are hydrogen, and R³, R⁴, R⁵ and R⁷ have the meanings as defined lines in 1 to 1155 of Table 1 above, i.e. individual compounds IV.3.1-IV.3.1155, are particularly preferred:

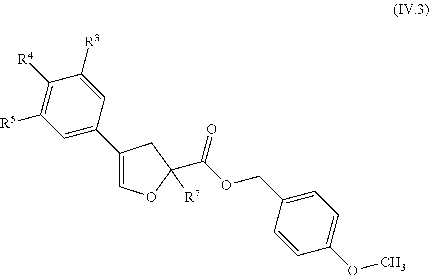

Compounds of formula IV.4., wherein R², R⁶, R⁸, R⁹ and R¹⁰ are hydrogen, and R³, R⁴, R⁵ and R⁷ have the meanings as defined lines in 1 to 1155 of Table 1 above, i.e. individual compounds IV.4.1-IV.4.1155, are particularly preferred:

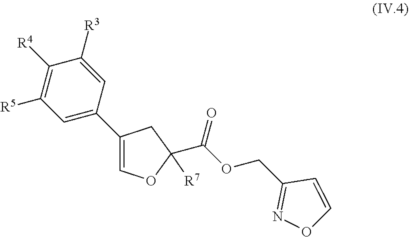

Compounds of formula IV.5., wherein R², R⁶, R⁸, R⁹ and R¹⁰ are hydrogen, and R³, R⁴, R⁵ and R⁷ have the meanings as defined lines in 1 to 1155 of Table 1 above, i.e. individual compounds IV.5.1-IV.5.1155, are particularly preferred:

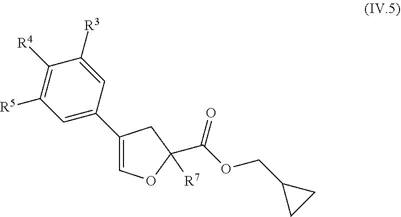

The compounds of formula (I) according to the invention can be prepared by standard processes of organic chemistry, for example by the following processes:

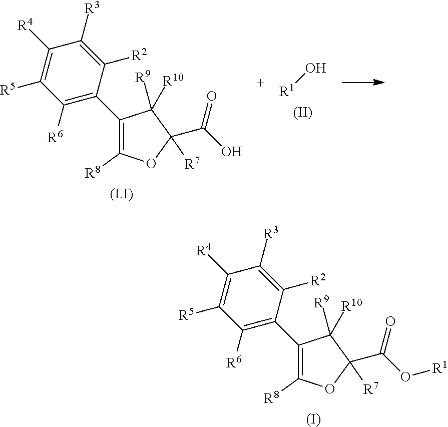

The compounds of formula (I) can be prepared according to methods or in analogy to methods that are described in the prior art. The synthesis takes advantage of starting materials that are commercially available or may be prepared according to conventional procedures starting from readily available compounds.

Compounds of the formula (I) can be prepared from the carboxylic acids (1:1) and commercially available alcohols (II) using an organic base and a coupling reagent. Thus, compounds of formula (I) can be synthesized from the corresponding carboxylic acids (1 eq.) using a coupling reagent (1-2 eq.), for example N,N′-Dicyclohexylcarbodiimide (DCC, CAS: 538-75-0) or 1-Ethyl-3-(3-dimethylaminopropyl)carbodiimide (EDC, CAS: 1892-57-5), an organic base (1-2 eq.) and the alcohol (II) (1-3 eq.). The reaction is typically carried out in an organic solvent. Preferably an aprotic organic solvent is used. Most dichloromethane (DCM) or dichloroethane (DCE) are used. The reaction is carried out at temperatures between 0° C. and reflux. Preferably the reaction is carried out at room temperature. Preferably the organic base is 4-dimethylaminopyridine (DMAP, CAS: 887925-31-7).

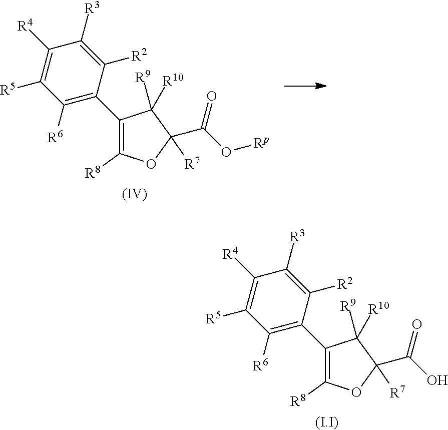

The carboxylic acids (I.I) can be prepared from the corresponding esters (IV) (wherein RP is alkyl or benzyl). If RP is alkyl, esters (IV) may be cleaved using aqueous alkali metal hydroxides. Preferably lithium hydroxide, sodium hydroxide or potassium hydroxide (1-2 eq.) are employed. The reaction is typically carried out in mixtures of water and an organic solvent. Preferably the organic solvent is THF, methanol or acetonitrile. The reaction is carried out at temperatures between 0° C. and 100° C. Preferably the reaction is carried at room temperature. If RP is benzyl in (IV), then the ester may be cleaved using palladium on charcoal (0.001-1 eq.) as catalyst and hydrogen gas at temperatures between 0° C. and reflux. Preferably the reaction is carried out at room temperature. Typically, an organic solvent is employed. Preferably THF, methanol or ethanol are employed.

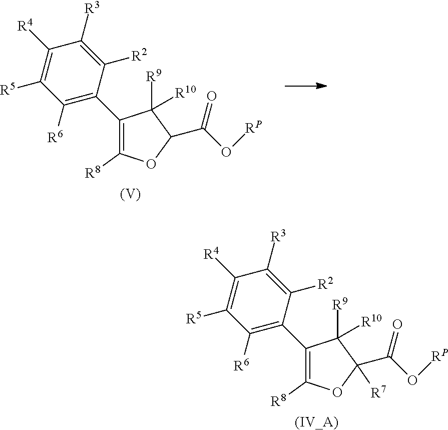

The aryldihydrofurane (IV_A) can be prepared from the corresponding unsubstituted aryldihydrofurane (V), which can be prepared according to the literature procedure J. Org. Chem. 1973, 38, 2319-2328, by deprotonation with an appropriate base and employing a commercially available electrophile. Preferably alkali amides or alkali hydrides (1-4 eq.) are used as a base. In particular, lithium bis(trimethylsilyl)amide or lithium diisopropylamide (3 eq.) are employed. As the corresponding electrophile preferably alkyl halides (4-6 eq.) are employed. The reaction is typically carried out in an aprotic organic solvent. Preferably the organic solvent is THF or di-ethyl ether. The reaction is carried out at temperatures between −78° C. and room temperature. Preferably the reaction is carried at 0° C.

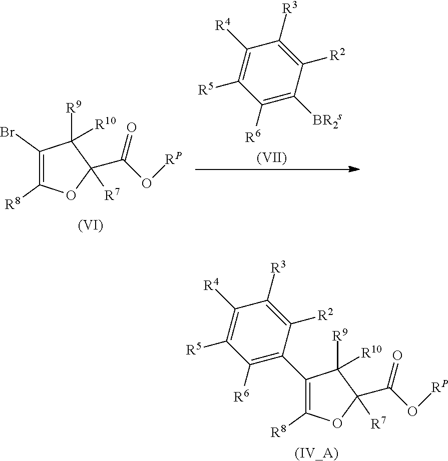

Alternatively, the aryldihydrofurane (IV_A) can be prepared from the corresponding alkenyl hal-ide by palladium-catalyzed cross coupling reaction with a commercially available organometallic compound. Preferably alkenyl bromide of the formular (VI) is employed. Preferably commercially available arylboronic acids (R⁵ is hydroxyl), aryl boronic esters (R⁵ is alkoxy), potassium tri-fluoroborates (R⁵ is fluor and potassium fluoride adduct) or arylboranes (R^{S} is alkyl) of the formular (VII) are employed in a Suzuki cross coupling. In particular, aryl boronic acid (R^{S}₂ is hydroxyl) or aryl boronic acid pinacol ester (R^{S}₂ is pinacol) are used. The reaction is typically carried out with catalytic amounts of a palladium(II) salt. Preferably [1,1′-bis(diphe-nylphosphino)ferrocene]dichloropalladium(II) (CAS: 72287-26-4) is used in equivalents ranging from 1 to 10 mol %. The reaction is typically carried out in the presence of an inorganic base. Preferably, alkali or earth alkali hydroxides or carbonates are used. In particular, sodium hydroxide or cesium carbonate are employed. The reaction is typically carried out in mixtures of water and an organic solvent. Preferably the organic solvent is THF, toluene or benzene. The reaction is carried out at elevated temperatures between room temperature and 110° C. Preferably the reaction is carried out under refluxing conditions.

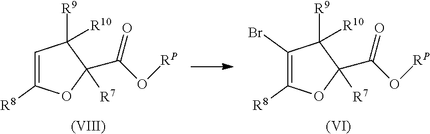

The alkenyl bromide with the formular VI can be prepared from the corresponding dihydrofu-rane, which can be prepared according to the literature procedure Tetrahedron 2003, 59, 1389-1394, by bromination followed by elimination with a suitable base. Preferably, commercially available bromination reagents are employed. In particular, bromine (CAS: 7726-95-6) is used. Preferably, commercially available organic bases are employed. In particular, non-nucleophilic bases such as diazabicyclic compounds are used. In particular, 1,8-diazabicyclo[5.4.0]undec-7-ene (DBU, CAS: 6674-22-2) or 1,5-diazabicyclo[4.3.0]non-5-ene (DBN, CAS: 3001-72-7) are employed. The reaction is typically carried out in non-protic organic solvents. Preferably, the organic solvent is halogenated. In particular, dichloromethane is employed as the solvent. The reaction is carried out under cryogenic conditions between −100 and 0° C. Preferably the reaction is carried out at −78° C.

To widen the spectrum of action, the compounds of formula (I) may be mixed with many representatives of other herbicidal or growth-regulating active ingredient groups and then applied concomitantly. Suitable components for combinations are, for example, herbicides from the classes of the acetamides, amides, aryloxyphenoxypropionates, benzamides, benzofuran, benzoic acids, benzothiadiazinones, bipyridylium, carbamates, chloroacetamides, chlorocarboxylic acids, cyclohexanediones, dinitroanilines, dinitrophenol, diphenyl ether, glycines, imidazolinones, isoxazoles, isoxazolidinones, nitriles, N-phenylphthalimides, oxadiazoles, oxazolidinediones, oxyacetamides, phenoxycarboxylic acids, phenylcarbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl(thio)benzoates, quinolinecarboxylic acids, semicarbazones, sulfonylaminocarbonyltriazolinones, sulfonylureas, tetrazolinones, thiadiazoles, thiocarbamates, triazines, triazinones, triazoles, triazolinones, triazolocarboxamides, triazolopyrimidines, triketones, uracils, ureas.

It may furthermore be beneficial to apply the compounds of formula (I) alone or in combination with other herbicides, or else in the form of a mixture with other crop protection agents, for example together with agents for controlling pests or phytopathogenic fungi or bacteria. Also of interest is the miscibility with mineral salt solutions, which are employed for treating nutritional and trace element deficiencies. Other additives such as non-phytotoxic oils and oil concentrates may also be added.

In one embodiment of the present invention the combinations according to the present invention comprise at least one compound of formula (I) (compound A or component A) and at least one further active compound selected from herbicides B (compound B), preferably herbicides B of class b1) to b15), and safeners C (compound C).

In another embodiment of the present invention the combinations according to the present invention comprise at least one compound of formula (I) and at least one further active compound B (herbicide B).

Examples of herbicides B which can be used in combination with the compounds A of formula (I) according to the present invention are:
b1) from the group of the lipid biosynthesis inhibitors:
ACC-herbicides such as alloxydim, alloxydim-sodium, butroxydim, clethodim, clodinafop, clodinafop-propargyl, cycloxydim, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, pi-noxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetra-methyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetra-methyl-2H-pyran-3-one (CAS 1312337-48-6); 5-(Acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2′,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetrame-thyl-5-oxo-2H-pyran-3-yl carbonic acid methyl ester (CAS 1033760-58-5); and non ACC herbicides such as benfuresate, butylate, cycloate, dalapon, dimepiperate, EPTC, esprocarb, ethofumesate, flupropanate, molinate, orbencarb, pebulate, prosulfocarb, TCA, thiobencarb, tio-carbazil, triallate and vernolate;
b2) from the group of the ALS inhibitors:
sulfonylureas such as amidosulfuron, azimsulfuron, bensulfuron, bensulfuron-methyl, chlorimuron, chlorimuron-ethyl, chlorsulfuron, cinosulfuron, cyclosulfamuron, ethametsulfuron, ethametsulfuron-methyl, ethoxysulfuron, flazasulfuron, flucetosulfuron, flupyrsulfuron, flupyrsul-furon-methyl-sodium, foramsulfuron, halosulfuron, halosulfuron-methyl, imazosulfuron, iodosulfuron, iodosulfuron-methyl-sodium, iofensulfuron, iofensulfuron-sodium, mesosulfuron, met-azosulfuron, metsulfuron, metsulfuron-methyl, nicosulfuron, orthosulfamuron, oxasulfuron, primisulfuron, primisulfuron-methyl, propyrisulfuron, prosulfuron, pyrazosulfuron, pyrazosulfu-ron-ethyl, rimsulfuron, sulfometuron, sulfometuron-methyl, sulfosulfuron, thifensulfuron, thifen-sulfuron-methyl, triasulfuron, tribenuron, tribenuron-methyl, trifloxysulfuron, triflusulfuron, tri-flusulfuron-methyl and tritosulfuron,
imidazolinones such as imazamethabenz, imazamethabenz-methyl, imazamox, imazapic, ima-zapyr, imazaquin and imazethapyr, triazolopyrimidine herbicides and sulfonanilides such as cloransulam, cloransulam-methyl, diclosulam, flumetsulam, florasulam, metosulam, penoxsu-lam, pyrimisulfan and pyroxsulam,
pyrimidinylbenzoates such as bispyribac, bispyribac-sodium, pyribenzoxim, pyriftalid, pyriminobac, pyriminobac-methyl, pyrithiobac, pyrithiobac-sodium, 4-[[[2-[(4,6-dimethoxy-2-pyrimidinyl)oxy]phenyl]methyl]amino]-benzoic acid-1-methylethyl ester (CAS 420138-41-6), 4-[[[2-[(4,6-dimethoxy-2-pyrimidinyl)oxy]phenyl]methyl]amino]-benzoic acid propyl ester (CAS 420138-40-5), N-(4-bromophenyl)-2-[(4,6-dimethoxy-2-pyrimidinyl)oxy]benzenemethanamine (CAS 420138-01-8),
sulfonylaminocarbonyl-triazolinone herbicides such as flucarbazone, flucarbazone-sodium, propoxycarbazone, propoxycarbazone-sodium, thiencarbazone and thiencarbazone-methyl; and triafamone;
among these, a preferred embodiment of the invention relates to those compositions comprising at least one imidazolinone herbicide;
b3) from the group of the photosynthesis inhibitors:
amicarbazone, inhibitors of the photosystem II, e.g. 1-(6-tert-butylpyrimidin-4-yl)-2-hydroxy-4-methoxy-3-methyl-2H-pyrrol-5-one (CAS 1654744-66-7), 1-(5-tert-butylisoxazol-3-yl)-2-hydroxy-4-methoxy-3-methyl-2H-pyrrol-5-one (CAS 1637455-12-9), 1-(5-tert-butylisoxazol-3-yl)-4-chloro-2-hydroxy-3-methyl-2H-pyrrol-5-one (CAS 1637453-94-1), 1-(5-tert-butyl-1-methyl-pyrazol-3-yl)-4-chloro-2-hydroxy-3-methyl-2H-pyrrol-5-one (CAS 1654057-29-0), 1-(5-tert-butyl-1-methyl-py-razol-3-yl)-3-chloro-2-hydroxy-4-methyl-2H-pyrrol-5-one (CAS 1654747-80-4), 4-hydroxy-1-methoxy-5-methyl-3-[4-(trifluoromethyl)-2-pyridyl]imidazolidin-2-one; (CAS 2023785-78-4), 4-hydroxy-1,5-dimethyl-3-[4-(trifluoromethyl)-2-pyridyl]imidazolidin-2-one (CAS 2023785-79-5), 5-ethoxy-4-hydroxy-1-methyl-3-[4-(trifluoromethyl)-2-pyridyl]imidazolidin-2-one (CAS 1701416-69-4), 4-hydroxy-1-methyl-3-[4-(trifluoromethyl)-2-pyridyl]imidazolidin-2-one (CAS 1708087-22-2), 4-hydroxy-1,5-dimethyl-3-[1-methyl-5-(trifluoromethyl)pyrazol-3-yl]imidazolidin-2-one (CAS 2023785-80-8), 1-(5-tert-butylisoxazol-3-yl)-4-ethoxy-5-hydroxy-3-methyl-imidazolidin-2-one (CAS 1844836-64-1), triazine herbicides, including of chlorotriazine, triazinones, triazindiones, methylthiotriazines and pyridazinones such as ametryn, atrazine, chloridazone, cyanazine, desmetryn, dimethametryn,hexazinone, metribuzin, prometon, prometryn, propazine, simazine, simetryn, terbumeton, terbuthylazin, terbutryn and trietazin, aryl urea such as chlorobromuron, chlorotoluron, chloroxuron, dimefuron, diuron, fluometuron, isoproturon, isouron, linuron, met-amitron, methabenzthiazuron, metobenzuron, metoxuron, monolinuron, neburon, siduron, tebuthiuron and thiadiazuron, phenyl carbamates such as desmedipham, karbutilat, phenmedipham, phenmedipham-ethyl, nitrile herbicides such as bromofenoxim, bromoxynil and its salts and esters, ioxynil and its salts and esters, uraciles such as bromacil, lenacil and terbacil, and bentazon and bentazon-sodium, pyridate, pyridafol, pentanochlor and propanil and inhibitors of the photosystem I such as diquat, diquat-dibromide, paraquat, paraquat-dichloride and paraquat-dimetilsulfate. Among these, a preferred embodiment of the invention relates to those compositions comprising at least one aryl urea herbicide. Among these, likewise a preferred embodiment of the invention relates to those compositions comprising at least one triazine herbicide. Among these, likewise a preferred embodiment of the invention relates to those compositions comprising at least one nitrile herbicide;
b4) from the group of the protoporphyrinogen-IX oxidase inhibitors:
acifluorfen, acifluorfen-sodium, azafenidin, bencarbazone, benzfendizone, bifenox, butafenacil, carfentrazone, carfentrazone-ethyl, chlomethoxyfen, chlorphthalim, cinidon-ethyl, cyclopyranil, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, tiafenacil, trifludimoxazin, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), N-ethyl-3-(2,6-dichloro-4-trifluoromethylphe-noxy)-5-methyl-1H-pyrazole-1-carboxamide (CAS 452098-92-9), N-tetrahydrofurfuryl-3-(2,6-di-chloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-1-carboxamide (CAS 452099-05-7), N-tetrahydrofurfuryl-3-(2-chloro-6-fluoro-4-trifluoromethylphenoxy)-5-methyl-1H-pyrazole-1-carboxamide (CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0), 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0), methyl (E)-4-[2-chloro-5-[4-chloro-5-(difluoromethoxy)-1H-methyl-pyrazol-3-yl]-4-fluoro-phenoxy]-3-methoxy-but-2-enoate (CAS 948893-00-3), and 3-[7-chloro-5-fluoro-2-(trifluoromethyl)-1H-benzimidazol-4-yl]-1-methyl-6-(trifluoromethyl)-1H-pyrimidine-2,4-dione (CAS 212754-02-4), 2-[2-chloro-5-[3-chloro-5-(trifluoromethyl)-2-pyridinyl]-4-fluorophenoxy]-2-methoxy-acetic acid methyl ester (CAS 1970221-16-9), 2-[2-[[3-chloro-6-[3,6-dihydro-3-methyl-2,6-dioxo-4-(trifluoromethyl)-1(2H)-pyrimidinyl]-5-fluoro-2-pyridinyl]oxy]phenoxy]-acetic acid methyl ester (CAS 2158274-96-3), 2-[2-[[3-chloro-6-[3,6-dihydro-3-methyl-2,6-dioxo-4-(trifluoromethyl)-1(2H)-pyrimidinyl]-5-fluoro-2-pyridinyl]oxy]phenoxy] acetic acid ethyl ester (CAS 158274-50-9), methyl 2-[[3-[2-chloro-5-[4-(difluoromethyl)-3-methyl-5-oxo-1,2,4-triazol-1-yl]-4-fluoro-phenoxy]-2-pyridyl]oxy]acetate (CAS 2271389-22-9), ethyl 2-[[3-[2-chloro-5-[4-(difluoromethyl)-3-methyl-5-oxo-1,2,4-triazol-1-yl]-4-fluoro-phenoxy]-2-pyridyl]oxy]acetate (CAS 2230679-62-4), 2-[[3-[[3-chloro-6-[3,6-dihydro-3-methyl-2,6-dioxo-4-(trifluoromethyl)-1(2H)-pyrimidinyl]-5-fluoro-2-pyridinyl]oxy]-2-pyridinyl]oxy]-acetic acid methyl ester (CAS 2158275-73-9), 2-[[3-[[3-chloro-6-[3,6-dihydro-3-methyl-2,6-dioxo-4-(trifluoromethyl)-1(2H)-pyrimidinyl]-5-fluoro-2-pyridinyl]oxy]-2-pyridinyl]oxy] acetic acid ethyl ester (CAS 2158274-56-5), 2-[2-[[3-chloro-6-[3,6-dihydro-3-methyl-2,6-dioxo-4-(trifluoromethyl)-1(2H)-pyrimidinyl]-5-fluoro-2-pyridinyl]oxy]phenoxy]-N-(methyl-sulfonyl)-acetamide (CAS 2158274-53-2), 2-[[3-[[3-chloro-6-[3,6-dihydro-3-methyl-2,6-dioxo-4-(trifluoromethyl)-1(2H)-pyrimidinyl]-5-fluoro-2-pyridinyl]oxy]-2-pyridinyl]oxy]-N-(methylsulfonyl)-acetamide (CAS 2158276-22-1);
b5) from the group of the bleacher herbicides:
PDS inhibitors: beflubutamid, diflufenican, fluridone, flurochloridone, flurtamone, norflurazon, picolinafen, and 4-(3-trifluoromethylphenoxy)-2-(4-trifluoromethylphenyl)pyrimidine (CAS 180608-33-7), HPPD inhibitors: benzobicyclon, benzofenap, bicyclopyrone, clomazone, fenquinotrione, isoxaflutole, mesotrione, oxotrione (CAS 1486617-21-3), pyrasulfotole, pyrazol-ynate, pyrazoxyfen, sulcotrione, tefuryltrione, tembotrione, tolpyralate, topramezone, bleacher, unknown target: aclonifen, amitrole flumeturon 2-chloro-3-methylsulfanyl-N-(1-methyltetrazol-5-yl)-4-(trifluoromethyl)benzamide (CAS 1361139-71-0), bixlozone and 2-(2,5-dichlorophenyl)me-thyl-4,4-dimethyl-3-isoxazolidinone (CAS 81778-66-7);
b6) from the group of the EPSP synthase inhibitors:
glyphosate, glyphosate-isopropylammonium, glyposate-potassium and glyphosate-trimesium (sulfosate);
b7) from the group of the glutamine synthase inhibitors:
bilanaphos (bialaphos), bilanaphos-sodium, glufosinate, glufosinate-P and glufosinate-ammonium;
b8) from the group of the DHP synthase inhibitors:
asulam;
b9) from the group of the mitosis inhibitors:
compounds of group K1: dinitroanilines such as benfluralin, butralin, dinitramine, ethalfluralin, fluchloralin, oryzalin, pendimethalin, prodiamine and trifluralin, phosphoramidates such as amiprophos, amiprophos-methyl, and butamiphos, benzoic acid herbicides such as chlorthal, chlor-thal-dimethyl, pyridines such as dithiopyr and thiazopyr, benzamides such as propyzamide and tebutam; compounds of group K2: carbetamide, chlorpropham, flamprop, flamprop-isopropyl, flamprop-methyl, flamprop-M-isopropyl, flamprop-M-methyl and propham; among these, compounds of group K1, in particular dinitroanilines are preferred;
b10) from the group of the VLCFA inhibitors:
chloroacetamides such as acetochlor, alachlor, amidochlor, butachlor, dimethachlor, dimethenamid, dimethenamid-P, metazachlor, metolachlor, metolachlor-S, pethoxamid, pretilachlor, propachlor, propisochlor and thenylchlor, oxyacetanilides such as flufenacet and mefenacet, ac-etanilides such as diphenamid, naproanilide, napropamide and napropamide-M, tetrazolinones such fentrazamide, and other herbicides such as anilofos, cafenstrole, fenoxasulfone, ipfen-carbazone, piperophos, pyroxasulfone and isoxazoline compounds of the formulae II.1, II.2, II.3, II.4, II.5, II.6, II.7, II.8 and II.9

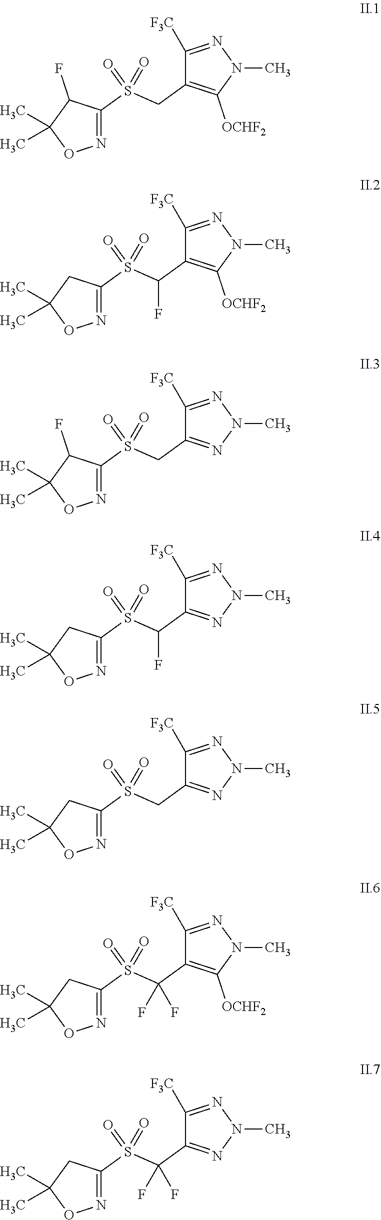

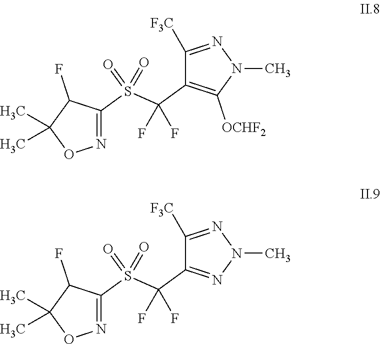

the isoxazoline compounds of the formula (II) are known in the art, e.g. from WO 2006/024820, WO 2006/037945, WO 2007/071900 and WO 2007/096576;
among the VLCFA inhibitors, preference is given to chloroacetamides and oxyacetamides;
b11) from the group of the cellulose biosynthesis inhibitors:
chlorthiamid, dichlobenil, flupoxam, indaziflam, isoxaben, triaziflam and 1-cyclohexyl-5-pen-tafluorphenyloxy-14-[1,2,4,6]thiatriazin-3-ylamine (CAS 175899-01-1);
b12) from the group of the decoupler herbicides:
dinoseb, dinoterb and DNOC and its salts;
b13) from the group of the auxinic herbicides:
2,4-D and its salts and esters such as clacyfos, 2,4-DB and its salts and esters, aminocyclopy-rachlor and its salts and esters, aminopyralid and its salts such as aminopyralid-dimethylammonium, aminopyralid-tris(2-hydroxypropyl)ammonium and its esters, benazolin, benazolin-ethyl, chloramben and its salts and esters, clomeprop, clopyralid and its salts and esters, dicamba and its salts and esters, dichlorprop and its salts and esters, dichlorprop-P and its salts and esters, flopyrauxifen, fluroxypyr, fluroxypyr-butometyl, fluroxypyr-meptyl, halauxifen and its salts and esters (CAS 943832-60-8); MCPA and its salts and esters, MCPA-thioethyl, MCPB and its salts and esters, mecoprop and its salts and esters, mecoprop-P and its salts and esters, piclo-ram and its salts and esters, quinclorac, quinmerac, TBA (2,3,6) and its salts and esters, triclopyr and its salts and esters, florpyrauxifen, florpyrauxifen-benzyl (CAS 1390661-72-9) and 4-amino-3-chloro-5-fluoro-6-(7-fluoro-1H-indol-6-yl)picolinic acid (CAS 1629965-65-6);
b14) from the group of the auxin transport inhibitors: diflufenzopyr, diflufenzopyr-sodium, naptalam and naptalam-sodium;
b15) from the group of the other herbicides: bromobutide, chlorflurenol, chlorflurenol-methyl, cinmethylin, cumyluron, cyclopyrimorate (CAS 499223-49-3) and its salts and esters, dalapon, dazomet, difenzoquat, difenzoquat-metilsulfate, dimethipin, DSMA, dymron, endothal and its salts, etobenzanid, flurenol, flurenol-butyl, flurprimidol, fosamine, fosamine-ammonium, inda-nofan, maleic hydrazide, mefluidide, metam, methiozolin, methyl azide, methyl bromide, methyl-dymron, methyl iodide, MSMA, oleic acid, oxaziclomefone, pelargonic acid, pyributicarb, quinoc-lamine tetflupyrolimet, and tridiphane.

Moreover, it may be useful to apply the compounds of formula (I) in combination with safeners. Safeners are chemical compounds which prevent or reduce damage on useful plants without having a major impact on the herbicidal action of the compounds of the formula (I) towards undesired vegetation. They can be applied either before sowings (e.g. on seed treatments, shoots or seedlings) or in the pre-emergence application or post-emergence application of the useful plant. The safeners and the compounds of formula (I) and optionally the herbicides B can be applied simultaneously or in succession.

In another embodiment of the present invention the combinations according to the present invention comprise at least one compound of formula (I) and at least one safener C (component C).

Examples of safeners are e.g. (quinolin-8-oxy)acetic acids, 1-phenyl-5-haloalkyl-1H-1,2,4-triazol-3-carboxylic acids, 1-phenyl-4,5-dihydro-5-alkyl-1H-pyrazol-3,5-dicarboxylic acids, 4,5-dihydro-5,5-diaryl-3-isoxazol carboxylic acids, dichloroacetamides, alpha-oximinophenylacetonitriles, acetophenonoximes, 4,6-dihalo-2-phenylpyrimidines, N-[[4-(aminocarbonyl)phenyl]sulfonyl]-2-benzoic amides, 1,8-naphthalic anhydride, 2-halo-4-(haloalkyl)-5-thiazol carboxylic acids, phosphorthiolates and N-alkyl-O-phenylcarbamates and their agriculturally acceptable salts and their agriculturally acceptable derivatives such amides, esters, and thioesters, provided they have an acid group.

Examples of safener compounds C are benoxacor, cloquintocet, cyometrinil, cyprosulfamide, dichlormid, dicyclonon, dietholate, fenchlorazole, fenclorim, flurazole, fluxofenim, furilazole, isoxadifen, mefenpyr, mephenate, naphthalic anhydride, oxabetrinil, 4-(dichloroacetyl)-1-oxa-4-azaspiro[4.5]decane (MON4660, CAS 71526-07-3), 2,2,5-trimethyl-3-(dichloroacetyl)-1,3-oxazolidine (R-29148, CAS 52836-31-4), metcamifen and BPCMS (CAS 54091-06-4).

The active compounds B of groups b1) to b15) and the active compounds C are known herbicides and safeners, see, for example, The Compendium of Pesticide Common Names (http://www.alanwood.net/pesticides/); Farm Chemicals Handbook 2000 volume 86, Meister Publishing Company, 2000; B. Hock, C. Fedtke, R. R. Schmidt, Herbizide [Herbicides], Georg Thieme Verlag, Stuttgart 1995; W. H. Ahrens, Herbicide Handbook, 7th edition, Weed Science Society of America, 1994; and K. K. Hatzios, Herbicide Handbook, Supplement for the 7th edition, Weed Science Society of America, 1998. 2,2,5-Trimethyl-3-(dichloroacetyl)-1,3-oxazolidine [CAS No. 52836-31-4] is also referred to as R-29148. 4-(Dichloroacetyl)-1-oxa-4-azaspiro[4.5]decane [CAS No. 71526-07-3] is also referred to as AD-67 and MON 4660.

The assignment of the active compounds to the respective mechanisms of action is based on current knowledge. If several mechanisms of action apply to one active compound, this substance was only assigned to one mechanism of action.

The invention also relates to formulations comprising at least an auxiliary and at least one compound of formula (I) according to the invention.

A formulation comprises a pesticidally effective amount of a compound of formula (I). The term “effective amount” denotes an amount of the combination or of the compound of formula (I), which is sufficient for controlling undesired vegetation, especially for controlling undesired vegetation in crops (i.e. cultivated plants) and which does not result in a substantial damage to the treated crop plants. Such an amount can vary in a broad range and is dependent on various factors, such as the undesired vegetation to be controlled, the treated crop plants or material, the climatic conditions and the specific compound of formula (I) used.

The compounds of formula (I), their salts and thioesters can be converted into customary types of formulations, e.g. solutions, emulsions, suspensions, dusts, powders, pastes, granules, pressings, capsules, and mixtures thereof. Examples for formulation types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further formulation types are defined in the “Catalogue of pesticide formulation types and international coding system”, Technical Monograph No. 2, 6^{th} Ed. May 2008, CropLife International.

The formulations are prepared in a known manner, such as described by Mollet and Grube-mann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetting agents, adjuvants, solubilizers, penetration enhancers, protective colloids, adhesion agents, thickeners, humectants, repellents, attractants, feeding stimulants, compatibil-izers, bactericides, anti-freezing agents, anti-foaming agents, colorants, tackifiers and binders.

Suitable solvents and liquid carriers are water and organic solvents, such as mineral oil frac-tions of medium to high boiling point, e.g. kerosene, diesel oil; oils of vegetable or animal origin; aliphatic, cyclic and aromatic hydrocarbons, e.g. toluene, paraffin, tetrahydronaphthalene, alkyl-ated naphthalenes; alcohols, e.g. ethanol, propanol, butanol, benzylalcohol, cyclohexanol; gly-cols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, lime-stone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. ce-real meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & De-tergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of con-densed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrroli-done, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B—C type comprising alkanol, polyethylene oxide and polypropylene oxide. Suitable polyelectrolytes are polyacids or polybases. Examples of polyacids are alkali salts of poly-acrylic acid or polyacid comb polymers. Examples of polybases are polyvinylamines or polyeth-yleneamines.

Suitable adjuvants are compounds, which have a neglectable or even no pesticidal activity themselves, and which improve the biological performance of the compounds of formula (I) on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, pol-yacrylates, biological or synthetic waxes, and cellulose ethers.

Examples for formulation types and their preparation are:
i) Water-soluble concentrates (SL, LS) 10-60 wt % of a compound of formula (I) or a combination comprising at least one compound of formula (I) (component A) and at least one further compound selected from the herbicidal compounds B (component B) and safeners C (component C) according to the invention and 5-15 wt % wetting agent (e.g. alcohol alkoxylates) are dissolved in water and/or in a water-soluble solvent (e.g. alcohols) ad 100 wt %. The active substance dissolves upon dilution with water.
ii) Dispersible concentrates (DC) 5-25 wt % of a compound of formula (I) or a combination comprising at least one compound of formula (I) (component A) and at least one further compound selected from the herbicidal compounds B (component B) and safeners C (component C) according to the invention and 1-10 wt % dispersant (e.g. polyvinylpyrrolidone) are dissolved in organic solvent (e.g. cyclohexanone) ad 100 wt %. Dilution with water gives a dispersion.
iii) Emulsifiable concentrates (EC) 15-70 wt % of compound of formula (I) or a combination comprising at least one compound of formula (I) (component A) and at least one further compound selected from the herbicidal compounds B (component B) and safeners C (component C) according to the invention and 5-10 wt % emulsifiers (e.g. calcium dodecylbenzenesulfonate and castor oil ethoxylate) are dissolved in water-insoluble organic solvent (e.g. aromatic hydrocarbon) ad 100 wt %. Dilution with water gives an emulsion.
iv) Emulsions (EW, EO, ES)

5-40 wt % of compound of formula (I) or a combination comprising at least one compound of formula (I) (component A) and at least one further compound selected from the herbicidal compounds B (component B) and safeners C (component C) according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is intro-duced into water ad 100 wt % by means of an emulsifying machine and made into a homogene-ous emulsion. Dilution with water gives an emulsion.
v) Suspensions (SC, OD, FS)

In an agitated ball mill, 20-60 wt % of a compound of formula (I) or a combination comprising at least one compound of formula (I) (component A) and at least one further compound selected from the herbicidal compounds B (component B) and safeners C (component C) according to the invention are comminuted with addition of 2-10 wt % dispersants and wetting agents (e.g. sodium lignosulfonate and alcohol ethoxylate), 0,1-2 wt % thickener (e.g. xanthan gum) and water ad 100 wt % to give a fine active substance suspension. Dilution with water gives a stable suspension of the active substance. For FS type formulation up to 40 wt % binder (e.g. polyvinyl-alcohol) is added.
vi) Water-dispersible granules and water-soluble granules (WG, SG)

50-80 wt % of a compound of formula (I) or a combination comprising at least one compound of formula (I) (component A) and at least one further compound selected from the herbicidal compounds B (component B) and safeners C (component C) according to the invention are ground finely with addition of dispersants and wetting agents (e.g. sodium lignosulfonate and alcohol ethoxylate) ad 100 wt % and prepared as water-dispersible or water-soluble granules by means of technical appliances (e.g. extrusion, spray tower, fluidized bed). Dilution with water gives a stable dispersion or solution of the active substance.
vii) Water-dispersible powders and water-soluble powders (WP, SP, WS)

50-80 wt % of a compound of formula (I) or a combination comprising at least one compound of formula (I) (component A) and at least one further compound selected from the herbicidal compounds B (component B) and safeners C (component C) according to the invention are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.
viii) Gel (GW, GF)

In an agitated ball mill, 5-25 wt % of a compound of formula (I) or a combination comprising at least one compound of formula (I) (component A) and at least one further compound selected from the herbicidal compounds B (component B) and safeners C (component C) according to the invention are comminuted with addition of 3-10 wt % dispersants (e.g. sodium lignosulfonate), 1-5 wt % thickener (e.g. carboxymethylcellulose) and water ad 100 wt % to give a fine suspension of the active substance. Dilution with water gives a stable suspension of the active substance.
iv) Microemulsion (ME)

5-20 wt % of a compound of formula (I) or a combination comprising at least one compound of formula (I) (component A) and at least one further compound selected from the herbicidal compounds B (component B) and safeners C (component C) according to the invention are added to 5-30 wt % organic solvent blend (e.g. fatty acid dimethylamide and cyclohexanone), 10-25 wt % surfactant blend (e.g. alcohol ethoxylate and arylphenol ethoxylate), and water ad 100%. This mixture is stirred for 1 h to produce spontaneously a thermodynamically stable microemulsion.
iv) Microcapsules (CS)

An oil phase comprising 5-50 wt % of a compound of formula (I) or a combination comprising at least one compound of formula (I) (component A) and at least one further compound selected from the herbicidal compounds B (component B) and safeners C (component C) according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymeri-zation initiated by a radical initiator results in the formation of poly(meth)acrylate microcapsules. Alternatively, an oil phase comprising 5-50 wt % of a compound of formula (I) according to the invention, 0-40 wt % water insoluble organic solvent (e.g. aromatic hydrocarbon), and an isocya-nate monomer (e.g. diphenylmethene-4,4′-diisocyanate) are dispersed into an aqueous solution of a protective colloid (e.g. polyvinyl alcohol). The addition of a polyamine (e.g. hexamethylene-diamine) results in the formation of polyurea microcapsules. The monomers amount to 1-10 wt %. The wt % relate to the total CS formulation.
ix) Dustable powders (DP, DS)

1-10 wt % of a compound of formula (I) or a combination comprising at least one compound of formula (I) (component A) and at least one further compound selected from the herbicidal compounds B (component B) and safeners C (component C) according to the invention are ground finely and mixed intimately with solid carrier (e.g. finely divided kaolin) ad 100 wt %.
x) Granules (GR, FG)

0.5-30 wt % of a compound of formula (I) or a combination comprising at least one compound of formula (I) (component A) and at least one further compound selected from the herbicidal compounds B (component B) and safeners C (component C) according to the invention is ground finely and associated with solid carrier (e.g. silicate) ad 100 wt %. Granulation is achieved by extrusion, spray-drying or the fluidized bed.
xi) Ultra-low volume liquids (UL)

1-50 wt % of a compound of formula (I) or a combination comprising at least one compound of formula (I) (component A) and at least one further compound selected from the herbicidal compounds B (component B) and safeners C (component C) according to the invention are dissolved in organic solvent (e.g. aromatic hydrocarbon) ad 100 wt %.

The formulation types i) to xi) may optionally comprise further auxiliaries, such as 0,1-1 wt % bactericides, 5-15 wt % anti-freezing agents, 0,1-1 wt % anti-foaming agents, and 0,1-1 wt % colorants.

The formulations and/or combinations generally comprise between 0.01 and 95%, preferably between 0.1 and 90%, and in particular between 0.5 and 75%, by weight of the compounds of formula (I).

The compounds of formula (I) are employed in a purity of from 90% to 100%, preferably from 95% to 100% (according to NMR spectrum).

Solutions for seed treatment (LS), suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. The formulations in question give, after two-to-tenfold dilution, active substance concentrations of from 0.01 to 60% by weight, preferably from 0.1 to 40% by weight, in the ready-to-use preparations. (nach unten verschoben)

Methods for applying compounds of formula (I), formulations and/or combinations thereof, on to plant propagation material, especially seeds, include dressing, coating, pelleting, dusting, soaking and in-furrow application methods of the propagation material. Preferably, compounds of formula (I), formulations and/or combinations thereof, respectively, are applied on to the plant propagation material by a method such that germination is not induced, e.g. by seed dressing, pelleting, coating and dusting.

Various types of oils, wetting agents, adjuvants, fertilizer, or micronutrients, and further pesticides (e.g. herbicides, insecticides, fungicides, growth regulators, safeners) may be added to the compounds of formula (I), the formulations and/or the combinations comprising them as pre-mix or, if appropriate not until immediately prior to use (tank mix). These agents can be admixed with the formulations according to the invention in a weight ratio of 1:100 to 100:1, preferably 1:10 to 10:1.

The user applies the compounds of formula (I) according to the invention, the formulations and/or the combinations comprising them usually from a pre-dosage device, a knapsack sprayer, a spray tank, a spray plane, or an irrigation system. Usually, the formulation is made up with water, buffer, and/or further auxiliaries to the desired application concentration and the ready-to-use spray liquor or the formulation according to the invention is thus obtained. Usually, 20 to 2000 liters, preferably 50 to 400 liters, of the ready-to-use spray liquor are applied per hectare of agricultural useful area.

According to one embodiment, either individual components of the formulation according to the invention or partially premixed components, e.g. components comprising compounds of formula (I) and optionally active substances from the groups B and/or C), may be mixed by the user in a spray tank and further auxiliaries and additives may be added, if appropriate.
In a further embodiment, individual components of the formulation according to the invention such as parts of a kit or parts of a binary or ternary mixture may be mixed by the user himself in a spray tank and further auxiliaries may be added, if appropriate.
In a further embodiment, either individual components of the formulation according to the invention or partially premixed components, e.g components comprising compounds of formula (I) and optionally active substances from the groups B and/or C), can be applied jointly (e.g. after tank mix) or consecutively.

The compounds of formula (I), are suitable as herbicides. They are suitable as such, as an appropriate formulation or in combination with at least one further compound selected from the herbicidal active compounds B (component B) and safeners C (component C).

The compounds of formula (I), or the formulations and/or combinations comprising the compounds of formula (I), control undesired vegetation on non-crop areas very efficiently, especially at high rates of application. They act against broad-leaved weeds and grass weeds in crops such as wheat, rice, maize, soya and cotton without causing any significant damage to the crop plants. This effect is mainly observed at low rates of application.

The compounds of formula (I), or the formulations and/or the combinations comprising them, are applied to the plants mainly by spraying the leaves. Here, the application can be carried out using, for example, water as carrier by customary spraying techniques using spray liquor amounts of from about 100 to 1000 I/ha (for example from 300 to 400 I/ha). The compounds of formula (I), or the formulations and/or the combinations comprising them, may also be applied by the low-volume or the ultra-low-volume method, or in the form of microgranules.

Application of the compounds of formula (I), or the formulations and/or the combinations comprising them, can be done before, during and/or after, preferably during and/or after, the emergence of the undesired vegetation.

Application of the compounds of formula (I), or the formulations and/or the combinations can be carried out before or during sowing.

The compounds of formula (I), or the formulations and/or the combinations comprising them, can be applied pre-, post-emergence or pre-plant, or together with the seed of a crop plant. It is also possible to apply the compounds of formula (I), or the formulations and/or the combinations comprising them, by applying seed, pretreated with the compounds of formula (I), or the formulations and/or the combinations comprising them, of a crop plant. If the active ingredients are less well tolerated by certain crop plants, application techniques may be used in which the combinations are sprayed, with the aid of the spraying equipment, in such a way that as far as possible they do not come into contact with the leaves of the sensitive crop plants, while the active ingredients reach the leaves of undesired vegetation growing underneath, or the bare soil surface (post-directed, lay-by).

In a further embodiment, the compounds of formula (I), or the formulations and/or the combinations comprising them, can be applied by treating seed. The treatment of seeds comprises essentially all procedures familiar to the person skilled in the art (seed dressing, seed coating, seed dusting, seed soaking, seed film coating, seed multilayer coating, seed encrusting, seed dripping and seed pelleting) based on the compounds of formula (I), or the formulations and/or the combinations prepared therefrom. Here, the combinations can be applied diluted or undiluted.

The term “seed” comprises seed of all types, such as, for example, corns, seeds, fruits, tubers, seedlings and similar forms. Here, preferably, the term seed describes corns and seeds. The seed used can be seed of the crop plants mentioned above, but also the seed of transgenic plants or plants obtained by customary breeding methods.

When employed in plant protection, the amounts of active substances applied, i.e. the compounds of formula (I), component B and, if appropriate, component C without formulation auxiliaries, are, depending on the kind of effect desired, from 0.001 to 2 kg per ha, preferably from 0.005 to 2 kg per ha, more preferably from 0.05 to 0.9 kg per ha and in particular from 0.1 to 0.75 kg per ha.

In another embodiment of the invention, the application rate of the compounds of formula (I), component B and, if appropriate, component C, is from 0.001 to 3 kg/ha, preferably from 0.005 to 2.5 kg/ha and in particular from 0.01 to 2 kg/ha of active substance (a.s.).

In another preferred embodiment of the invention, the rates of application of the compounds of formula (I) according to the present invention (total amount of compounds of formula (I)) are from 0.1 g/ha to 3000 g/ha, preferably 10 g/ha to 1000 g/ha, depending on the control target, the season, the target plants and the growth stage.
In another preferred embodiment of the invention, the application rates of the compounds of formula (I) are in the range from 0.1 g/ha to 5000 g/ha and preferably in the range from 1 g/ha to 2500 g/ha or from 5 g/ha to 2000 g/ha.
In another preferred embodiment of the invention, the application rate of the compounds of formula (I) is 0.1 to 1000 g/ha, preferably 1 to 750 g/ha, more preferably 5 to 500 g/ha.

The required application rates of herbicidal compounds B are generally in the range of from 0.0005 kg/ha to 2.5 kg/ha and preferably in the range of from 0.005 kg/ha to 2 kg/ha or 0.01 kg/ha to 1.5 kg/h of a.s.

The required application rates of safeners C are generally in the range of from 0.0005 kg/ha to 2.5 kg/ha and preferably in the range of from 0.005 kg/ha to 2 kg/ha or 0.01 kg/ha to 1.5 kg/h of a.s.

In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.
In another embodiment of the invention, to treat the seed, the amounts of active substances applied, i.e. the compounds of formula (I), component B and, if appropriate, component C are generally employed in amounts of from 0.001 to 10 kg per 100 kg of seed.
When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

In case of combinations according to the present invention it is immaterial whether the compounds of formula (I), and the further component B and/or the component C are formulated and applied jointly or separately.

In the case of separate application, it is of minor importance, in which order the application takes place. It is only necessary, that the compounds of formula (I), and the further component B and/or the component C are applied in a time frame that allows simultaneous action of the active ingredients on the plants, preferably within a time-frame of at most 14 days, in particular at most 7 days.

Depending on the application method in question, the compounds of formula (I), or the formulations and/or combinations comprising them, can additionally be employed in a further number of crop plants for eliminating undesired vegetation. Examples of suitable crops are the following:
Allium cepa, Ananas comosus, Arachis hypogaea, Asparagus officinalis, Avena sativa, Beta vulgaris spec. altissima, Beta vulgaris spec. rapa, Brassica napus var. napus, Brassica napus var. napobrassica, Brassica rapa var. silvestris, Brassica oleracea, Brassica nigra, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N.rustica), Olea europaea, Oryza sativa, Phaseolus lunatus, Phaseolus vulgaris, Picea abies, Pinus spec., Pistacia vera, Pisum sativum, Prunus avium, Prunus persica, Pyrus communis, Prunus armeniaca, Prunus cerasus, Prunus dulcis and Prunus domestica, Ribes sylvestre, Ricinus communis, Saccharum officinarum, Secale cereale, Sinapis alba, Solanum tuberosum, Sorghum bicolor (s. vulgare), Theobroma cacao, Trifolium pratense, Triticum aestivum, Triticale, Triticum durum, Vicia faba, Vitis vinifera and Zea mays.

Preferred crops are Arachis hypogaea, Beta vulgaris spec. altissima, Brassica napus var. napus, Brassica oleracea, Citrus limon, Citrus sinensis, Coffea arabica (Coffea canephora, Coffea liberica), Cynodon dactylon, Glycine max, Gossypium hirsutum, (Gossypium arboreum, Gossypium herbaceum, Gossypium vitifolium), Helianthus annuus, Hordeum vulgare, Juglans regia, Lens culinaris, Linum usitatissimum, Lycopersicon lycopersicum, Malus spec., Medicago sativa, Nicotiana tabacum (N.rustica), Olea europaea, Oryza sativa, Phaseolus lunatus, Phaseolus vulgaris, Pistacia vera, Pisum sativum, Prunus dulcis, Saccharum officinarum, Secale cereale, Solanum tuberosum, Sorghum bicolor (s. vulgare), Triticale, Triticum aestivum, Triticum durum, Vicia faba, Vitis vinifera and Zea mays.

Especially preferred crops are crops of cereals, corn, soybeans, rice, oilseed rape, cotton, potatoes, peanuts or permanent crops.

The compounds of formula (I) according to the invention, or the formulations and/or combinations comprising them, can also be used in crops which have been modified by mutagenesis or genetic engineering in order to provide a new trait to a plant or to modify an already present trait.

The term “crops” as used herein includes also (crop) plants which have been modified by mutagenesis or genetic engineering in order to provide a new trait to a plant or to modify an already present trait.

Mutagenesis includes techniques of random mutagenesis using X-rays or mutagenic chemicals, but also techniques of targeted mutagenesis, in order to create mutations at a specific locus of a plant genome. Targeted mutagenesis techniques frequently use oligonucleotides or proteins like CRISPR/Cas, zinc-finger nucleases, TALENs or meganucleases to achieve the tar-geting effect.

Genetic engineering usually uses recombinant DNA techniques to create modifications in a plant genome which under natural circumstances cannot readily be obtained by cross breeding, mutagenesis or natural recombination. Typically, one or more genes are integrated into the genome of a plant in order to add a trait or improve a trait. These integrated genes are also referred to as transgenes in the art, while plant comprising such transgenes are referred to as transgenic plants. The process of plant transformation usually produces several transformation events, which differ in the genomic locus in which a transgene has been integrated. Plants comprising a specific transgene on a specific genomic locus are usually described as comprising a specific “event”, which is referred to by a specific event name. Traits which have been intro-duced in plants or have been modified include in particular herbicide tolerance, insect resistance, increased yield and tolerance to abiotic conditions, like drought.

Herbicide tolerance has been created by using mutagenesis as well as using genetic engineering. Plants which have been rendered tolerant to acetolactate synthase (ALS) inhibitor herbicides by conventional methods of mutagenesis and breeding comprise plant varieties commercially available under the name Clearfield®. However, most of the herbicide tolerance traits have been created via the use of transgenes.
Herbicide tolerance has been created to glyphosate, glufosinate, 2,4-D, dicamba, oxynil herbicides, like bromoxynil and ioxynil, sulfonylurea herbicides, ALS inhibitor herbicides and 4-hydroxyphenylpyruvate dioxygenase (HPPD) inhibitors, like isoxaflutole and mesotrione.
Transgenes which have been used to provide herbicide tolerance traits comprise: for tolerance to glyphosate: cp4 epsps, epsps grg23ace5, mepsps, 2mepsps, gat4601, gat4621 and goxv247, for tolerance to glufosinate: pat and bar, for tolerance to 2,4-D: aad-1 and aad-12, for tolerance to dicamba: dmo, for tolerance to oxynil herbicies: bxn, for tolerance to sulfonylurea herbicides: zm-hra, csr1-2, gm-hra, S4-HrA, for tolerance to ALS inhibitor herbicides: csr1-2, for tolerance to HPPD inhibitor herbicides: hppdPF, W336 and avhppd-03.
Transgenic corn events comprising herbicide tolerance genes are for example, but not excluding others, DAS40278, MON801, MON802, MON809, MON810, MON832, MON87411, MON87419, MON87427, MON88017, MON89034, NK603, GA21, MZHGOJG, HCEM485, VCO-Ø1981-5, 676, 678, 680, 33121, 4114, 59122, 98140, Bt10, Bt176, CBH-351, DBT418, DLL25, MS3, MS6, MZIR098, T25, TC1507 and TC6275.
Transgenic soybean events comprising herbicide tolerance genes are for example, but not excluding others, GTS 40-3-2, MON87705, MON87708, MON87712, MON87769, MON89788, A2704-12, A2704-21, A5547-127, A5547-35, DP356043, DAS44406-6, DAS68416-4, DAS-81419-2, GU262, SYHTOH2, W62, W98, FG72 and CV127.
Transgenic cotton events comprising herbicide tolerance genes are for example, but not excluding others, 19-51a, 31707, 42317, 81910, 281-24-236, 3006-210-23, BXN10211, BXN10215, BXN10222, BXN10224, MON1445, MON1698, MON88701, MON88913, GHB119, GHB614, LLCotton25, T303-3 and T304-40.
Transgenic canola events comprising herbicide tolerance genes are for example, but not excluding others, MON88302, HCR-1, HCN10, HCN28, HCN92, MS1, MS8, PHY14, PHY23, PHY35, PHY36, RF1, RF2 and RF3.

Insect resistance has mainly been created by transferring bacterial genes for insecticidal proteins to plants. Transgenes which have most frequently been used are toxin genes of Bacillus spec. and synthetic variants thereof, like cry1A, cry1Ab, cry1Ab-Ac, cry1Ac, cry1A.105, cry1F, cry1Fa2, cry2Ab2, cry2Ae, mcry3A, ecry3.1Ab, cry3Bb1, cry34Ab1, cry35Ab1, cry9C, vip3A(a), vip3Aa20. However, also genes of plant origin have been transferred to other plants. In particular genes coding for protease inhibitors, like CpTI and pinll. A further approach uses transgenes in order to produce double stranded RNA in plants to target and downregulate insect genes. An example for such a transgene is dvsnf7.
Transgenic corn events comprising genes for insecticidal proteins or double stranded RNA are for example, but not excluding others, Bt10, Bt11, Bt176, MON801, MON802, MON809, MON810, M0N863, M0N87411, M0N88017, M0N89034, 33121, 4114, 5307, 59122, TC1507, TC6275, CBH-351, MIR162, DBT418 and MZIR098.
Transgenic soybean events comprising genes for insecticidal proteins are for example, but not excluding others, MON87701, MON87751 and DAS-81419.
Transgenic cotton events comprising genes for insecticidal proteins are for example, but not excluding others, SGK321, MON531, MON757, MON1076, MON15985, 31707, 31803, 31807, 31808, 42317, BNLA-601, Event1, COT67B, COT102, T303-3, T304-40, GFM Cry1A, GK12, MLS 9124, 281-24-236, 3006-210-23, GHB119 and SGK321.

Increased yield has been created by increasing ear biomass using the transgene athb17, being present in corn event MON87403, or by enhancing photosynthesis using the transgene bbx32, being present in the soybean event MON87712.

Crops comprising a modified oil content have been created by using the transgenes: gm-fad2-1, Pj.D6D, Nc.Fad3, fad2-1A and fatb1-A. Soybean events comprising at least one of these genes are: 260-05, MON87705 and MON87769.

Tolerance to abiotic conditions, in particular to tolerance to drought, has been created by using the transgene cspB, comprised by the corn event MON87460 and by using the transgene Hahb-4, comprised by soybean event IND-00410-5.

Traits are frequently combined by combining genes in a transformation event or by combining different events during the breeding process. Preferred combination of traits are herbicide tolerance to different groups of herbicides, insect tolerance to different kind of insects, in particular tolerance to lepidopteran and coleopteran insects, herbicide tolerance with one or several types of insect resistance, herbicide tolerance with increased yield as well as a combination of herbicide tolerance and tolerance to abiotic conditions.

Plants comprising singular or stacked traits as well as the genes and events providing these traits are well known in the art. For example, detailed information as to the mutagenized or integrated genes and the respective events are available from websites of the organizations “International Service for the Acquisition of Agri-biotech Applications (ISAAA)” (http://www.isaaa.org/gmapprovaldatabase) and the “Center for Environmental Risk Assess-ment (CERA)” (http://cera-gmc.org/GMCropDatabase), as well as in patent applications, like EP3028573 and WO2017/011288.

The use of the compounds of formula (I) or formulations or combinations comprising them according to the invention on crops may result in effects which are specific to a crop comprising a certain gene or event. These effects might involve changes in growth behavior or changed resistance to biotic or abiotic stress factors. Such effects may in particular comprise enhanced yield, enhanced resistance or tolerance to insects, nematodes, fungal, bacterial, mycoplasma, viral or viroid pathogens as well as early vigor, early or delayed ripening, cold or heat tolerance as well as changed amino acid or fatty acid spectrum or content.

Furthermore, plants are also covered that contain by the use of recombinant DNA techniques a modified amount of ingredients or new ingredients, specifically to improve raw material produc-tion, e.g., potatoes that produce increased amounts of amylopectin (e.g. Amflora® potato, BASF SE, Germany).

Furthermore, it has been found that the compounds of formula (I) according to the invention, or the formulations and/or combinations comprising them, are also suitable for the defoliation and/or desiccation of plant parts of crops such as cotton, potato, oilseed rape, sunflower, soybean or field beans, in particular cotton. In this regard, formulations and/or combinations for the desiccation and/or defoliation of crops, processes for preparing these formulations and/or combinations and methods for desiccating and/or defoliating plants using the compounds of formula (I) have been found.

As desiccants, the compounds of formula (I) are particularly suitable for desiccating the above-ground parts of crop plants such as potato, oilseed rape, sunflower and soybean, but also cereals. This makes possible the fully mechanical harvesting of these important crop plants.

Also of economic interest is to facilitate harvesting, which is made possible by concentrating within a certain period of time the dehiscence, or reduction of adhesion to the tree, in citrus fruit, olives and other species and varieties of pernicious fruit, stone fruit and nuts. The same mechanism, i.e. the promotion of the development of abscission tissue between fruit part or leaf part and shoot part of the plants is also essential for the controlled defoliation of useful plants, in particular cotton.

Moreover, a shortening of the time interval in which the individual cotton plants mature leads to an increased fiber quality after harvesting.

### A Chemistry Examples

Chemical bonds, drawn as bars in chemical formulae, indicate the relative stereochemistry on the ring system.

### EXAMPLE 1

Synthesis of ethyl 4-(3,5-difluorophenyl)-2,3-dihydrofuran-2-carboxylate (Cpd I)

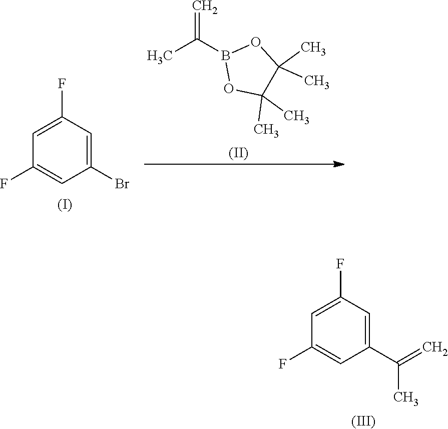

To a mixture of aryl bromide (1) (40 g, 209 mmol) in dimethoxyethane (500 mL) was added compound 11 (35.2 g, 209 mmol), aq. sat, Na₂CO₃ (500 mL) and tetrakis(triphenylphosphine)-palladium(0) (Pd(PPh₃)₄, CAS: 14221-01-3 (7.26 g, 6.28 mmol) at 15° C. and stirred at 90° C. for 16 h under nitrogen atmosphere. The mixture was poured into water (500 mL) and extracted with EtOAc (2×500 mL). The combined organics were washed with brine, dried and concentrated. The crude was purified by flash column chromatography (hexane/EtOAc=9:1) to give compound III (26 g, 81%) as yellow oil. ¹H-NMR (400 MHz, CDC₃): δ=6.97 (dd, J=9.1, 2.1 Hz, 2H), 6.72 (tt, J=8.8, 2.3 Hr, 1H), 5.41 (s, 1H), 2.12 (s, 3H).

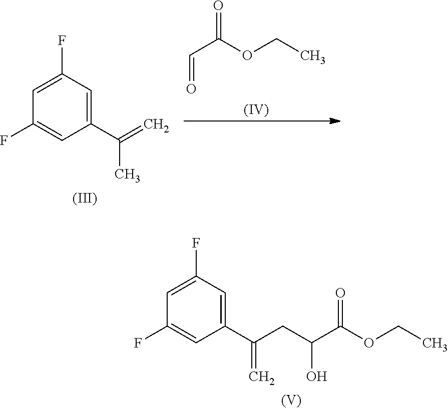

To a mixture of compound III (20 g, 129 mmol) in acetonitrile (200 mL) was added glyoxylic acid ethyl ester (40 g, 389 mmol) and Yb(OTf)₃ (16 g, 25.67 mmol) at 15° C. and stirred at the same temparature for 16 h. The mixture was concentrated, diluted with H₂O (200 mL) and extracted with EtOAc (2×200 mL). The combined organics were washed with brine, dried and concentrated. The crude was purified by flash column chromatography (hexane/EtOAc=9:1) to give compound V (14 g, 42%) as a yellow oil. ¹H-NMR (400 MHz, CDC₃): δ=6.98-6.92 (m, 2H), 6.74 (tt, J=8.8, 2.3 Hz, 1H), 5.45 (s, 1H), 5.30 (d, J=5.0 Hz, 1H), 4.27 (ddd, J=7.4, 5.9, 4.6 Hz, 1H), 4.22-4.10 (m, 2H), 3.00 (dd, J=14.6, 4.3 Hz, 1H), 2.83-2.73 (m, 2H), 1.28 (t, J=7.1 Hz, 3H).

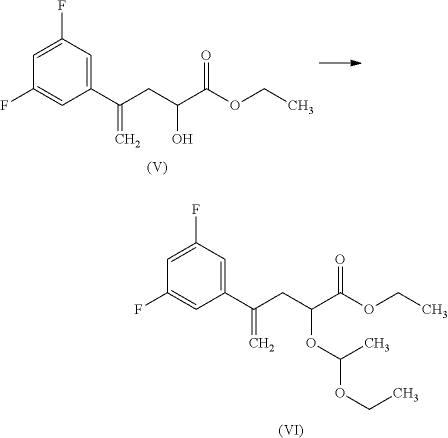

To a mixture of compound V (14 g, 55 mmol) in ethyl vinyl ether (105 mL) was added trifluoroacetic acid (21 mL) at 15° C. and stirred at 50° C. for 16 h. After concentrating the mixture, the crude was purified by flash column chromatography (hexane/EtOAc=10:1) to afford compound VI (12.5 g, 69%) as a yellow oil. ¹H-NMR (400 MHz, CDC₃): δ=7.02-6.91 (m, 2H), 6.80-6.69 (m, 1H), 5.44 (d, J=7.3 Hz, 1H), 5.31-5.25 (m, 1H), 4.76-4.84 (m, 1H), 4.32-4.05 (m, 4H), 3.64-3.31 (m, 2H), 2.98-2.78 (m, 2H), 1.31-1.25 (m, 5H), 1.12-1.02 (m, 3H).

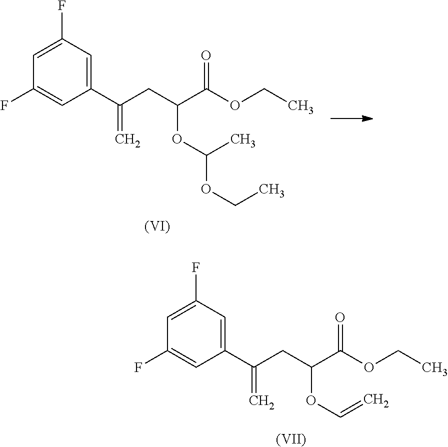

To a solution of compound VI (12.5 g, 36.6 mmol) in dichloromethane (130 mL) was added triethylamine (7.6 mL, 55 mmol) and trimethylsilyl triflate (8.75 mL, 47.5 mmol) at 0° C. und a nitrogen atmosphere. After stirring for 16 h at room temperature, the mixture was diluted with water (100 mL) and extracted with dichloromethane (2×100 mL). The combined extracts were washed with brine, dried over Na₂SO₄ and concentrated. The residue was purified by flash column chromatography (hexane/EtOAc=10:1) to afford compound VII (7.2 g, 60%) as a yellow oil. ¹H-NMR (400 MHz, CDC₃): δ=6.99-6.87 (m, 2H), 6.76-6.71 (m, 1H), 6.34 (dd, J=14.3, 6.8 Hz, 1H), 5.44 (s, 1H), 5.28 (s, 1H), 4.37-4.32 (m, 1H), 4.25-4.13 (m, 3H), 4.07 (dd, J=6.8, 2.5 Hz, 1H), 3.01-2.93 (m, 2H), 1.27 (t, J=7.1 Hz, 3H).

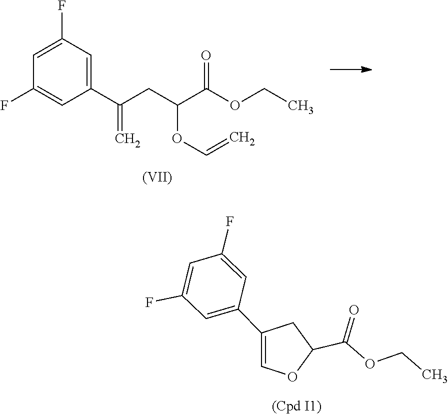

To a solution of compound VII (2.0 g, 6.1 mmol) in 1,2-dichloroethane (1 L) was added Grubb's second generation catalyst (CAS: 301224-40-8) (2.0 g, 2.4 mmol) at 0° C. under a nitrogen atmosphere. After stirring for 16 h at 90° C. under nitrogen, the mixture was diluted with water (10 mL) and stirred for 30 min at room temperature. After concentrating the mixture, the residue was purified by flash column chromatography (hexane/EtOAc=10:1) to afford compound Cpd 11 (3.0 g, 64%) as a yellow oil. ¹H-NMR (400 MHz, CDCl₃): δ=6.93 (s, 1H), 6.76-6.69 (m, 2H), 6.65-6.58 (m, 1H), 5.16 (dd, J=11.5, 7.3 Hz, 1H), 4.29 (q, J=7.2 Hz, 2H), 3.34-3.25 (m, 1H), 3.11 (ddd, J=14.8, 7.2, 1.8 Hz, 1H), 1.34 (t, J=7.1 Hz, 3H).

### EXAMPLE 2

Synthesis of ethyl 4-(3,5-difluorophenyl)-2-methyl-3H-furan-2-carboxylic acid (Cpd I2)

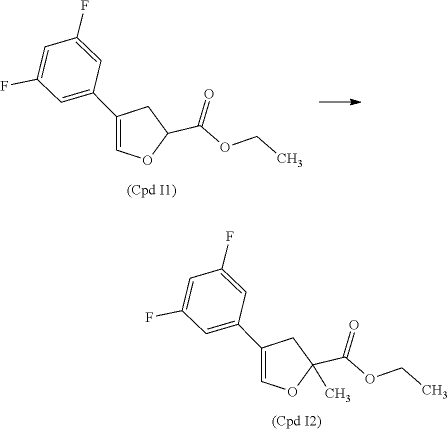 [SMILES: CCOC(=O)C1CC(c2cc(F)cc(F)c2)=CO1 ; CCOC(=O)C1(C)CC(c2cc(F)cc(F)c2)=CO1]

To a solution of Cpd I1 (2.0 g, 7.9 mmol) in THF (100 mL) was added methyl iodide (5.6 g, 39 mmol) and a solution of lithium bis(trimethylsilyl)amide (1 M in THF, 23.6 mL, 23.6 mmol) at 0° C. under a nitrogen atmosphere. After stirring for 2 h at 0° C. under nitrogen, the mixture was poured into water (50 mL), acidified with aq. HCl (1 M) to pH=3 and extracted with EtOAc (2×100 mL). The combined extracts were washed with brine, dried over Na₂SO₄ and concentrated. The residue was purified by flash column chromatography (hexane/EtOAc=10:1) to afford compound Cpd I2 (1.0 g, 47%) as a yellow oil. ¹H-NMR (400 MHz, CDCl₃): δ=6.87 (t, J=1.8 Hz, 1H), 6.74-6.69 (m, 2H), 6.63-6.59 (m, 1H), 4.31-4.23 (m, 2H), 3.36 (dd, J=14.9, 2.0 Hz, 1H), 2.84 (dd, J=14.9, 2.0 Hz, 1H), 1.68 (s, 3H), 1.33 (t, J=7.2 Hz, 3H).

### EXAMPLE 3

Synthesis of 4-(3,5-difluorophenyl)-2-methyl-3H-furan-2-carboxylic acid (Cpd I3)

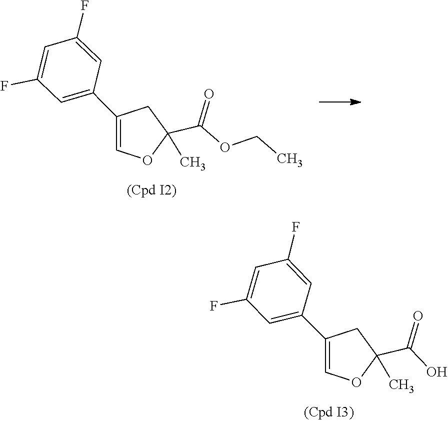 [SMILES: CCOC(=O)C1(C)CC(c2cc(F)cc(F)c2)=CO1 ; CC1(C(=O)O)CC(c2cc(F)cc(F)c2)=CO1]

To a solution of compound Cpd I2 (0.78 g, 2.9 mmol) in THF (9 mL) was added lithium hydroxide hydrate (367 mg, 8.73 mmol) and water (3 mL). After stirring for 2 h at room temperature, the mixture was diluted with water (10 mL), acidified with aq. HCl (1 M) until pH=3 and extracted with EtOAc (3×10 mL). The combined extracts were washed with brine, dried over Na₂SO₄ and concentrated to provide compound Cpd I3 (1.0 g, quantitative) as a yellow oil. This product was used without further purification in the next step. LC-MS (M+H)⁺:240.0.

### EXAMPLE 4

Synthesis of ethyl 4-(3,5-dichlorophenyl)-2-methyl-3H-furan-2-carboxylic acid (Cpd I4):

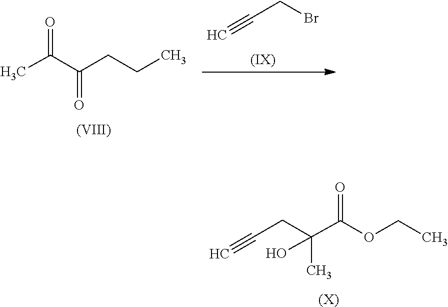

To a solution of ethyl pyruvate (VIII) (50 g, 431 mmol) in THF (200 mL) was added propargyl bromide (IX) (103 g, 862 mmol) and Zn (64.5 g, 1.08 mol) at room temperature. After heating the reaction to 80° C., the suspension was stirred for 3 h. After cooling to room temperature, the mixture was filtered and the filtrate was quenched with HCl (2N) and extracted with EtOAc. The combined organics were washed with brine, dried over Na₂SO₄ and concentrated. The crude was purified by flash column chromatography (hexane/EtOAc=100:0 to 7:3) to give the compound X (34.6 g, 51.6%) as a yellow oil. ¹H-NMR (400 MHz, CDCl₃): δ=4.36-4.16 (m, 2H), 2.72-2.50 (m, 2H), 2.06 (t, J=2.57 Hz, 1H), 1.47 (s, 3H), 1.34-1.28 (m, 3H).

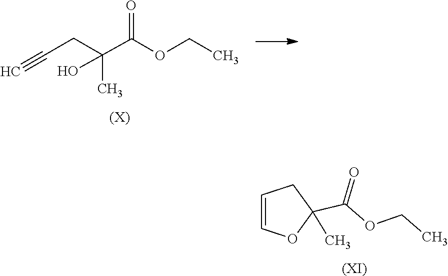

To a mixture of compound X (17 g, 110 mmol) in acetone (300 ml) was added Ag₂0 (12.6 g, 55 mmol) and triethylamine (11.1 g, 110 mmol) at room temperature. After stirring the reaction at 50° C. for 2 h, the suspension was filtered and the filtrate was concentrated. The crude was purified by flash column chromatography (hexane/EtOAc=100:0 to 1:1) to give the compound XI (17 g, 100%) as a yellow oil. ¹H-NMR (400 MHz, CDCl₃): δ=6.30 (q, J=2.4 Hz, 1H), 4.87 (q, J=2.5 Hz, 1H), 4.30-4.18 (m, 2H), 3.04 (dt, J=15.7, 2.3 Hz, 1H) 2.54 (dt, J=15.7, 2.32 Hz, 1H), 1.58 (s, 3H), 1.29-1.33 (t, 3H).

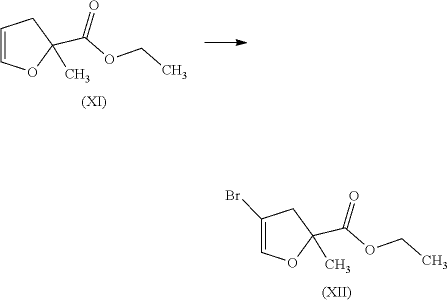

To a solution of compound XI (17 g, 110 mmol) in dichloromethane (250 mL) was added bromine (17.4 g, 110 mmol) in dichloromethane (50 mL) dropwise at −78° C. and stirred at −78° C. for 10 min. 1,8-Diazabicyclo[5.4.0]undec-7-ene (DBU, CAS: 6674-22-2) (67 g, 440 mmol) was added at −78° C. dropwise. After removing the cold bath, the mixture was stirred for 1 h at room temperature. The mixture was quenched with HCl (1 M) and extracted with dichloromethane (2×50 mL). The combined extracts were washed with brine, dried over Na₂SO₄ and concentrated. The crude was purified by flash column chromatography (hexane/EtOAc=100:0 to 1:1) to give the compound XII (16.5 g, 65%) as a yellow amorphous solid. ¹H-NMR (400 MHz, CDCl₃): δ=6.37 (t, J=2.1 Hz, 1H), 4.32-4.20 (m, 2H), 3.26 (dd, J=15.3, 2.1 Hz, 1H), 2.74 (dd, J=15.4, 2.2 Hz, 1H), 1.62 (s, 3H), 1.32 (t, J=7.15 Hz, 3H).

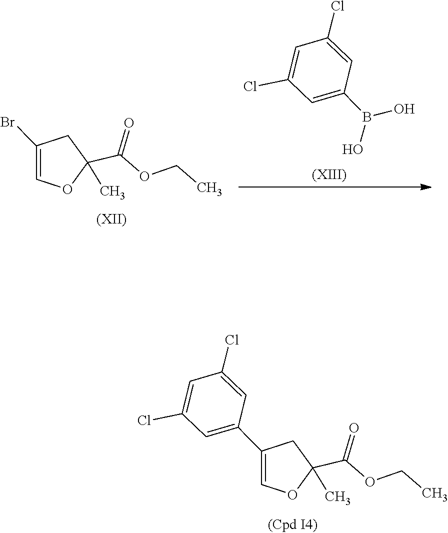

To the emulsion of compound XII (1.5 g, 6.4 mmol) in a 5:1-mixture of toluene (30 mL) and water (6 mL), aryl boronic acid XIII (1.35 g, 7.05 mmol), Cs₂CO₃ (10 g, 32 mmol) and Pd(dppf)Cl₂ (CAS: 72287-26-4) (300 mg, 0.41 mmol) were added at room temperature and the mixture was stirred at 110° C. for 2 h under nitrogen atmosphere. The reaction was quenched with H₂O (20 mL) and extracted with EtOAc (3×30 mL). The combined organics were washed with brine, dried over Na₂SO₄ and concentrated. The crude was purified by flash column chromatography (hexane/EtOAc=100:0 to 1:1) to provide compound Cpd I4 (1.3 g, 68%) as a yellow oil. ¹H-NMR (400 MHz, CDCl₃): δ=7.14 (t, J=1.6 Hz, 1H), 7.07 (d, J=1.6 Hz, 2H), 6.88 (s, 1H), 4.27 (q, J=7.3 Hz, 2H), 3.36 (dd, J=14.9, 1.9 Hz, 1H), 2.84 (dd, J=14.9, 1.9 Hz, 1H), 1.67 (s, 3H), 1.33 (t, J=7.2 Hz, 3H).

### EXAMPLE 5

Synthesis of 4-(3,5-dichlorophenyl)-2-methyl-3H-furan-2-carboxylic acid (Cpd I5):

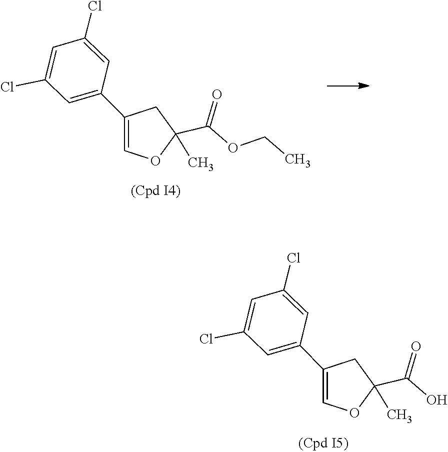 [SMILES: CCOC(=O)C1(C)CC(c2cc(Cl)cc(Cl)c2)=CO1 ; CC1(C(=O)O)CC(c2cc(Cl)cc(Cl)c2)=CO1]

To a solution of compound Cpd I4 (1.3 g, 4.3 mmol) in a 3:1 mixture of THF (15 mL) and water (5 mL) was added lithium hydroxide (364 mg, 3.34 mmol) at room temperature. After stirring for 2 h, the mixture was quenched with H₂O, acidified with aq. HCl (6 M) until pH=3 and extracted with EtOAc (3×20 mL). The combined organics were washed with brine, dried over Na₂SO₄ and concentrated to give Cpd I5 (900 mg, 77%) as an amorphous yellow solid. The crude was used without further purification. For analytic purposes a small sample of Cpd I5 was purified by prep-HPLC (TFA,CH₃CN—H₂O). ¹H-NMR (400 MHz, DMSO-d₆): δ=13.09 (br s, 1H), 7.44 (s, 1 H), 7.35 (d, J=1.6 Hz, 2H), 7.31 (s, 1H), 3.24 (br d, J=13.8 Hz, 1H), 2.85 (br d, J=13.7 Hz, 1H), 1.53 (s, 3H).

### EXAMPLE 6

Synthesis of ethyl 4-(3,5-difluorophenyl)-2-(trifluoromethyl)-3H-furan-2-carboxylic acid (Cpd I6)

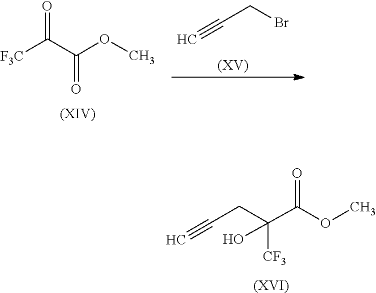

According to the synthesis of Inter C, to a solution of compound XIV (20 g, 13 mmol) in THF (200 mL) was added propargyl bromide (XV) (30.5 g, 256 mmol) and Zn (20.5 g, 321 mmol) at room temperature. After stirring for 2 h, the mixture was filtered, poured into water (100 mL), acidified with HCl (6 M) to pH=3 and extracted with methyl tert-butyl ether (3×100 mL). The combined extracts were washed with brine, dried over Na₂SO₄ and concentrated. The crude was purified by flash column chromatography (hexane/EtOAc=100:0 to 0:100) to give the compound XVI (36 g, 40%) as a yellow oil. The analytical and spectroscopic data are in alignment with the reported data from Tetrahedron 2003, 59, 1389-1394.

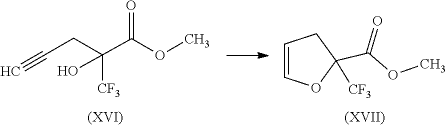

To a mixture of compound XVI (36 g, 0.18 mol) in acetone (700 ml) was added Ag₂0 (21.3 g, 91.8 mmol) and triethylamine (25.5 mL, 114 mmol) at room temperature. After stirring the reaction for 2 h in the dark, the suspension was filtered and the filtrate was concentrated. The crude was purified by flash column chromatography (hexane/EtOAc=100:0 to 0:100) to give the compound XVII (20 g, 55%) as a yellow oil. ¹H-NMR (400 MHz, CDC₃): δ=6.37 (d, J=2.3 Hz, 1H), 5.03 (d, J=2.3 Hz, 1H), 3.89 (s, 3H), 3.13 (d, J=2.4 Hz, 2H).

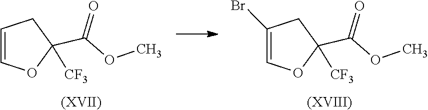

To a solution of compound XVII (15 g, 77 mmol) in dichloromethane (200 mL) was added bromine (12 g, 77 mmol) dropwise at −78° C. and stirred at −78° C. for 15 min. 1,8-Diazabicyclo[5.4.0]undec-7-ene (DBU, CAS: 6674-22-2) (46.5 g, 306 mmol) was added at −78° C. dropwise and stirred for 1 h at the same temperature. The mixture was poured into water (100 mL), acidified with HCl (6 M) to pH=3 and extracted with dichloromethane (2×50 mL). The combined extracts were washed with brine, dried over Na₂SO₄ and concentrated. The crude (8.5 g) was used in the next step without further purification.

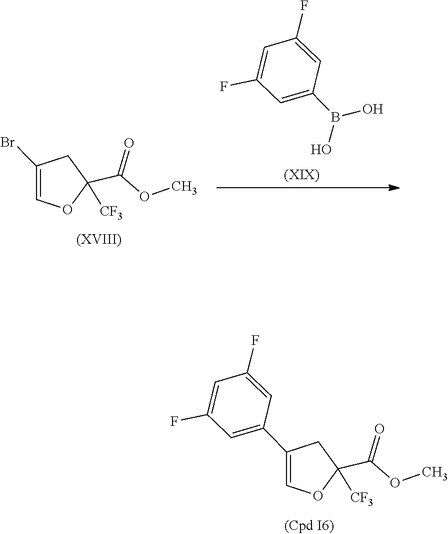

To the emulsion of compound XVIII (7.5 g, 27 mmol) in a 5:1-mixture of toluene (80 mL) and water (16 mL), aryl boronic acid XIX (4.3 g, 27 mmol), Cs₂CO₃ (44.9 g, 137 mmol) and Pd(dppf)Cl₂ (CAS: 72287-26-4) (1.28 g, 1.75 mmol) were added at room temperature and the mixture was stirred at 110° C. for 1 h under nitrogen atmosphere. The reaction was quenched with H₂O (40 mL) and extracted with EtOAc (3×30 mL). The combined organics were washed with brine, dried over Na₂SO₄ and concentrated. The crude (1.8 g, 7%) was used in the next step without further purification. For analytic purposes a small sample of compound Cpd I6 (55 mg) was purified by prep-HPLC (TFA,CH₃CN—H₂O). ¹H-NMR (400 MHz, CDC₃): δ=6.91 (s, 1H), 6.77-6.72 (m, 2H), 6.68 (tt, J=8.8, 2.1 Hz, 1H), 3.92 (s, 3H), 3.50-3.36 (m, 2H).

### EXAMPLE 7

Synthesis of 4-(3,5-difluorophenyl)-2-(trifluoromethyl)-3H-furan-2-carboxylic acid (Cpd I7)

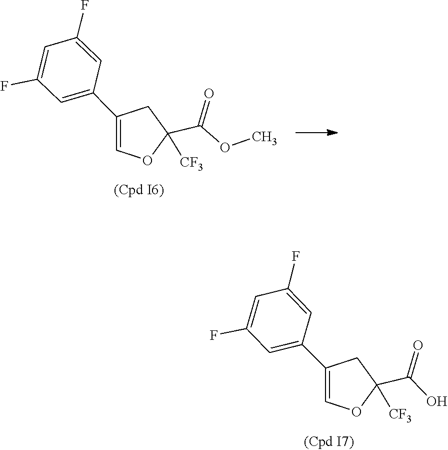 [SMILES: COC(=O)C1(C(F)(F)F)CC(c2cc(F)cc(F)c2)=CO1 ; O=C(O)C1(C(F)(F)F)CC(c2cc(F)cc(F)c2)=CO1]

To a solution of compound Cpd I6 (1.5 g, 4.9 mmol) in a 3:1 mixture of THF (15 mL) and water (5 mL) was added lithium hydroxide (0.31 g, 7.3 mmol) at room temperature. After stirring for 2 h, the mixture was quenched with H₂O, acidified with aq. HCl (6 M) until pH=3 and extracted with EtOAc (3×20 mL). The combined organics were washed with brine, dried over Na₂SO₄ and concentrated to give Cpd I7 (900 mg, 77%) as an amorphous yellow solid. The crude (1.5 g, 21%) was used in the next step without further purification. For analytic purposes a small sample of Cpd I7 was purified by prep-HPLC (TFA,CH₃CN—H₂O). ¹H-NMR (400 MHz, CDCl₃): δ=7.59 (s, 1H), 7.16 (br dd, J=9.3, 2.2 Hz, 2H), 7.05 (tt, J=9.3, 2.1 Hz, 1H), 3.45 (d, J=1.8 Hz, 2H).

High Performance Liquid Chromatography: HPLC-column Kinetex XB C18 1,7μ (50×2.1 mm); eluent: acetonitrile/water+0.1% trifluoroacetic acid (gradient from 5:95 to 100:0 in 1.5 min at 60° C., flow gradient from 0.8 to 1.0 ml/min in 1.5 min).

In analogy to the examples described above, the following compounds of formula (I), wherein R⁸, R⁹ and R¹⁰ are hydrogen, were prepared using commercially available alcohols:

TABLE 2

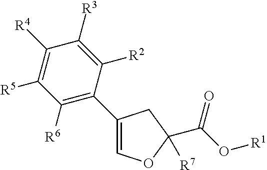

| Cpd. | R¹ | R² | R³ | R⁴ | R⁵ | R⁶ | R⁷ | HPLC/MS |
|---|---|---|---|---|---|---|---|---|
| I1 | CH₂CH₃ | H | F | H | F | H | H | 255.1 |
| I2 | CH₂CH₃ | H | F | H | F | H | CH₃ | 269.1 |
| I3 | H | H | F | H | F | H | CH₃ | 241 |
| I4 | CH₂CH₃ | H | Cl | H | Cl | H | CH₃ | 301 |
| I5 | H | H | Cl | H | Cl | H | CH₃ | 273 |
| I6 | CH₃ | H | F | H | F | H | CF₃ | 309 |
| I7 | H | H | F | H | F | H | CF₃ | 293 |

HPLC/MS = MassChargeRatio

### B Use Examples

The herbicidal activity of the compounds of formula (I) was demonstrated by the following greenhouse experiments:

The culture containers used were plastic flowerpots containing loamy sand with approximately 3.0% of humus as the substrate. The seeds of the test plants were sown separately for each species.

For the pre-emergence treatment, the active ingredients, which had been suspended or emulsified in water, were applied directly after sowing by means of finely distributing nozzles. The containers were irrigated gently to promote germination and growth and subsequently covered with transparent plastic hoods until the test plants had rooted. This cover caused uniform germination of the test plants, unless this had been impaired by the active ingredients. For the post-emergence treatment, the test plants were first grown to a height of 3 to 15 cm, depending on the plant habit, and only then treated with the active ingredients which had been suspended or emulsified in water. For this purpose, the test plants were either sown directly and grown in the same containers, or they were first grown separately as seedlings and transplanted into the test containers a few days prior to treatment.

Depending on the species, the test plants were kept at 10-25° C. or 20-35° C., respectively. The test period extended over 2 to 4 weeks. During this time, the test plants were tended, and their response to the individual treatments was evaluated.

Evaluation was carried out using a scale from 0 to 100. 100 means no emergence of the test plants, or complete destruction of at least the aerial moieties, and 0 means no damage, or normal course of growth. A good herbicidal activity is given at values of 65 to 90 and a very good herbicidal activity is given at values of 90 to 100.

The test plants used in the greenhouse experiments were of the following species:

| Bayer code | Scientific name |
|---|---|
| ALOMY | Alopercurus myosuroides |
| AMARE | Amaranthus retroflexus |
| APESV | Apera spica-venti |
| AVEFA | Avena fatua |
| ECHCG | Echinocloa crus-galli |

At an application rate of 1,000 kg/ha, applied by the pre-emergence method:
compound 14 showed very good herbicidal activity against APESV.
compounds 11, 17 showed good herbicidal activity against APESV.
compound 12 showed good herbicidal activity against AMARE.
compound 14 showed very good herbicidal activity against ECHCG.
compound 12 showed good herbicidal activity against ECHCG.

At an application rate of 1,000 kg/ha, applied by the post-emergence method:
compound 11 showed very good herbicidal activity against AMARE.
compound 17 showed very good herbicidal activity against AVEFA.
compounds 12, 13, 14, 15, 16 showed good herbicidal activity against AVEFA.
compounds 13, 14, 15 showed good herbicidal activity against ALOMY.
compounds 14, 16 showed very good herbicidal activity against ECHCG.

1. A compound of formula (I) 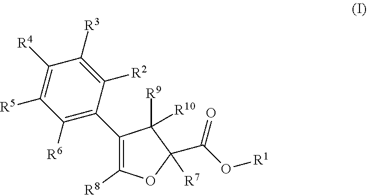
wherein the substituents have the following meanings:
R¹ hydrogen or (C₁-C₆)-alkyl, (C₃-C₆)-cycloalkyl, (C₃-C₆)-cycloalkyl-(C₁-C₃)-alkyl, (C₂-C₄)-alkenyl, (C₂-C₄)-alkynyl, each of which is substituted by m radicals selected from the group consisting of fluorine, chlorine, bromine, cyano, Z, CO₂R^{a}, CONR^{b}R^{h}, (C₁-C₂)-alkoxy, (C₁-C₂)-haloalkoxy, (C₁-C₃)-alkylthio, (C₁-C₃)-alkylsulfinyl, (C₁-C₃)-alkylsulfonyl, phenylthio, phenylsulfinyl, and phenylsulfonyl;
R² hydrogen, halogen, (C₁-C₃)-alkyl, (C₁-C₃)-haloalkyl, (C₁-C₃)-alkoxy, (C₁-C₃)-haloalkoxy;
R³ hydrogen, halogen, nitro, hydroxyl, cyano, (C₁-C₃)-alkyl, (C₁-C₃)-haloalkyl, hydroxy-(C₁-C₃)-alkyl, (C₃-C₆)-cycloalkyl, (C₃-C₆)-halocycloalkyl, hydroxy-(C₃-C₆)-cycloalkyl, (C₁-C₃)-alkoxy, (C₁-C₃)-haloalkoxy, (C₁-C₃)-alkoxycarbonyl, (C₂-C₃) alkenyl, (C₂-C₃)-haloalkenyl, (C₂-C₃) alkynyl, (C₂-C₃)-haloalkynyl, (C₁-C₃)-alkylthio, (C₁-C₃)-alkylsulfinyl, (C₁-C₃)-alkylsulfonyl;
R⁴ hydrogen, halogen, hydroxyl, cyano, (C₁-C₃)-alkyl, (C₁-C₃)-haloalkyl, (C₃-C₄)-halocycloalkyl, (C₁-C₃)-haloalkoxy, (C₂-C₃)-haloalkenyl, (C₂-C₃)-haloalkynyl;
R⁵ hydrogen, halogen, nitro, hydroxyl, cyano, (C₁-C₃)-alkyl, (C₁-C₃)-haloalkyl, hydroxy-(C₁-C₃)-alkyl, (C₃-C₆)-cycloalkyl, (C₃-C₆)-halocycloalkyl, hydroxy-(C₃-C₆)-cycloalkyl, (C₁-C₃)-alkoxy, (C₁-C₃)-haloalkoxy, (C₁-C₃)-alkoxycarbonyl, (C₂-C₃) alkenyl, (C₂-C₃)-haloalkenyl, (C₂-C₃) alkynyl, (C₂-C₃)haloalkynyl, (C₁-C₃)-alkylthio, (C₁-C₃)-alkylsulfinyl, (C₁-C₃)-alkylsulfonyl;
R⁶ hydrogen, halogen, (C₁-C₃)-alkyl, (C₁-C₃)-haloalkyl, (C₁-C₃)-alkoxy, (C₁-C₃)-haloalkoxy;
R⁷ hydrogen, halogen, cyano, or (C₁-C₆)-alkyl, (C₃-C₆)-cycloalkyl, (C₂-C₆)-alkenyl, (C₂-C₆)-alkynyl, (C₁-C₆)-alkoxy, each substituted by m radicals selected from the group consisting of fluorine, chlorine, bromine, iodine, hydroxyl, cyano, and (C₁-C₆)-alkoxy;
R⁸ hydrogen, halogen, (C₁-C₃)-alkyl, (C₁-C₃)-haloalkyl, and (C₃-C₆)-cycloalkyl;
R⁹, R¹⁰ each independently hydrogen, halogen, cyano, or (C₁-C₆)-alkyl, (C₁-C₆)-alkoxy, each substituted by m radicals selected from the group consisting of fluorine, chlorine, bromine, iodine, and cyano; or
R⁹ and R¹⁰ form, together with the carbon atom to which they are bound, a saturated, partially or fully unsaturated three to six-membered ring containing, in addition to this carbon atom, q carbon atoms and n oxygen atoms;
R^{a} (C₁-C₆)-alkyl, (C₃-C₆)-cycloalkyl, or phenyl, each of which is substituted by m radicals selected from the group consisting of fluorine, chlorine, bromine, iodine, cyano, hydroxy, and (C₁-C₃)-alkoxy;
R^{b} hydrogen, (C₁-C₃)-alkoxy or R^{a};
R^{h} hydrogen or (C₁-C₆)-alkyl, (C₁-C₂)-alkoxy, (C₃-C₆)-cycloalkyl, (C₂-C₄)-alkenyl, (C₁-C₆)-alkoxycarbonyl-(C₁-C₆)-alkyl, or (C₂-C₄)-alkynyl, each of which is substituted by m radicals selected from the group consisting of fluorine, chlorine, bromine, cyano, CO₂R^{a}, and (C₁-C₂)-alkoxy;
Z is a three-, four-, five- or six-membered saturated, partly unsaturated, fully unsaturated, or aromatic ring, which is formed from r carbon atoms, n nitrogen atoms, n sulfur atoms and n oxygen atoms, and which is substituted by m radicals selected from the group consisting of fluorine, chlorine, bromine, cyano, (C₁-C₂)-alkoxy, and (C₁-C₂)-haloalkoxy;
each m is independently 0, 1, 2, 3, 4, or 5;
each n is independently 0, 1, or 2;
q 1, 2, 3, 4, or 5;
r 1,2,3, 4, 5, or 6;
including agriculturally acceptable salts and thioesters thereof, provided the compound of formula (I) has a carboxyl group; except the compounds methyl 2,3-dihydro-4-phenyl-2-furancarboxylate and methyl 2,3-dihydro-5-methyl-4-phenyl-2-furancarboxylate.

2. The compound as claimed in claim 1, wherein the substituents have the following meaning:
R¹ hydrogen or (C₁-C₆)-alkyl, (C₃-C₆)-cycloalkyl, (C₂-C₄)-alkenyl, (C₃-C₆)-cycloalkyl-(C₁-C₃)-alkyl, (C₂-C₄)-alkynyl, each of which is substituted by m radicals selected from the group consisting of fluorine, chlorine, bromine, cyano, and Z.

3. The compound as claimed in claim 1, wherein the substituents have the following meaning:
R¹ hydrogen or (C₁-C₆)-alkyl.

4. The compound as claimed in, wherein the substituents have the following meaning:
R² hydrogen, halogen, (C₁-C₃)-alkyl;
R⁶ hydrogen, halogen, (C₁-C₃)-alkyl.

5. The compound as claimed in claim 1, wherein the substituents have the following meaning:
R³ hydrogen, halogen, hydroxyl, cyano, (C₁-C₃)-alkyl;
R⁵ hydrogen, halogen, hydroxyl, cyano, (C₁-C₃)-alkyl.

6. The compound as claimed in claim 1, wherein the substituents have the following meaning:
R⁴ hydrogen, halogen.

7. The compound as claimed in claim 1, wherein the substituents have the following meaning:
R⁷ hydrogen, (C₁-C₂)-alkyl, (C₁-C₂)-haloalkyl, (C₃-C₆)-cycloalkyl, (C₂-C₆)-alkenyl, (C₂-C₆)-alkynyl, (C₁-C₆)-alkoxy;
R⁸ hydrogen or halogen.

8. The compound as claimed in claim 1, wherein the substituents have the following meaning:
R⁹ hydrogen, halogen, (C₁-C₃)-alkyl, (C₁-C₃)-haloalkyl;
R¹⁰ hydrogen, halogen, (C₁-C₃)-alkyl, (C₁-C₃)-haloalkyl.

9. The compound as claimed in claim 1,
wherein the substituents have the following meaning:
R¹ hydrogen or (C₁-C₆)-alkyl;
R² hydrogen;
R³ halogen, cyano, (C₁-C₃)-alkyl, (C₁-C₃)-haloalkyl, (C₁-C₃)-haloalkoxy;
R⁴ hydrogen or halogen;
R⁵ halogen, cyano, (C₁-C₃)-alkyl, (C₁-C₃)-haloalkyl, (C₁-C₃)-haloalkoxy;
R⁶ hydrogen;
R⁷ (C₁-C₆)-alkyl, (C₃-C₆)-cycloalkyl, (C₂-C₆)-alkenyl, (C₂-C₆)-alkynyl, (C₁-C₆)-alkoxy, each substituted by m radicals selected from the group consisting of fluorine, chlorine, bromine, iodine, hydroxyl, cyano, and (C₁-C₆)-alkoxy;
R⁸ hydrogen, halogen, (C₁-C₃)-alkyl, (C₁-C₃)-haloalkyl;
R⁹, R¹⁰ each independently hydrogen, halogen, cyano, or (C₁-C₆)-alkyl, (C₁-C₆)-alkoxy, each substituted by m radicals selected from the group consisting of fluorine, chlorine, bromine, iodine, and cyano; or
R⁹ and R¹⁰ form, together with the carbon atom to which they are bound, a saturated, partially or fully unsaturated three to six-membered ring containing, in addition to this carbon atom, q carbon atoms, and n oxygen atoms;
each m is independently 0, 1, 2, 3, 4, or 5;
each n is independently 0, 1, or 2;
q 1, 2, 3, 4, or 5.

10. A composition comprising at least one compound as claimed in claim 1, and at least one auxiliary, which is customary for formulating crop protection compounds.

11. The composition as claimed in claim 10, comprising a further herbicide.

12. (canceled)

13. A method for controlling unwanted vegetation comprising applying an herbicidally effective amount of at least one compound as claimed in claim 1 to act on plants, their seed, and/or their habitat.
